(19) United States
(12) Patent Application Publication
(10) Pub. No.: US 2025/0368635 A1
(43) Pub. Date: Dec. 4, 2025

(54) HETEROARYL DERIVATIVE COMPOUND, AND USES THEREOF

(71) Applicant: VORONOI, INC., Incheon (KR)

(72) Inventors: Younho LEE, Incheon (KR); Seonah HWANG, Incheon (KR); Daekwon KIM, Incheon (KR)

(73) Assignee: VORONOI, INC., Incheon (KR)

(21) Appl. No.: 19/123,737

(22) PCT Filed: Nov. 14, 2023
(86) PCT No.: PCT/KR2023/018285

§ 371 Date: Apr. 23, 2025

(30) Foreign Application Priority Data
Nov. 15, 2022 (KR) 10-2022-0152950

(51) Int. Cl. C07D 413/14; A61K 31/506; A61K 31/5377; C07D 471/10; C07D 487/04; C07D 491/048; C07D 491/107; C07D 498/04; C07D 498/08

(52) U.S. Cl. CPC C07D 413/14; A61K 31/506; A61K 31/5377; C07D 471/10; C07D 487/04; C07D 491/048; C07D 491/107; C07D 498/04; C07D 498/08

(57) ABSTRACT

The present invention relates to heteroaryl derivatives and uses thereof. The heteroaryl derivative of the present invention exhibits excellent inhibitory activity against EGFR and/or HER2, which may be usefully employed as a therapeutic agent for EGFR- and/or HER2-related diseases.

### TECHNICAL FIELD

The present invention relates to a heteroaryl derivative compound and medicinal uses thereof. Specifically, the present invention relates to a heteroaryl derivative compound having EGFR and/or HER2 inhibitory activity.

### BACKGROUND ART

Protein kinases act as molecular switches to participate in signal transduction pathways, and the transition between active and inactive states of target proteins by kinases in cells should be smoothly controlled. If the transition between the active and inactive states is abnormally controlled, intracellular signal transduction is excessively activated or deactivated to induce uncontrollable cell division and proliferation. In particular, abnormal activation by mutation, amplification and/or overexpression of protein kinase genes causes the development and progression of various tumors or plays a crucial role in the development of various diseases such as inflammatory diseases, degenerative brain diseases, and autoimmune diseases.

EGFR (epidermal growth factor receptor), which is a receptor tyrosine kinase of the ErbB family, is abnormally active in many epithelial cell tumors, including non-small cell lung carcinoma (NSCLC), breast cancer, glioma, squamous cell carcinoma of the head and neck, colorectal cancer, rectal adenocarcinoma, head and neck cancer, stomach cancer and prostate cancer, and the activation of the EGFR-tyrosine kinase causes continuous cell proliferation, invasion of surrounding tissues, distant metastasis, and angiogenesis, and is known to increase cell survival.

In addition, it has been known that the EGFR mutation, EGFR Del19 or EGFR L858R, is a major cause of non-small cell lung cancer and head and neck cancer, and Iressa and Tarceva as therapeutic agents thereof have been developed and are currently used in clinical practice. However, in patients injected with these drugs, acquired resistance, which causes EGFR secondary mutations based on the structure of the drug, was observed, and it was also found that this resistance is a major cause of actual drug resistance. When first-generation EGFR inhibitors are administered for about 10 months on average, acquired resistance, called the T790M mutation located in the gatekeeper of EGFR kinase, occurs, and thus the first-generation EGFR inhibitors show no drug efficacy. In other words, EGFR Del19/T790M or EGFR L858R/T790M double mutation occurs, preventing the existing therapeutic agents from exhibiting drug efficacy. In this regard, Osimertinib, a third-generation EGFR-TKI target drug that shows high reactivity against drug resistance due to the EGFR T790M mutation, has been developed, but this drug has also been reported to have drug resistance (Niederst M J. et al., Clin Cancer Res, 2015, 17 (21): 3924-3933). The EGFR C797S mutation has been suggested as one of the main mechanisms that cause drug resistance to Osimertinib, and about 40% of clinical trial patients have been reported to have the EGFR C797S mutation (Thress K S. et al., Nature Medicine, 2015, 21:560-562). Therefore, EGFR Del19/C797S (EGFR DC) or EGFR L858R/C797S (EGFR LC) may be the major target.

In addition, L861Q, G719A, S768I, L718Q, G724S, etc., that express drug-resistant mutations and rare or uncommon EGFR mutations may also be potential targets.

On the other hand, HER2 (Human epidermal growth factor receptor 2; also known as ErbB2), which is a receptor tyrosine kinase of the ErbB family, forms homodimers or heterodimers with other EGFR receptors such as HER1 (EGFR, ErbB1), HER3 (ErbB3), or HER4 (ErbB4) and is activated by autophosphorylation at intracellular tyrosine residues to play an important role in cell proliferation, differentiation and survival in normal cells and cancer cells (Di Fiore P P, et al., Science. 1987; 237 (481): 178-182). HER2 is known to be overexpressed in various carcinomas such as breast cancer, gastric cancer and ovarian cancer (Hardwick R H, et al., Eur. J Surg Oncol. 1997, 23 (1): 30-35; Korkaya H. et al., Oncogene. 2008, 27 (47): 6120-6130).

As described above, there is an increasing unmet need for novel compounds capable of being usefully utilized in the treatment of EGFR and/or HER2-related diseases by modulating EGFR activity (especially C797S mutations such as EGFR Del19/C797S, and EGFR L858R/C797S, EGFR rare mutation, or drug resistance mutation, etc.) and/or HER2.

### DISCLOSURE OF INVENTION

### Technical Problem

An object of the present invention is to provide a heteroaryl derivative having a novel structure, a stereoisomer thereof, or a pharmaceutically acceptable salt thereof.

Another object of the present invention is to provide a method for preparing the heteroaryl derivative compound.

Still another object of the present invention is to provide a pharmaceutical use of the heteroaryl derivative compound, and specifically, to a pharmaceutical composition for the treatment or prevention of EGFR- and/or HER2-related diseases comprising the heteroaryl derivative compound as an active ingredient, use of the compound for the treatment or prevention of EGFR- and/or HER2-related diseases, or a method for treating or preventing EGFR- and/or HER2-related diseases comprising administering the compound.

### Solution to Problem

In order to achieve the above object, the present inventors have made research efforts, and as a result, completed the present invention by confirming that a heteroaryl derivative compound represented by the following Chemical Formula 1, 2, 2a, 2b, or 3 inhibited the proliferation of EGFR- and/or HER2-activated cells.

### Heteroaryl Derivative Compound

The present invention provides a compound represented by the following Chemical Formula 1, a stereoisomer thereof, or a pharmaceutically acceptable salt thereof:

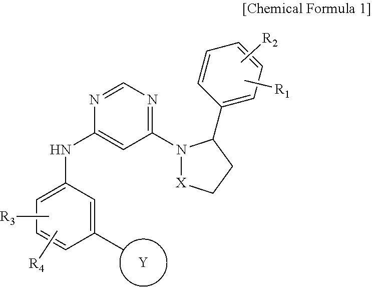

in Chemical Formula 1 above,
X is CH₂ or O;
R₁ and R₂ are each independently —H, —C₁₋₆alkyl, —C₁₋₆haloalkyl, or -halo;
R₃ is —H, —C₁₋₆alkyl, —C₁₋₆haloalkyl, —O—C₁₋₆alkyl, or —O—C₁₋₃alkyl-OH {wherein R₃ may be linked to a carbon in the phenyl ring to form a fused ring or linked to R₄ to form a fused ring};
R₄ is —H, —NH(C₁₋₆alkyl), —N(C₁₋₆alkyl)(C₁₋₆alkyl), -(4-6 membered heterocycloalkyl), or -(7-11 membered heterobicycloalkyl) {wherein the —NH(C₁₋₆alkyl) or —N(C₁₋₆ alkyl)(C₁₋₆alkyl) may be linked to the ring Y to form a fused ring; at least one H of the -(4-6 membered heterocycloalkyl) may be substituted with —C₁₋₆alkyl, —C₁₋₆haloalkyl, —(CH₂)n-O—C₁₋₆alkyl, —(CH₂)n-NRₐR_{b}, —(CH₂)n-S(═O)₂—C₁₋₃alkyl, -halo, —(CH₂)n-(4-6 membered heterocycloalkyl), or —(CH₂)n-(7-11 membered heterobicycloalkyl) [here, at least one H of the —(CH₂)n-(4-6 membered heterocycloalkyl) or —(CH₂)n-(7-11 membered heterobicycloalkyl) ring may be substituted with —C₁₋₆alkyl, —C₁₋₆haloalkyl, —(CH₂)n-O—C₁₋₆alkyl, —(CH₂)n-C(═O)—C₁₋₃alkyl, —(CH₂)n-S(═O)₂—C₁₋₃alkyl, —(CH₂)nNRₐR_{b}, or -halo]; at least one H of the -(7-11 membered heterobicycloalkyl) may be substituted with —C₁₋₆alkyl, —C₁₋₆haloalkyl, —(CH₂)n-C(═O)—C₁₋₃alkyl, —(CH₂)n-C(═O)-(3-6 membered cycloalkyl), —(CH₂)n-S(═O)₂—C₁₋₃alkyl, —(CH₂)n-NRₐR_{b}, —O—C₁₋₆alkyl, -halo, or —(CH₂)n-(4-6 membered heterocycloalkyl) [here, at least one H of the —(CH₂)n-(4-6 membered heterocycloalkyl) or —(CH₂)n-C(═O)-(3-6 membered cycloalkyl) ring may be substituted with —C₁₋₆alkyl, —C₁₋₆haloalkyl, or -halo]};
n is 0, 1, 2, 3, or 4;
Rₐ and R_{b} are each independently —H, —C₁₋₆alkyl, —C₁₋₆haloalkyl, or -(4-6 membered heterocycloalkyl);
ring Y is a phenyl or 5-10 membered heteroaryl {wherein at least one H of the phenyl or 5-10 membered heteroaryl ring may be substituted with —C₁₋₆alkyl, —C₁₋₆ hydroxyalkyl, —C₁₋₆haloalkyl, -(3-6 membered cycloalkyl), or -halo}.

According to an embodiment of the present invention, the compound represented by Chemical Formula 1 may be in the following range:
X is CH₂ or O;
R₁ and R₂ are each independently —H or -halo;
R₃ is —H, —C₁₋₃haloalkyl, —O—C₁₋₃alkyl, or —O—C₁₋₃alkyl-OH {wherein R₃ may be linked to a carbon in the phenyl ring to form a fused ring, or linked to R₄ to form a fused ring};
R₄ is —H, —N(C₁₋₃alkyl)(C₁₋₃alkyl), -(4-6 membered heterocycloalkyl), or -(7-11 membered heterobicycloalkyl) {wherein the —N(C₁₋₃alkyl)(C₁₋₃alkyl) may be linked to the ring Y to form a fused ring; at least one H of the -(4-6 membered heterocycloalkyl) may be substituted with —C₁₋₆alkyl, —C₁₋₆haloalkyl, —(CH₂)n-O—C₁₋₃alkyl, —(CH₂)n-NRₐR_{b}, (CH₂)n-S(═O)₂—C₁₋₃alkyl, -halo, —(CH₂)n-(4-6 membered heterocycloalkyl), or —(CH₂) n-(7-11 membered heterobicycloalkyl) [here, at least one H of the —(CH₂)n-(4-6 membered heterocycloalkyl) or —(CH₂)n-(7-11 membered heterobicycloalkyl) ring may be substituted with —C₁₋₃alkyl, —C₁₋₆haloalkyl, —(CH₂)n-O—C₁₋₃alkyl, —(CH₂)n-S(═O)₂—C₁₋₃ alkyl, —(CH₂)nNRₐR_{b}, or -halo]; at least one H of the -(7-11 membered heterobicycloalkyl) may be substituted with —C₁₋₃alkyl, —C₁₋₆haloalkyl, —(CH₂)n-C(═O)—C₁₋₃alkyl, (CH₂)n-C(═O)-(3-6 membered cycloalkyl), —(CH₂)n-S(═O)₂—C₁₋₃alkyl, —(CH₂)n-NRₐR_{b}, —O—C₁₋₃alkyl, -halo, or —(CH₂)n-(4-6 membered heterocycloalkyl) [here, at least one H of the —(CH₂)n-(4-6 membered heterocycloalkyl) or —(CH₂)n-C(═O)-(3-6 membered cycloalkyl) ring may be substituted with -halo]};
n is 0, 1, 2, or 3;
Rₐ and R_{b} are each independently —H, —C₁₋₃alkyl, —C₁₋₆haloalkyl, or -(4-6 membered heterocycloalkyl);
ring Y is a phenyl, 5-6 membered heteroaryl, or 9-10 membered heteroaryl {wherein at least one H of the phenyl, 5-6 membered heteroaryl, or 9-10 membered heteroaryl ring may be substituted with —C₁₋₃alkyl, —C₁₋₃hydroxyalkyl, -(3-6 membered cycloalkyl), or -halo}.

According to an embodiment of the present invention, the compound represented by Chemical Formula 1 may be in the following range:

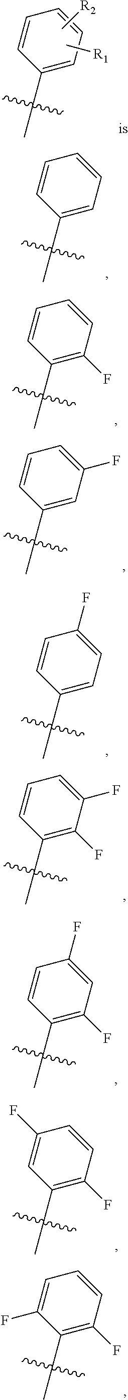

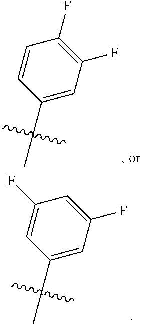 [SMILES: *c1ccc(F)c(F)c1 ; *c1cc(F)cc(F)c1]

According to an embodiment of the present invention, the compound represented by Chemical Formula 1 may be in the following range:
ring Y is

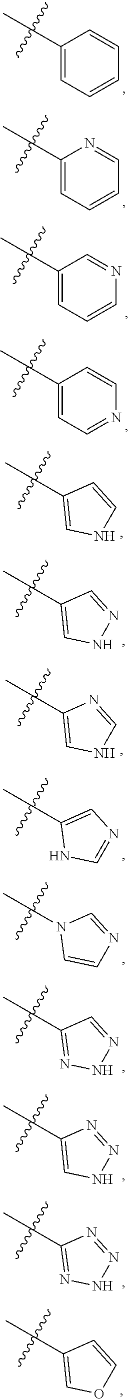

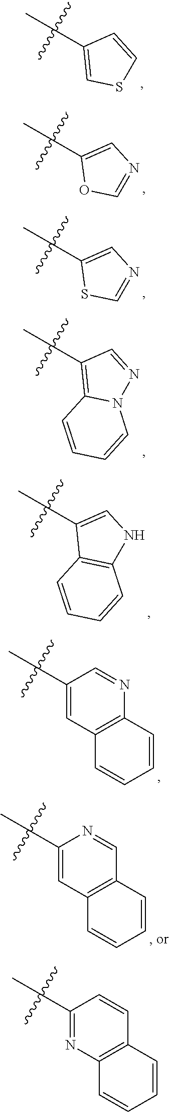

{wherein at least one H of the ring Y may be substituted with —C₁₋₃alkyl, —C₁₋₃hydroxyalkyl, —C₁₋₃ haloalkyl, -(3-6 membered cycloalkyl), or -halo}.

Further, the present invention provides a compound represented by the following Chemical Formula 2, a stereoisomer thereof, or a pharmaceutically acceptable salt thereof:

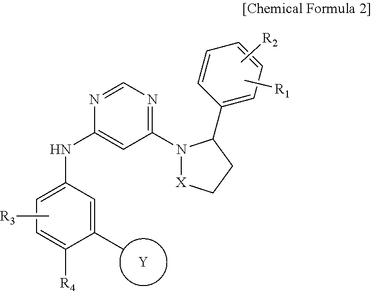

in Chemical Formula 2 above,
X is CH₂ or O;
R₁ and R₂ are each independently —H or -halo;
R₃ is —H, —C₁₋₃haloalkyl, —O—C₁₋₃alkyl, or —O—C₁₋₃alkyl-OH, {wherein R₃ may be linked to a carbon in the phenyl ring to form a fused ring, or linked to R₄ to form a fused ring};
R₄ is —H, —N(C₁₋₃alkyl)(C₁₋₃alkyl), -(4-6 membered heterocycloalkyl), or -(7-11 membered heterobicycloalkyl) {wherein the —N(C₁₋₃alkyl)(C₁₋₃alkyl) may be linked to the ring Y to form a fused ring; at least one H of the -(4-6 membered heterocycloalkyl) may be substituted with —C₁₋₆alkyl, —C₁₋₆haloalkyl, —(CH₂)n-O—C₁₋₃alkyl, —(CH₂)n-NRₐR_{b},
(CH₂)n-S(═O)₂—C₁₋₃alkyl, -halo, —(CH₂)n-(4-6 membered heterocycloalkyl), or —(CH₂) n-(7-11 membered heterobicycloalkyl) [here, at least one H of the —(CH₂)n-(4-6 membered heterocycloalkyl) or —(CH₂)n-(7-11 membered heterobicycloalkyl) ring may be substituted with —C₁₋₃alkyl, —C₁₋₆haloalkyl, —(CH₂)n-O—C₁₋₃alkyl, —(CH₂)n-S(═O)₂—C₁₋₃ alkyl, —(CH₂)nNRₐR_{b}, or -halo]; at least one H of the -(7-11 membered heterobicycloalkyl) may be substituted with —C₁₋₃alkyl, —C₁₋₆haloalkyl, —(CH₂)n-C(═O)—C₁₋₃alkyl, —(CH₂)n-C(═O)-(3-6 membered cycloalkyl), —(CH₂)n-S(═O)₂—C₁₋₃alkyl, —(CH₂)n-NRₐR_{b}, —O—C₁₋₃alkyl, -halo, or —(CH₂)n-(4-6 membered heterocycloalkyl) [here, at least one H of the —(CH₂)n-(4-6 membered heterocycloalkyl), or —(CH₂)n-C(═O)-(3-6 membered cycloalkyl) ring may be substituted with -halo]};
n is 0, 1, 2, or 3;
Rₐ and R_{b} are each independently —H, —C₁₋₃alkyl, —C₁₋₆haloalkyl, or -(4-6 membered heterocycloalkyl);
ring Y is a phenyl, 5-6 membered heteroaryl, or 9-10 membered heteroaryl {wherein at least one H of the phenyl, 5-6 membered heteroaryl, or 9-10 membered heteroaryl ring may be substituted with —C₁₋₃alkyl, —C₁₋₃hydroxyalkyl, -(3-6 membered cycloalkyl), or -halo}.

Further, the present invention provides a compound represented by the following Chemical Formula 2a, a stereoisomer thereof, or a pharmaceutically acceptable salt thereof:

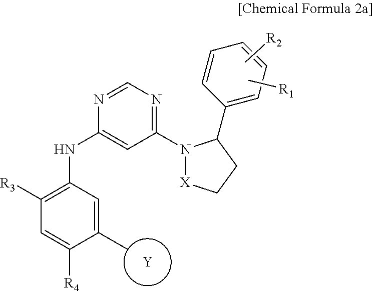

in Chemical Formula 2a above,
X, R₁, R₂, R₃, R₄, and ring Y are the same as defined in Chemical Formula 2 above.

Further, the present invention provides a compound represented by the following Chemical Formula 2b, a stereoisomer thereof, or a pharmaceutically acceptable salt thereof:

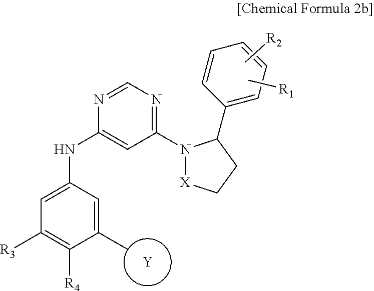

in Chemical Formula 2b above,
X, R₁, R₂, R₃, R₄, and ring Y are the same as defined in Chemical Formula 2 above.

In addition, the present invention provides a compound represented by the following Chemical Formula 3, a stereoisomer thereof, or a pharmaceutically acceptable salt thereof:

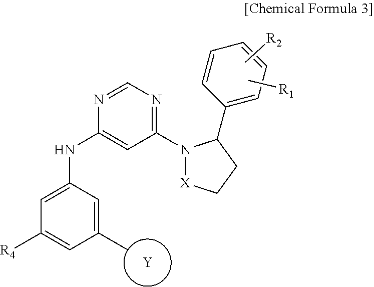

in Chemical Formula 3 above,
X is CH₂ or O;
R₁ and R₂ are each independently —H or -halo;
R₄ is -(4-6 membered heterocycloalkyl) or -(7-11 membered heterobicycloalkyl) {wherein at least one H of the -(4-6 membered heterocycloalkyl) or -(7-11 membered heterobicycloalkyl) ring may be substituted with —C₁₋₃alkyl, —C₁₋₃haloalkyl, —(CH₂)n-S(═O)₂—C₁₋₃alkyl, or -halo};
n is 0, 1, or 2;
ring Y is a 5 membered heteroaryl {wherein at least one H of the 5 membered heteroaryl ring may be substituted with —C₁₋₃alkyl, or -halo}.

According to an embodiment of the present invention, the compound represented by Chemical Formula 1, 2, 2a, 2b, or 3 may be selected from the group consisting of compounds listed in Table 1 described below.

In the present invention, unless otherwise specified, the term “alkyl” may refer to a straight or branched chain acyclic, cyclic, or saturated hydrocarbon to which they are bonded. For example, “C₁₋₆alkyl” may indicate an alkyl containing 1 to 6 carbon atoms. As an example, acyclic alkyl may include, but is not limited to, methyl, ethyl, n-propyl, n-butyl, isopropyl, sec-butyl, isobutyl, tert-butyl, or the like. Cyclic alkyl may be used interchangeably with “cycloalkyl” as used herein, and as an example, may include, but is not limited to, cyclopropyl, cyclobutyl, cyclopentyl, cyclohexyl, cycloheptyl, cyclooctyl, or the like.

In the present invention, “alkoxy” may indicate-(O-alkyl) as an alkyl ether group, wherein alkyl is the same as defined above. For example, “C₁₋₆alkoxy” may mean alkoxy containing C₁₋₆alkyl, that is, —(O—C₁₋₆alkyl), and as an example, may include, but is not limited to, methoxy, ethoxy, n-propoxy, isopropoxy, n-butoxy, isobutoxy, secbutoxy, tert-butoxy, or the like.

In the present invention, “halo” may be F, Cl, Br, or I.

In the present invention, “haloalkyl” may mean a straight or branched chain alkyl (hydrocarbon) having one or more halo-substituted carbon atoms as defined herein. Examples of the haloalkyl may include, but are not limited to, methyl, ethyl, propyl, isopropyl, isobutyl or n-butyl independently substituted with one or more halogens, such as F, Cl, Br, or I.

In the present specification, “hydroxyalkyl” may indicate a straight or branched chain alkyl (hydrocarbon) having a carbon atom substituted with -hydroxy (—OH). Examples of the hydroxyalkyl may include, but are not limited to, methyl, ethyl, propyl, isopropyl, isobutyl or n-butyl independently substituted with one or more, for example —OH.

In the present specification, “aminoalkyl” may mean a straight or branched chain alkyl (hydrocarbon) having a carbon atom substituted with amino (NR′R″). Here, R′ and R″ may be each independently selected from the group consisting of hydrogen and C₁₋₆alkyl, and the selected R′ and R″ may be each independently substituted or unsubstituted.

In the present specification, “cyanoalkyl” may refer to a straight or branched chain alkyl (hydrocarbon) having carbon atoms substituted with cyano (CN).

In the present invention, “cycloalkyl” may refer to a hydrocarbon ring that does not contain a hetero atom (N, O, P, P(═O), S, or the like) in the ring, and may be saturated or partially unsaturated. Here, when unsaturated, the cycloalkyl may be referred to as a cycloalkenyl. Unless otherwise stated, the cycloalkyl may be a single ring or multiple rings such as a spiro ring, a bridged ring or a fused ring.

In the present invention, “heterocycloalkyl” may mean a ring containing at least one selected from N, O, P, P(═O), and S in the ring and may be saturated or partially unsaturated. Here, when unsaturated, it may be referred to as a heterocycloalkene. Unless otherwise stated, heterocycloalkyl may be a single ring. In addition, “3- to 12-membered heterocycloalkyl” may indicate a heterocycloalkyl containing 3 to 12 atoms forming a ring. As an example, the heterocycloalkyl may include, but is not limited to, pyrrolidine, piperidine, imidazolidine, pyrazolidine, butyrolactam, valerolactam, imidazolidinone, hydantoin, dioxolane, phthalimide, piperidine, pyrimidin-2,4 (1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, and the like.

In the present invention, “heterobicycloalkyl” may mean multiple rings such as a spiro ring, a bridged ring or a fused ring containing at least one selected from N, O, P, P(═O), and S in the rings, and may be saturated or partially unsaturated. Herein, when unsaturated, the heterobicycloalkyl may be referred to as a heterobicycloalkene. Examples of the heterobicycloalkyl may include, but are not limited to, quinuclidine, trophane, 2-azaspiro[3.3]heptane, (1r,5s)-3-azabicyclo[3.2.1]octane, (1s,4s)-2-azabicyclo[2.2.2]octane, or (1r,4r)-2-oxa-5-azabicyclo[2.2.2]octane, and the like.

In the present invention, “arene” may mean an aromatic hydrocarbon ring. The arene may be a single ring or multiple rings. The number of ring carbon atoms in the arene may be 5 or more and 30 or less, 5 or more and 20 or less, or 5 or more and 15 or less. Examples of arene may include, but are not limited to, benzene, naphthalene, fluorene, anthracene, phenanthrene, bibenzene, terbenzene, quaterbenzene, quinquebenzene, sexibenzene, triphenylene, pyrene, benzofluoranthene, chrysene, and the like. In the present specification, a moiety obtained by removing one hydrogen atom from the above “arene” is referred to as “aryl”.

In the present invention, “heteroarene” may be a ring containing at least one or more of O, N, P, Si, and S as a heterogeneous element. The number of ring-forming carbons in the heteroarene may be 2 or more and 30 or less, or 2 or more and 20 or less. The heteroarene may be a monocyclic heteroarene or a polycyclic heteroarene. The polycyclic heteroarene may have, for example, a bicyclic or tricyclic structure.

Examples of the heteroarene may include thiophene, purine, pyrrole, pyrazole, imidazole, thiazole, oxazole, isothiazole, oxadiazole, triazole, pyridine, pyridin-2-one, pyridin-3-one, pyridin-4-one, bipyridyl, triazine, acridyl, pyridazine, pyrazine, quinoline, quinazoline, quinoxaline, phenoxazine, phthalazine, pyrimidine, pyridopyrimidine, pyridopyrazine, pyrazinopyrazine, isoquinoline, indole, carbazole, imidazopyridazine, imidazopyridine, imidazopyrimidine, pyrazolopyrimidine, imidazopyrazine, or pyrazolopyridine, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, isoxazole, oxadiazole, thiadiazole, benzothiazole, tetrazole, phenothiazine, dibenzosilole, dibenzofuran, and the like, but are not limited thereto. In an embodiment of the present invention, heteroarene may also include bicyclic heterocyclo-arene containing heteroarene fused to an arene ring or a cycloalkyl ring fused to heterocycloalkyl rings. In the present specification, the residue obtained by removing one hydrogen atom from the “heteroarene” is referred to as “heteroaryl”.

In the present invention, “hydroarene” or “hydroaryl” may be a ring saturated one or more double bonds in an aromatic hydrocarbon ring.

In the present invention, “heterohydroarene” or “heterohydroaryl” may be a ring saturated one or more double bonds in a “heteroarene” or “heteroaryl” ring.

In the present invention, “ring” may be a single ring or multiple rings, and the multiple rings may be in the form of a spiro ring, a bridged ring, or a fused ring.

In the present invention, the term “stereoisomer” means a compound of the present invention, having the same chemical formula or molecular formula but different in spatial arrangement. In the present specification, the stereoisomer includes an optical isomer, an enantiomer, a diastereomer, cis/trans isomer, rotamer, and atropisomer. Each of these isomer, racemate and mixture thereof are also included within the scope of the present invention. For example, Chemical Formula 1, 2, 2a, 2b, or 3 of the present invention may include stereoisomers of Chemical Formula 1, 2, 2a, 2b, or 3 because the stereochemistry is not specified. Unless otherwise specified, a solid bond ( [symbol] ) connected to an asymmetric carbon atom may include a wedged solid bond ( [symbol] ) or wedge dashed bond ( [symbol] ) representing the absolute arrangement of stereocenters.

The compound represented by Chemical Formula 1, 2, 2a, 2b, or 3 of the present invention may be present in the form of a “pharmaceutically acceptable salt”. Thus, pharmaceutically acceptable salts of the compound represented by Chemical Formula 1, 2, 2a, 2b, or 3 above are included in the scope of the compound of the present invention. The term “pharmaceutically acceptable salt” as used herein refers to a concentration having a relatively non-toxic and harmless effective effect on patients, which includes any organic acid or inorganic acid addition salt of the compound represented by Chemical Formula 1, 2, 2a, 2b, or 3 in which side effects caused by these salts do not reduce the beneficial efficacy of the compound.

In particular, the pharmaceutically acceptable salt may be an acid addition salt formed from a free acid. Here, the acid addition salts may be obtained from inorganic acids such as hydrochloric acid, nitric acid, phosphoric acid, sulfuric acid, hydrobromic acid, hydroiodic acid, nitrous acid, phosphorous acid, and the like, nontoxic organic acids such as aliphatic mono- and dicarboxylates, phenyl-substituted alkanoates, hydroxy alkanoates and alkanedioates, aromatic acids, aliphatic and aromatic sulfonic acids, and organic acids such as trifluoroacetic acid, acetate, benzoic acid, citric acid, lactic acid, maleic acid, gluconic acid, methanesulfonic acid, 4-toluenesulfonic acid, tartaric acid, fumaric acid, and the like.

Examples of the pharmaceutically acceptable salt may include sulfate, sulfite, nitrate, phosphate, pyrophosphate, chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, benzoate, phthalate, benzenesulfonate, toluenesulfonate, phenylacetate, phenylpropionate, phenylbutyrate, citrate, lactate, glycolate, malate, tartrate, mandelate, and the like.

The acid addition salt may be prepared by a conventional method, for example, may be prepared by dissolving the compound represented by Chemical Formula 1, 2, 2a, 2b, or 3 in an organic solvent such as methanol, ethanol, acetone, methylene chloride, acetonitrile, or the like, and adding an organic acid or an inorganic acid to form a precipitate, followed by filtering and drying the resulting precipitate, or may be prepared by distilling a solvent and excess acid under reduced pressure, followed by drying and crystallizing the product in an organic solvent.

Further, the pharmaceutically acceptable salt may be a salt obtained using a base or a metal salt. As an example of the metal salt, an alkali metal salt or an alkaline earth metal salt may be obtained by dissolving the compound in an excess alkali metal hydroxide or alkaline earth metal hydroxide solution, and filtering an insoluble compound salt, followed by evaporating and drying the filtrate. Sodium, potassium or calcium salts may be pharmaceutically suitable as alkali metal salts. In addition, the corresponding silver salt may be obtained by reacting an alkali metal or alkaline earth metal with a suitable silver salt (for example, silver nitrate) or may be prepared by salt production methods known in the art.

### Use of Heteroaryl Derivative Compound

The present invention provides use of a compound represented by the following Chemical Formula 1, 2, 2a, 2b, or 3, a stereoisomer thereof, or a pharmaceutically acceptable salt thereof.

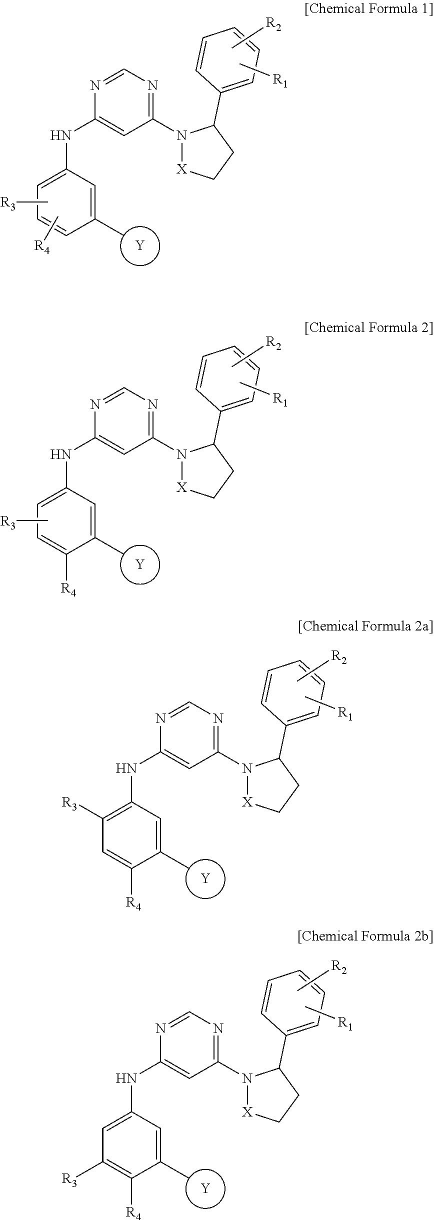

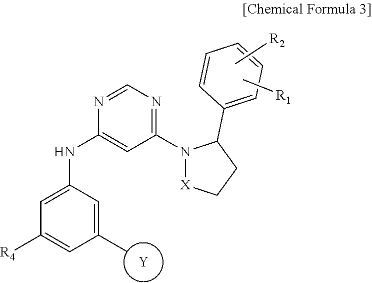

The compound represented by Chemical Formula 1, 2, 2a, 2b, or 3 of the present invention, the stereoisomer thereof, or the pharmaceutically acceptable salt thereof exhibits inhibitory activity against various kinases.

According to an embodiment of the present invention, the heteroaryl derivative represented by Chemical Formula 1, 2, 2a, 2b, or 3 exhibit excellent inhibitory activity against EGFR and/or HER2 kinases, thereby being usefully employed for treatment or prevention of EGFR- and/or HER2-related diseases, in particular, cancer. Specifically, the compound of Chemical Formula 1, 2, 2a, 2b, or 3 may inhibit EGFR and/or HER2 wild-type or mutant kinase, which is supported by Experimental Examples to be described below. The EGFR mutation may be a C797S mutation such as EGFR Del19/C797S (EGFR DC) or EGFR L858R/C797S (EGFR LC), but is not limited thereto. Further, the EGFR mutation may be EGFR L861Q, EGFR G719A, EGFR S768I, EGFR L718Q, or EGFR G724S, but is not limited thereto. In addition, the EGFR mutation may be EGFR d746-750, EGFR d746-750/C797A, EGFR d746-750/C797S, EGFR d746-750/T790M/C797S, EGFR D761Y, EGFR G719C, EGFR G719D, EGFR G719S, EGFR L747S, EGFR L792F, EGFR L858R, or EGFR L792F/L858R, but is not limited thereto.

In the present invention, the cancer includes any cancer capable of exhibiting therapeutic or prophylactic efficacy due to inhibition of HER2 and/or EGFR kinase activity, and may be a solid cancer or a hematologic cancer. The type of cancer is not limited thereto, but may be selected one or more from the group consisting of, for example, pseudomyxoma, intrahepatic cholangiocarcinoma, hepatoblastoma, liver cancer, thyroid cancer, colon cancer, testicular cancer, myelodysplastic syndrome, glioblastoma, oral cancer, lip cancer, mycosis fungoides, acute myeloid leukemia, acute lymphoblastic leukemia, basal cell carcinoma, ovarian epithelial cancer, ovarian germ cell tumor, male breast cancer, brain cancer, pituitary adenoma, multiple myeloma, gallbladder cancer, biliary tract cancer, colorectal cancer, chronic myelogenous leukemia, chronic lymphocytic leukemia, retinoblastoma, choroidal melanoma, ampulla of Vater cancer, bladder cancer, peritoneal cancer, parathyroid cancer, adrenal cancer, sinonasal cancer, non-small cell lung cancer, tongue cancer, astrocytoma, small cell lung cancer, childhood brain cancer, childhood lymphoma, childhood leukemia, small bowel cancer, meningioma, esophageal cancer, glioma, renal pelvis cancer, renal cancer, renal cell carcinoma, heart cancer, duodenal cancer, malignant soft tissue tumor, malignant bone tumor, malignant lymphoma, malignant mesothelioma, malignant melanoma, eye cancer, vulvar cancer, ureteral cancer, urethral cancer, cancer of unknown primary site, gastric lymphoma, gastric cancer, gastric carcinoid tumor, Gastrointestinal stromal tumor, Wilms' tumor, breast cancer, sarcoma, penile cancer, pharyngeal cancer, gestational choriocarcinoma, cervical cancer, endometrial cancer, uterine sarcoma, prostate cancer, metastatic bone cancer, metastatic brain cancer, mediastinal cancer, rectal cancer, rectal carcinoid tumor, vaginal cancer, spinal cord cancer, vestibular schwannoma, pancreatic cancer, salivary gland cancer, Kaposi's sarcoma, Paget's disease, tonsil cancer, squamous cell carcinoma, lung adenocarcinoma, lung cancer, squamous cell carcinoma of lung, skin cancer, anal cancer, rhabdomyosarcoma, laryngeal cancer, pleura cancer, hematological cancer, and thymic carcinoma. The cancer includes not only primary cancer but also metastatic cancer.

According to an embodiment of the present invention, the present invention provides a pharmaceutical composition for treatment or prevention of EGFR- and/or HER2-related diseases containing the compound represented by Chemical Formula 1, 2, 2a, 2b, or 3, the stereoisomer thereof, or the pharmaceutically acceptable salt thereof as an active ingredient. Specifically, the EGFR- and/or HER2-related disease may be cancer. The types of cancer are the same as described above.

The pharmaceutical composition of the present invention may further include one or more active ingredients exhibiting the same or similar drug efficacy in addition to the compound represented by Chemical Formula 1, 2, 2a, 2b, or 3 above, the stereoisomer thereof, or the pharmaceutically acceptable salt thereof.

The pharmaceutical composition of the present invention can be used for clinical administration, and may be prepared to be administrated in various oral and parenteral dosage forms.

In addition, according to an embodiment of the present invention, the present invention provides use of the compound represented by Chemical Formula 1, 2, 2a, 2b, or 3 above, the stereoisomer thereof, or the pharmaceutically acceptable salt thereof for the manufacture of a medicament for the treatment or prevention of EGFR- and/or HER2-related diseases. Specifically, the EGFR- and/or HER2-related diseases may be a cancer. The types of cancer are the same as described above.

In addition, according to an embodiment of the present invention, the present invention provides use of the compound represented by Chemical Formula 1, 2, 2a, 2b, or 3 above, the stereoisomer thereof, or the pharmaceutically acceptable salt thereof for use in preparation of a medicament to treat or prevent diseases of cancer. The types of cancer are the same as described above.

In addition, according to an embodiment of the present invention, the present invention provides a method for treating or preventing EGFR- and/or HER2-related diseases, comprising: administering a therapeutically effective amount of the compound represented by Chemical Formula 1, 2, 2a, 2b, or 3 above, the stereoisomer thereof, or the pharmaceutically acceptable salt thereof to a subject in need thereof. The subject may be a mammal including a human. Specifically, the EGFR- and/or HER2-related diseases may be cancer. The types of cancer are the same as described above.

In addition, according to an embodiment of the present invention, the present invention provides a method for treating or preventing cancer, comprising: administering a therapeutically effective amount of the compound represented by Chemical Formula 1, 2, 2a, 2b, or 3, the stereoisomer thereof, or the pharmaceutically acceptable salt thereof to a subject in need thereof. The types of cancer are the same as described above.

In addition, according to an embodiment of the present invention, the present invention provides a method for inhibiting EGFR and/or HER2, comprising: administering a therapeutically effective amount of the compound represented by Chemical Formula 1, 2, 2a, 2b, or 3, the stereoisomer thereof, or the pharmaceutically acceptable salt thereof to a subject in need thereof.

The term “therapeutically effective amount” as used herein refers to an amount of the compound represented by Chemical Formula 1, 2, 2a, 2b, or 3 that is effective for the treatment or prevention of EGFR- and/or HER2-related diseases. Specifically, “therapeutically effective amount” indicates an amount sufficient to treat a disease with a reasonable benefit/risk ratio applicable to medical treatment, and the effective dose level may be determined depending on factors including the subject type and severity, age, sex, type of disease, drug activity, drug sensitivity, administration time, administration route and excretion rate, treatment period, drugs used at the same time, and other factors well-known in medical fields. The pharmaceutical composition of the present invention may be administered as an individual therapeutic agent or may be administered in combination with other therapeutic agents, and may be administered sequentially or simultaneously with commercially available therapeutic agents. In addition, the pharmaceutical composition of the present invention may be administered in a single dose or multiple doses. It is important to administer the minimum amount capable of obtaining the maximum effect without side effects in consideration of all of the above factors, and the amount may be readily determined by those skilled in the art. The dosage of the pharmaceutical composition of the present invention may be determined by a medical specialist according to various factors such as the patient's condition, age, sex, complications, and the like. Since the active ingredient of the pharmaceutical composition of the present invention has excellent safety, it may be used at a dose higher than the determined dosage.

As used herein, term “prevention” refers to any action that suppresses or delays the occurrence, spread, and recurrence of the disease by administering the compound, and term “treatment” refers to any action in which the symptoms of the disease are improved or beneficially changed by administration of the compound.

Further, according to an embodiment of the present invention, the pharmaceutical composition may further comprise a pharmaceutically acceptable carrier, diluent, or excipient. In an embodiment, the present invention provides a pharmaceutical composition comprising the compound represented by Chemical Formula 1, 2, 2a, 2b, or 3, or the pharmaceutically acceptable salt or the stereoisomer thereof, and a pharmaceutically acceptable additive.

Examples of additives used in the pharmaceutical composition may include sweeteners, binders, solvents, solubilizers, wetting agents, emulsifiers, isotonic agents, absorbents, disintegrants, antioxidants, preservatives, lubricants, fillers, flavoring agents, and the like. For example, the additives may include lactose, dextrose, sucrose, mannitol, sorbitol, cellulose, glycine, silica, talc, stearic acid, stearin, magnesium stearate, magnesium aluminosilicate, starch, gelatin, gum tragacanth, alginic acid, sodium alginate, methylcellulose, sodium carboxymethylcellulose, agar, water, ethanol, polyethylene glycol, polyvinylpyrrolidone, sodium chloride, calcium chloride, orange essence, strawberry essence, vanilla flavor, and the like.

The pharmaceutical composition may be combined in various formulation forms for oral administration (for example, tablets, pills, powders, capsules, syrups or emulsions) or parenteral administration (for example, intramuscular, intravenous or subcutaneous injection).

For example, the pharmaceutical composition may be combined into preparations for oral administration, and additives used at this time may include cellulose, calcium silicate, corn starch, lactose, sucrose, dextrose, calcium phosphate, stearic acid, magnesium stearate, calcium stearate, gelatin, talc, surfactants, suspending agents, emulsifiers, diluents, and the like. Specifically, solid preparations for oral administration may include tablets, pills, powders, granules, capsules, and the like, and these solid preparations may be formulated by mixing at least one excipient in the composition, such as starch, calcium carbonate, sucrose, lactose, gelatin, etc. In addition, lubricants such as magnesium stearate and talc may be used in addition to simple excipients. Further, examples of liquid preparations for oral administration may include suspensions, emulsions, syrups, and the like. Various excipients such as wetting agents, sweeteners, aromatics, preservatives, and the like, may be included in addition to water and liquid paraffin that are commonly used simple diluents.

Further, preparations for parenteral administration may include sterilized aqueous solutions, non-aqueous solutions, suspensions, emulsions, freeze-dried preparations, and suppositories. As the non-aqueous solvents and the suspensions, propylene glycol, polyethylene glycol, vegetable oils such as olive oil, and injectable esters such as ethyl oleate, and the like, may be used. As the base of the suppository, witepsol, macrogol, Tween 61, cacao butter, laurin butter, glycerogelatin and the like, may be used. Meanwhile, conventional additives such as solubilizers, isotonic agents, suspending agents, emulsifiers, stabilizers, preservatives, and the like, may be contained in the injection.

Further, the pharmaceutical composition may have a synergistic effect of active ingredients by being prepared as a complex formulation with other active agents.

Matters mentioned in the uses, compositions and treatment methods of the present invention are applied equally except to the extent that they are inconsistent with each other.

### Advantageous Effects of Invention

The heteroaryl derivative compound of the present invention exhibits excellent inhibitory activity against EGFR and/or HER2, and thus may be usefully employed for the treatment or prevention of EGFR- and/or HER2-related diseases.

### BEST MODE FOR CARRYING OUT THE INVENTION

Hereinafter, the present invention will be described in detail by Examples and Experimental Examples. However, the following Examples and Experimental Examples are only provided to illustrate the present invention, and the scope of the present invention is not limited to these Examples.

### <Analysis and Purification Conditions>

Compounds synthesized in Examples of the present invention were purified under the following HPLC and MPLC conditions or were subjected to structural analysis through NMR.

### 1. HPLC and MPLC

### HPLC Conditions for Analysis (ACQUITY UPLC H-Class Core System)

Waters UPLC system (ACQUITY UPLC PDA Detector) equipped with a mass QDa detector from Waters was used. Waters' ACQUITY UPLC® BEH C18 (1.7 μm, 2.1×50 mm) column was used, and the column temperature was set to 30° C.

Water containing 0.1% formic acid was used as mobile phase A, and acetonitrile containing 0.1% formic acid was used as mobile phase B.

Gradient condition (10-100% B for 3 minutes, flow rate=0.6 ml/min)

### Preparative HPLC System for Purification (Preparative-Liquid Chromatography UV Spectrometry)

The ACCQPrep HP150 equipment manufactured by Teledyne ISCO Inc., was used. Waters' XTERRA® Prep RP18 OBD™ (10 μm, 30×300 mm) column was used, and the column temperature was set to room temperature.

Gradient condition (10-100% B for 120 minutes, flow rate=42 ml/min)

### Medium Pressure Liquid Chromatography (MPLC) for Purification

Medium pressure liquid chromatography was performed using Teledyne ISCO's CombiFlash Rf+UV.

### 2. NMR Analysis

NMR analysis was performed using AVANCE III 400 or AVANCE III 400 HD manufactured by Bruker, and data were expressed as parts per million (δ) (ppm).

Commercial reagents used were used without further purification. In the present invention, ambient temperature or room temperature refers to a temperature of about 5° C. to 40° C., as an example, 10° C. to 30° C., and as another example, 20° C. to 27° C., and is not strictly limited to the above range. Concentration under reduced pressure or solvent distillation was performed using a rotary evaporator.

### <Preparation Example 1> Preparation of (S)-3-phenylisoxazolidine

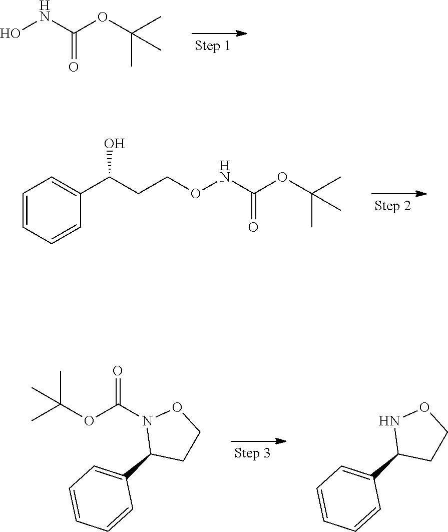

### Step 1: Preparation of tert-butyl (R)-(3-hydroxy-3-phenylpropoxy)carbamate

tert-butyl hydroxycarbamate (7.8 g, 58.6 mmol) was dissolved in dimethylformamide (DMF; 140 ml), then sodium hydride (2.58 g, 64.5 mmol) was added at 0° C., and the reaction mixture was reacted for 30 minutes. Next, (R)-3-chloro-1-phenylpropan-1-ol (5 g, 29.3 mmol) dissolved in dimethylformamide (10 ml) was slowly added dropwise at 0° C. for 10 minutes, and stirred at room temperature for 72 hours. The reaction was terminated by adding an aqueous ammonium chloride solution to the reaction mixture, and organic substances were extracted using ethyl acetate and brine. The organic layer was dried over sodium sulfate and concentrated under reduced pressure. The concentrate was purified by medium pressure liquid chromatography (ethyl acetate/n-hexane) to obtain the target compound (2.8 g, 68%).

MS (m/z): 150.17 [M+H]⁺, UPLC r.t. (min): 1.51

### Step 2: Preparation of tert-butyl (S)-3-phenylisoxazolidin-2-carboxylate

tert-butyl (R)-(3-hydroxy-3-phenylpropoxy)carbamate (2.55 g, 9.54 mmol) obtained in Step 1 above and triethylamine (3.13 ml, 22.44 mmol) were mixed with dichloromethane (250 ml) and then cooled to 0° C. Then, methanesulfonyl chloride (1 ml, 13 mmol) was added dropwise and reacted at 0° C. for 2 hours. Organic materials were extracted from the reaction mixture with brine and dichloromethane. The organic layer was dried over sodium sulfate and concentrated under reduced pressure to obtain the target compound, which was used in the next reaction without purification.

MS (m/z): 194.13 [M+H]⁺, UPLC r.t. (min): 1.69

### Step 3: Preparation of (S)-3-phenylisoxazolidine

tert-butyl (S)-3-phenylisoxazolidine-2-carboxylate (2.3 g) obtained in Step 2 above was dissolved in dichloromethane (DCM; 90 ml), then trifluoroacetic acid (14 ml) was added, and the reaction mixture was reacted at room temperature for 1 hour. The reaction mixture was neutralized with an aqueous sodium bicarbonate solution and the organic layer was extracted. The organic layer was dried over sodium sulfate and concentrated under reduced pressure. The concentrate was purified by medium pressure liquid chromatography (tetrahydrofuran/n-hexane) to obtain the target compound (1.3 g, 94%).

MS (m/z): 150.08 [M+H]⁺, UPLC r.t. (min): 0.72

### <Preparation Example 2> Preparation of (R)-3-phenylisoxazolidine

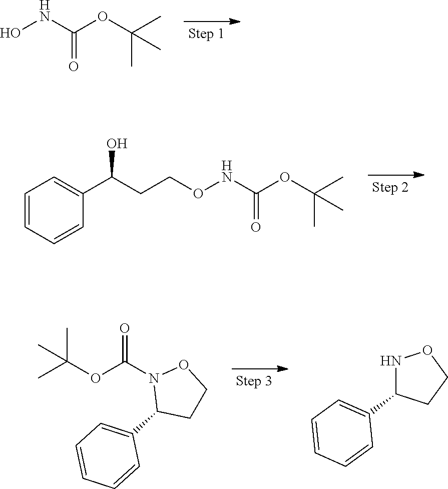

A compound of Preparation Example 2 was prepared in a similar manner to Preparation Example 1 above, and was used to synthesize compounds according to Examples shown in Table 1 below.

MS (m/z): 150.08 [M+H]⁺, UPLC r.t. (min): 0.72

### <Preparation Example 3> Preparation of (R)-3-(3-fluorophenyl)isoxazolidine

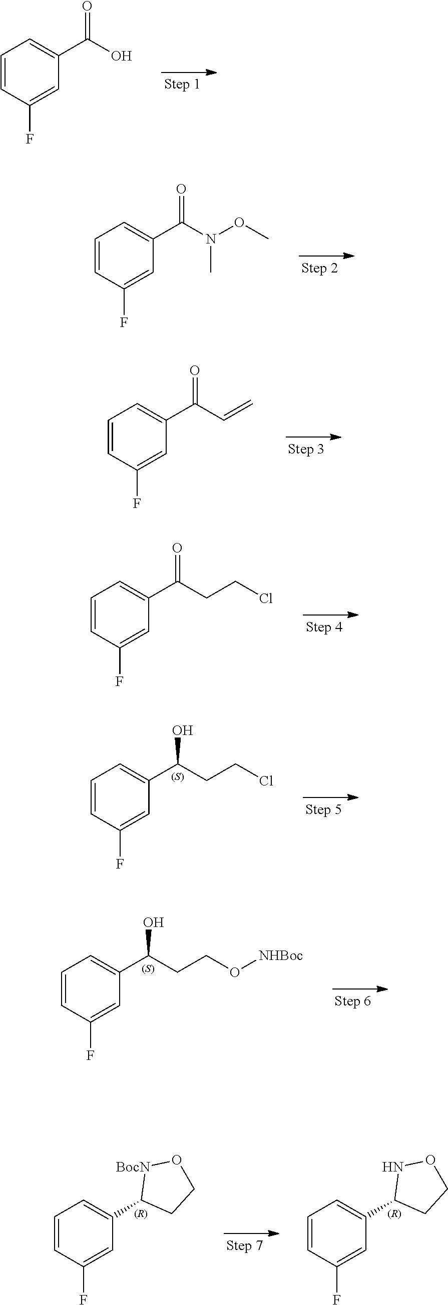

### Step 1: Preparation of 3-fluoro-N-methoxy-N-methylbenzamide

3-Fluorobenzoic acid (90 g, 642.35 mmol, 1 eq.) was dissolved in pyridine (150 mL), and then N-methoxy methanamine (75.19 g, 770.81 mmol, 1.2 eq., HCl) was added. Then, 1-ethyl-3-(3-dimethylaminopropyl) carbodiimide (EDCI; 147.77 g, 770.81 mmol, 1.2 eq.) was added at 15° C. The reaction mixture was stirred at 50° C. for 30 minutes. As a result of TLC analysis (petroleum ether (PE):ethyl acetate (EA)=3:1), all starting materials disappeared, and a new spot with low polarity was detected. The pyridine solvent was removed by concentration under reduced pressure, and the organic layer was extracted using dichloromethane (500 mL), hydrochloric acid (500 mL, 2N), and brine (200 mL). The organic layer was dried over sodium sulfate and concentrated under reduced pressure to obtain the target compound (110 g, 600.50 mmol, 93.49% yield) as a yellow oil.

¹H NMR (400 MHZ, CHLOROFORM-d) δ 7.47-7.40 (m, 1H), 7.39-7.38 (m, 2H), 7.14-7.13 (m, 1H), 3.54 (s, 3H), 3.45 (s, 3H).

### Step 2: Preparation of 1-(3-fluorophenyl)prop-2-en-1-one

3-Fluoro-N-methoxy-N-methyl-benzamide (110 g, 600.50 mmol, 1 eq.) obtained in Step 1 above was dissolved in tetrahydrofuran (THF; 1 L), and then bromo (vinyl) magnesium (1M, 630.53 mL, 1.05 eq.) was added dropwise at 0° C. Then, the reaction mixture was stirred at 0° C. for 30 minutes. As a result of TLC analysis (petroleum ether (PE):ethyl acetate (EA)=4:1), all starting materials disappeared, and a new spot with low polarity was detected. The reaction was terminated by adding hydrochloric acid (4N, 500 mL), and then the organic layer was extracted using methyl tert-butyl ether (MTBE; 2000 mL) and brine (500 mL). The organic layer was dried over sodium sulfate and concentrated under reduced pressure. The concentrated compound was purified using chromatography (petroleum ether/ethyl acetate=30/1) to obtain the target compound (80 g, 532.80 mmol, 88.73% yield) as a yellow oil.

¹H NMR (400 MHZ, CHLOROFORM-d) δ 7.65 (m, 1H), 7.58-7.52 (m, 1H), 7.39 (m, 1H), 7.24-7.17 (m, 1H), 7.04 (dd, J=17.2, 10.4 Hz, 1H), 6.39 (dd, J=17.2, 1.6 Hz, 1H), 5.90 (dd, J=10.4, 1.6 Hz, 1H).

### Step 3: Preparation of 3-chloro-1-(3-fluorophenyl)propan-1-one

1-(3-Fluorophenyl) prop-2-en-1-one (71 g, 472.86 mmol, 1.0 eq.) obtained in Step 2 above was dissolved in dichloromethane (71 mL) and then HCl/dioxane (4M, 295.54 mL, 2.5 eq.) was added at 0° C. Then, the reaction mixture was stirred at 15° C. for 1.5 hours. As a result of TLC analysis (petroleum ether (PE):ethyl acetate (EA)=10:1), all starting materials disappeared, and the target compound was detected. The reaction mixture was concentrated under reduced pressure, and dichloromethane (450 mL) and water (200 mL*5) were added to extract the organic layer. The organic layer was dried over sodium sulfate and concentrated under reduced pressure to obtain the target compound (73 g, 391.19 mmol, 82.73% yield) as a yellow solid.

¹H NMR (400 MHZ, CHLOROFORM-d) δ 7.78-7.72 (m, 1H), 7.69-7.60 (m, 1H), 7.53-7.44 (m, 1H), 7.37-7.24 (m, 1H), 3.93 (t, J=6.8 Hz, 2H), 3.46 (t, J=6.8 Hz, 2H).

### Step 4: Preparation of (S)-3-chloro-1-(3-fluorophenyl)propan-1-ol

(3aR)-1-Methyl-3,3-diphenyl-3a,4,5,6-tetrahydropyrrolo[1,2-c][1,3,2]oxazaborole (1M, 32.15 mL, 0.1 eq.) was dissolved in tetrahydrofuran (THF; 1.2 L), and then borane tetrahydrofuran (BH₃·THF; 1M, 186.48 mL, 0.6 eq.) was added dropwise under a nitrogen atmosphere at 0° C. The reaction mixture was stirred at 0° C. for 30 minutes. Then, 3-chloro-1-(3-fluorophenyl)propan-1-one (60 g, 309.02 mmol, 1 eq.) obtained in Step 3 and diluted in tetrahydrofuran was added dropwise to the reaction mixture at 0° C. The reaction mixture was stirred at 0° C. for 30 minutes. As a result of TLC analysis (petroleum ether (PE):ethyl acetate (EA)=5:1), all starting materials disappeared, and the target compound spot was detected. The reaction was terminated by adding methanol (100 mL) at 0° C., and then the solvent was evaporated under reduced pressure. The organic layer was extracted from the concentrated compound using dichloromethane (100 mL*3) and ammonium chloride (NH₄Cl) solution (300 mL). The organic layer was dried over sodium sulfate and concentrated under reduced pressure. The concentrated compound was purified using silica gel chromatography (petroleum ether (PE):ethyl acetate (EA)=50:1 to 5:1) to obtain the target compound (140 g, 664.2 mmol, 71.65% yield, 89.49% purity, 65.5% e.e) as a colorless oil.

¹H NMR (400 MHZ, CHLOROFORM-d) δ 7.33 (m, 1H), 7.16-7.07 (m, 2H), 7.02-6.96 (m, 1H), 4.96 (m, 1H), 3.75 (m, 1H), 3.57 (m, 1H), 2.26-2.15 (m, 2H).

### Step 5: Preparation of tert-butyl (S)-(3-(3-fluorophenyl)-3-hydroxypropoxy)carbamate

tert-butyl hydroxycarbamate (50.4 g, 378.52 mmol, 1.05 eq.) was dissolved in dimethylformamide (500 mL), and sodium hydride (NaH; 15.86 g, 396.55 mmol, 60% purity, 1.1 eq.) was dissolved in nitrogen atmosphere at 0° C. The reaction mixture was stirred at 10° C. for 1 hour, then (S)-3-chloro-1-(3-fluorophenyl)propan-1-ol (68 g, 360.5 mmol, 1 eq.) obtained in Step 4 above and diluted in dimethylformamide (180 mL) was added dropwise at 0° C., and the reaction mixture was stirred at 10° C. for 16 hours. As a result of TLC analysis (petroleum ether (PE):ethyl acetate (EA)=2:1), all starting materials disappeared, and the target compound was detected. The reaction was terminated by adding an aqueous ammonium chloride solution (3 L), and then the organic layer was extracted using ethyl acetate (2000 mL) and brine (2000 mL). The organic layer was dried over sodium sulfate and concentrated under reduced pressure to obtain the target compound (176 g, 616.87 mmol, 85.56% yield) as a bright yellow solid.

¹H NMR (400 MHZ, CHLOROFORM-d) δ 7.67-7.64 (m, 1H), 7.23-7.17 (m, 1H), 7.08-7.03 (m, 2H), 6.88-6.81 (m, 1H), 4.99-4.84 (m, 1H), 4.02-3.97 (m, 1H), 3.96-3.89 (m, 1H), 1.95-1.89 (m, 1H), 1.88-1.78 (m, 1H), 1.42-1.39 (m, 9H).

### Step 6: Preparation of tert-butyl (R)-3-(3-fluorophenyl)isoxazolidin-2-carboxylate

tert-butyl (S)-(3-(3-fluorophenyl)-3-hydroxypropoxy)carbamate (88 g, 308.44 mmol, 1 eq.) obtained in Step 5 above and triethylamine (93.63 g, 925.31 mmol, 128.79 mL, 3 eq.) were dissolved in dichloromethane (1 L), and then methanesulfonic anhydride (80.59 g, 462.65 mmol, 1.5 eq.) was added slowly at 0° C. The reaction mixture was stirred at 20° C. for 12 hours. As a result of TLC analysis (petroleum ether (PE):ethyl acetate (EA)=3:1), all starting materials disappeared, and a new spot was detected. The reaction was terminated by adding water (2000 mL), and the organic layer was extracted using dichloromethane (DCM; 200 mL*3). The organic layer was dried over sodium sulfate and concentrated under reduced pressure. The concentrated compound was purified by chromatography (petroleum ether (PE):ethyl acetate (EA)=50:1 to 5:1) to extract the target compound (88 g) with an e.e value of 82.5%. The target compound was purified through SFC (column: DAICEL CHIRALPAK AD (250 mm*50 mm, 10 μm); mobile phase: [Neu-MeOH]; B %: 15%˜15%, 3.4 min; 380 min) to obtain the target compound (51 g, 189.66 mmol, 30.74% yield, 99.4% purity) as a white solid.

The purity of the optical isomer of tert-butyl (R)-3-(3-fluorophenyl)isoxazolidin-2-carboxylate obtained in Step 6 was analyzed under the SFC conditions below.
Instrument: CAS-WH-ANA-SFC-C(SHIMADZU LC-30ADsf)
Column: Amycoat 50×4.6 mm I.D., 3 μm
Mobile phase: Phase A for CO₂, and Phase B for MeOH (0.05% DEA);
Gradient elution: MeOH (0.05% DEA) in CO₂ from 5% to 40%
Flow rate: 3 mL/min; Detector: PDA;
Column Temp: 35° C.; Back Pressure: 100 Bar

When the optical isomer of tert-butyl (R)-3-(3-fluorophenyl)isoxazolidin-2-carboxylate obtained in Step 6 has low purity, the product was purified under the SFC conditions below to obtain the desired optical isomer as a yellow liquid.
Column: DAICEL CHIRALPAK AD-H (250 mm×30 mm, 5 μm);
Mobile phase: [0.1% NH₃H₂O MeOH]; B %: 15%˜15%, 3.8 min; 600 min

### Step 7: Preparation of (R)-3-(3-fluorophenyl)isoxazolidine

tert-butyl (R)-3-(3-fluorophenyl)isoxazolidin-2-carboxylate (50 g, 185.94 mmol, 1 eq.) obtained in Step 6 above was dissolved in ethyl acetate (EA; 200 mL), and then HCl/EtOAc (4M, 300 mL, 6.45 eq.) was added at 0° C. Then, the reaction mixture was stirred at 10° C. for 1 hour. As a result of LCMS analysis, all starting materials disappeared, and to obtain a solid, the reaction product was concentrated under reduced pressure to obtain the target compound (32 g, 150.26 mmol, 80.81% yield, 95.62% purity, 100% e.e. HCl) as a white solid.

MS: m/z 168.2 [M+H]⁺

¹H NMR (400 MHZ, DMSO-d₆) δ 7.53-7.43 (m, 2H), 7.39 (d, J=7.8 Hz, 1H), 7.30-7.23 (m, 1H), 5.01 (t, J=8.0 Hz, 1H), 4.47 (m, 1H), 4.27 (m, 1H), 2.87 (m, 1H), 2.62-2.52 (m, 1H).

In Step 7 above, the following conditions were used to purify or analyze optical isomers of the compounds.
Instrument: CAS-WH-ANA-SFC-C(SHIMADZU LC-30ADsf)
Column: Chiralpak AY-3 50×4.6 mm I.D., 3 μm;
Mobile phase: Phase A for CO₂, and Phase B for IPA (0.05% DEA);
Gradient elution: B in A from 5% to 40%;
Flow rate: 3 mL/min; Detector: PDA;
Column Temp: 35° C.; Back Pressure: 100 Bar

### Preparation Examples 4 to 9

Compounds of Preparation Examples 4 to 9 below were prepared in a similar manner to Preparation Examples 1 to 3 above, and Compounds according to Examples of the present invention were prepared using the compounds of Preparation Examples 1 to 9.

### <Preparation Example 4> Preparation of (R)-3-(3,5-difluorophenyl)isoxazolidine

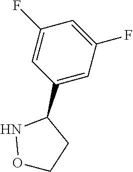 [SMILES: Fc1cc(F)cc([C@H]2CCON2)c1]

¹H NMR (400 MHZ, DMSO-d₆) δ 7.36-7.27 (m, 3H), 5.04-4.98 (t, J=7.6 Hz, 1H), 4.46-4.36 (m, 1H), 4.25-4.19 (dd, J=7.6, 15.2 Hz, 1H), 2.90-2.78 (m, 1H), 2.56-2.51 (m, 1H).

### <Preparation Example 5> Preparation of (R)-3-(2,5-difluorophenyl)isoxazolidine

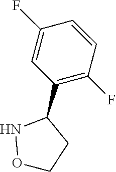 [SMILES: Fc1ccc(F)c([C@H]2CCON2)c1]

### <Preparation Example 6> Preparation of (R)-3-(4-fluorophenyl)isoxazolidine

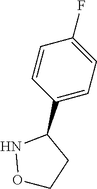 [SMILES: Fc1ccc([C@H]2CCON2)cc1]

### <Preparation Example 7> Preparation of (R)-3-(2,4-difluorophenyl)isoxazolidine

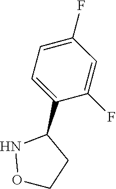 [SMILES: Fc1ccc([C@H]2CCON2)c(F)c1]

¹H NMR (400 MHZ, CHLOROFORM-d) δ 7.52-7.47 (m, 1H), 6.87-6.75 (m, 2H), 5.30 (s, 1H), 4.71-4.68 (m, 1H), 4.09-4.04 (m, 1H), 3.91-3.85 (m, 1H), 2.73-2.64 (3, 1H), 2.24-2.20 (m, 1H).

### <Preparation Example 8> Preparation of (R)-3-(3,4-difluorophenyl)isoxazolidine

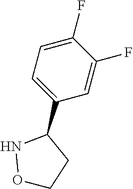 [SMILES: Fc1ccc([C@H]2CCON2)cc1F]

¹H NMR (400 MHZ, CHLOROFORM-d) δ 7.24-7.19 (m, 1H), 7.12-7.06 (m, 2H), 5.24 (s, 1H), 4.46 (dd, J1=8.4 Hz, J2=5.6 Hz, 1H), 4.05 (dt, J1=8.0 Hz, J2=5.2 Hz, 1H), 3.91-3.85 (m, 1H), 2.70-2.61 (m, 1H), 2.25-2.17 (m, 1H).

### <Preparation Example 9> Preparation of (R)-3-(2,3-difluorophenyl)isoxazolidine

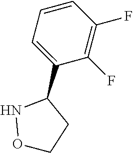 [SMILES: Fc1cccc([C@H]2CCON2)c1F]

¹H NMR (CHLOROFORM-d, 400 MHZ) δ 7.29-7.27 (m, 1H), 7.06-7.02 (m, 2H), 5.44 (br s, 1H), 4.75 (dd, J1=4.4 Hz, J2=8.4 Hz, 1H), 4.08 (dt, J1=5.2 Hz, J2=8.0 Hz, 1H), 3.86 (q, J=8.0 Hz, 1H), 2.76-2.66 (m, 1H), 2.27-2.19 (m, 1H).

### <Example 1> Preparation of (R)-6-(3-(2,3-difluorophenyl)isoxazolidin-2-yl)-N-(5-(3-fluoro-1-methyl-1H-pyrazol-4-yl)-4-(9-(1-fluoro-2-methylpropan-2-yl)-3,9-di azaspiro[5.5]undecan-3-yl)-2-methoxyphenyl)pyrimidin-4-amine

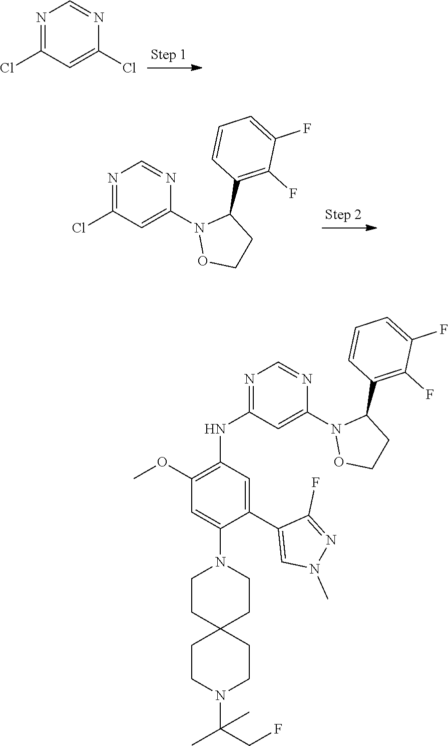 [SMILES: Clc1cc(Cl)ncn1 ; Fc1cccc([C@H]2CCON2c2cc(Cl)ncn2)c1F ; COc1cc(N2CCC3(CC2)CCN(C(C)(C)CF)CC3)c(-c2cn(C)nc2F)cc1Nc1cc(N2OCC[C@@H]2c2cccc(F)c2F)ncn1]

### Step 1: Preparation of (R)-2-(6-chloropyrimidin-4-yl)-3-(2,3-difluorophenyl)isoxazolidine

4,6-Dichloropyrimidine (3 g, 1 eq.) and (R)-3-(2,3-difluorophenyl)isoxazolidine (4.1 g, 1.1 eq.) were dissolved in ethanol (20 ml). Then, N,N-diisopropylethylamine (DIPEA; 7.74 mL, 2.2 eq.) was added. The reaction solution was stirred at 100° C. for 3 hours. After completion of the reaction, the reaction solution was extracted using ethyl acetate and water. The collected organic layer was washed with brine, dried over anhydrous sodium sulfate, concentrated under reduced pressure, and purified by MPLC (ethyl acetate/hexane) to obtain the target compound (5.94 g, 99% yield) as a clear liquid.

### Step 2: Preparation of (R)-6-(3-(2,3-difluorophenyl)isoxazolidin-2-yl)-N-(5-(3-fluoro-1-methyl-1H-pyrazol-4-yl)-4-(9-(1-fluoro-2-methylpropan-2-yl)-3,9-diaza spiro[5.5]undecan-3-yl)-2-methoxyphenyl)pyrimidin-4-amine

(R)-2-(6-Chloropyrimidin-4-yl)-3-(2,3-difluorophenyl)isoxazolidine (50 mg, 1 eq.) obtained in Step 1 above, 5-(3-fluoro-1-methyl-1H-pyrazol-4-yl)-4-(9-(1-fluoro-2-methylpropan-2-yl)-3,9-diazaspiro[5.5]undecan-3-yl)-2-methoxyaniline (150 mg, 2 eq.), and cesium carbonate (109 mg, 2 eq.) were added and dissolved in 1,4-dioxane (1.7 ml), and then gases were removed by sonication for 5 minutes under nitrogen. Palladium acetate (Pd(OAc)₂; 7.54 mg, 0.2 eq.) and BINAP (20.92 mg, 0.2 eq.) were added to the reaction mixture, and then stirred at 90° C. for 6 hours. After completion of the reaction, the reaction mixture was filtered through celite and washed with dichloromethane. The obtained filtrate was concentrated and purified by Prep-HPLC to obtain the target compound (20 mg, 16.8%).

¹H NMR (400 MHZ, Chloroform-d) δ 8.31 (d, J=1.0 Hz, 1H), 7.86 (d, J=2.0 Hz, 1H), 7.73 (s, 1H), 7.37-7.29 (m, 1H), 7.11-7.00 (m, 2H), 6.91 (s, 1H), 6.73 (s, 1H), 6.65 (d, J=1.0 Hz, 1H), 5.92 (dd, J=8.8, 4.8 Hz, 1H), 4.14 (td, J=8.1, 4.0 Hz, 1H), 3.94 (q, J=8.0 Hz, 1H), 3.88 (s, 3H), 3.82 (s, 3H), 2.84 (qt, J=11.3, 5.2 Hz, 4H), 2.56-2.49 (m, 4H), 2.47 (s, 1H), 2.42 (s, 1H), 2.37-2.25 (m, 2H), 1.59-1.52 (m, 8H), 1.39 (s, 3H), 1.34 (s, 3H).

### Examples 2 to 113

All compounds according to Examples of the present invention (Compounds of Examples 2 to 113) were prepared in a similar manner to Example 1 above, and the compound names, chemical structural formulas, NMR, and LCMS analysis results of respective compounds according to Examples above are summarized and shown in Table 1 below.

TABLE 1

| Ex- | | | |
|---|---|---|---|
| am- | | | |
| ple | Structure | Compound Name | ¹H NMR; MS [M + H] ⁺ |
| 1 | 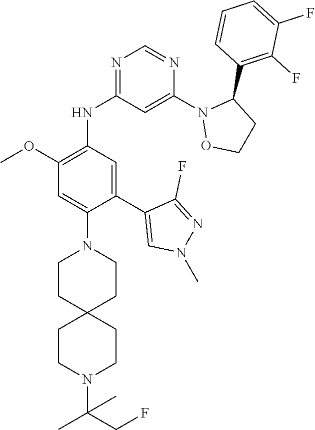 | (R)-6-(3-(2,3- difluorophenyl)isoxazolidin- 2-yl)-N-(5-(3-fluoro-1- methyl-1H-pyrazol-4-yl)-4- (9-(1-fluoro-2- methylpropan-2-yl)-3,9- diazaspiro[5.5]undecan-3- yl)-2- methoxyphenyl)pyrimidin-4- amine | ¹H NMR (400 MHz, Chloroform-d) δ 8.31 (d, J = 1.0 Hz, 1H), 7.86 (d, J = 2.0 Hz, 1H), 7.73 (s, 1H), 7.37-7.29 (m, 1H), 7.11-7.00 (m, 2H), 6.91 (s, 1H), 6.73 (s, 1H), 6.65 (d, J = 1.0 Hz, 1H), 5.92 (dd, J = 8.8, 4.8 Hz, 1H), 4.14 (td, J = 8.1, 4.0 Hz, 1H), 3.94 (q, J = 8.0 Hz, 1H), 3.88 (s, 3H), 3.82 (s, 3H), 2.84 (qt, J = 11.3, 5.2 Hz, 4H), 2.56-2.49 (m, 4H), 2.47 (s, 1H), 2.42 (s, 1H), 2.37-2.25 (m, 2H), 1.59-1.52 (m, 8H), 1.39 (s, 3H), 1.34 (s, 3H); 693.3 [M + H]⁺ |
| 2 | 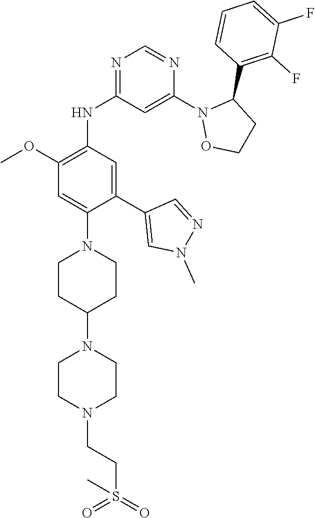 [SMILES: COc1cc(N2CCC(N3CCN(CCS(C)(=O)=O)CC3)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- (4-(4-(2- (methylsulfonyl)ethyl)pipera- zin-1-yl)piperidin-1- yl)phenyl)pyrimidin-4-amine | 738.3 [M + H]⁺ |
| 3 | 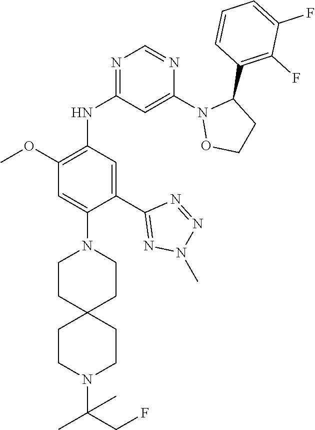 | (R)-6-(3-(2,3- difluorophenyl)isoxazolidin- 2-yl)-N-(4-(9-(1-fluoro-2- methylpropan-2-yl)-3,9- diazaspiro[5.5]undecan-3- yl)-2-methoxy-5-(2-methyl- 2H-tetrazol-5- yl)phenyl)pyrimidin-4-amine | 709.3 [M + H]⁺ |
| 4 | 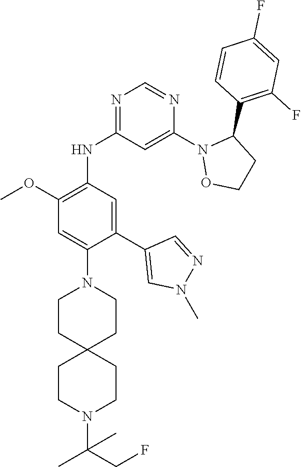 [SMILES: COc1cc(N2CCC3(CC2)CCN(C(C)(C)CF)CC3)c(-c2cnn(C)c2)cc1Nc1cc(N2OCC[C@@H]2c2ccc(F)cc2F)ncn1] | (R)-6-(3-(2,4- difluorophenyl)isoxazolidin- 2-yl)- N-(4-(9-(1-fluoro-2- methylpropan-2-yl)-3,9- diazaspiro[5.5]undecan-3- yl)-2-methoxy-5-(1-methyl- 1H-pyrazol-4- yl)phenyl)pyrimidin-4-amine | 691.3 [M + H]⁺ |
| 5 | 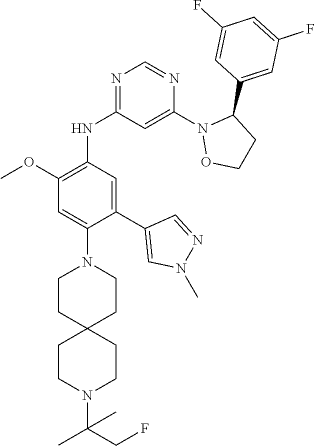 [SMILES: COc1cc(N2CCC3(CC2)CCN(C(C)(C)CF)CC3)c(-c2cnn(C)c2)cc1Nc1cc(N2OCC[C@@H]2c2cc(F)cc(F)c2)ncn1] | (R)-6-(3-(3,5- difluorophenyl)isoxazolidin- 2-yl)-N-(4-(9-(1-fluoro-2- methylpropan-2-yl)-3,9- diazaspiro[5.5]undecan-3- yl)-2-methoxy-5-(1-methyl- 1H-pyrazol-4- yl)phenyl)pyrimidin-4-amine | 691.3 [M + H]⁺ |
| 6 | 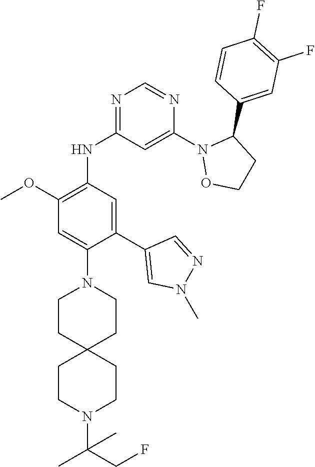 [SMILES: COc1cc(N2CCC3(CC2)CCN(C(C)(C)CF)CC3)c(-c2cnn(C)c2)cc1Nc1cc(N2OCC[C@@H]2c2ccc(F)c(F)c2)ncn1] | (R)-6-(3-(3,4- difluorophenyl)isoxazolidin- 2-yl)-N-(4-(9-(1-fluoro-2- methylpropan-2-yl)-3,9- diazaspiro[5.5]undecan-3- yl)-2-methoxy-5-(1-methyl- 1H-pyrazol-4- yl)phenyl)pyrimidin-4-amine | 691.3 [M + H]⁺ |
| 7 | 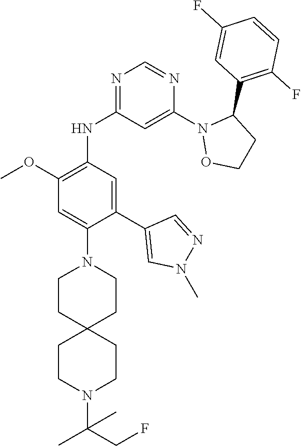 [SMILES: COc1cc(N2CCC3(CC2)CCN(C(C)(C)CF)CC3)c(-c2cnn(C)c2)cc1Nc1cc(N2OCC[C@@H]2c2cc(F)ccc2F)ncn1] | (R)-6-(3-(2,5- difluorophenyl)isoxazolidin- 2-yl)-N-(4-(9-(1-fluoro-2- methylpropan-2-yl)-3,9- diazaspiro[5.5]undecan-3- yl)-2-methoxy-5-(1-methyl- 1H-pyrazol-4- yl)phenyl)pyrimidin-4-amine | 691.3 [M + H]⁺ |
| 8 | 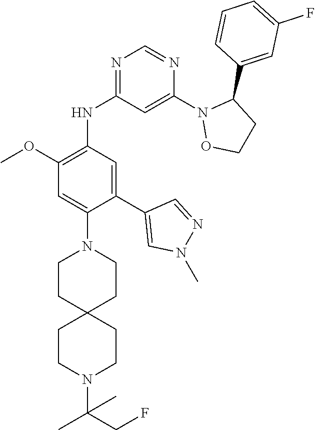 [SMILES: COc1cc(N2CCC3(CC2)CCN(C(C)(C)CF)CC3)c(-c2cnn(C)c2)cc1Nc1cc(N2OCC[C@@H]2c2cccc(F)c2)ncn1] | (R)-N-(4-(9-(1-fluoro-2- methylpropan-2-yl)-3,9- diazaspiro[5.5]undecan-3- yl)-2-methoxy-5-(1-methyl- 1H-pyrazol-4-yl)phenyl)-6- (3-(3- fluorophenyl)isoxazolidin-2- yl)pyrimidin-4-amine | 673.3 [M + H]⁺ |
| 9 | 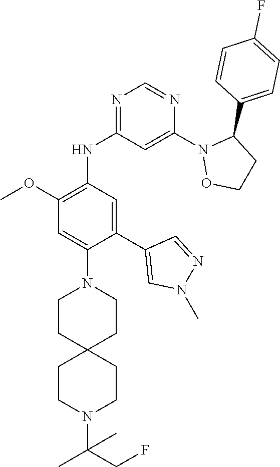 [SMILES: COc1cc(N2CCC3(CC2)CCN(C(C)(C)CF)CC3)c(-c2cnn(C)c2)cc1Nc1cc(N2OCC[C@@H]2c2ccc(F)cc2)ncn1] | (R)-N-(4-(9-(1-fluoro-2- methylpropan-2-yl)-3,9- diazaspiro[5.5]undecan-3- yl)-2-methoxy-5-(1-methyl- 1H-pyrazol-4-yl)phenyl)-6- (3-(4- fluorophenyl)isoxazolidin-2- yl)pyrimidin-4-amine | 673.3 [M + H]⁺ |
| 10 | 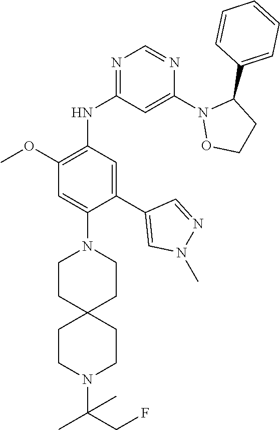 [SMILES: COc1cc(N2CCC3(CC2)CCN(C(C)(C)CF)CC3)c(-c2cnn(C)c2)cc1Nc1cc(N2OCC[C@@H]2c2ccccc2)ncn1] | (R)-N-(4-(9-(1-fluoro-2- methylpropan-2-yl)-3,9- diazaspiro[5.5]undecan-3- yl)-2-methoxy-5-(1-methyl- 1H-pyrazol-4-yl)phenyl)-6- (3-phenylisoxazolidin-2- yl)pyrimidin-4-amine | ¹H NMR (400 MHz, CDCl₃) δ 8.33 (s, 1H), 7.95 (s, 1H), 7.80 (s, 1H), 7.75 (s, 1H), 7.45 (d, J = 7.4 Hz, 2H), 7.34 (t, J = 7.6 Hz, 2H), 7.24 (d, J = 7.3 Hz, 1H), 6.86 (s, 1H), 6.72 (s, 1H), 6.49 (s, 1H), 5.69 (dd, J = 8.5, 4.5 Hz, 1H), 4.13 (ddd, J = 7.7, 6.1, 3.5 Hz, 1H), 3.94 (s, 3H), 3.91 (d, J = 8.0 Hz, 1H), 3.88 (s, 3H), 2.87-2.81 (m, 4H), 2.73 (ddd, J = 16.2, 7.8, 4.0 Hz, 1H), 2.43-2.37 (m, 1H), 1.77-1.25 (m, 20H); 655.5 [M + H]⁺ |
| 11 | 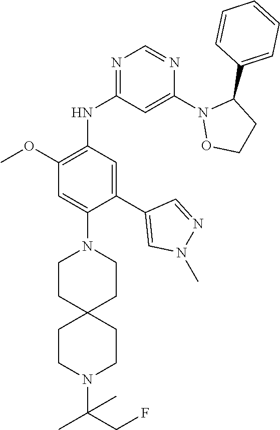 [SMILES: COc1cc(N2CCC3(CC2)CCN(C(C)(C)CF)CC3)c(-c2cnn(C)c2)cc1Nc1cc(N2OCC[C@@H]2c2ccccc2)ncn1] | (R)-6-(3-(2,3- difluorophenyl)isoxazolidin- 2-yl)-N-(4-(4-(1-(1-fluoro-2- methylpropan-2-yl)azetidin- 3-yl)piperidin-1-yl)-2- methoxy-5-(1-methyl-1H- pyrazol-4- yl)phenyl)pyrimidin-4-amine | 677.3 [M + H]⁺ |
| 12 | 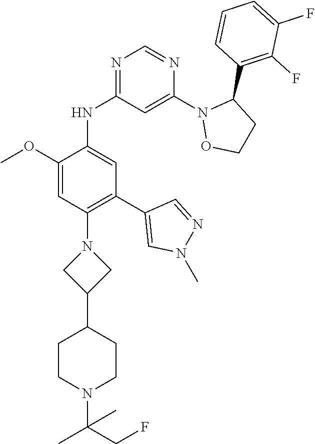 [SMILES: COc1cc(N2CC(C3CCN(C(C)(C)CF)CC3)C2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(4-(3-(1-(1-fluoro-2- methylpropan-2- yl)piperidin-4-yl)azetidin-1- yl)-2-methoxy-5-(1-methyl- 1H-pyrazol-4- yl)phenyl)pyrimidin-4-amine | 677.3 [M + H]⁺ |
| 13 | 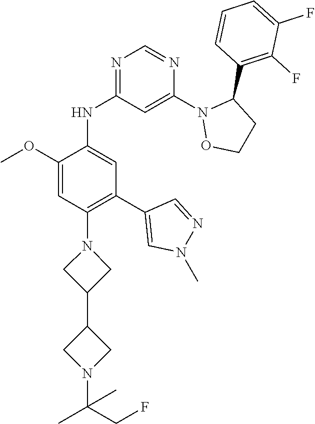 [SMILES: COc1cc(N2CC(C3CN(C(C)(C)CF)C3)C2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(4-(1′-(1-fluoro-2- methylpropan-2-yl)-[3,3′- biazetidin]-1-yl)-2-methoxy- 5-(1-methyl-1H-pyrazol-4- yl)phenyl)pyrimidin-4-amine | 649.3 [M + H]⁺ |
| 14 | 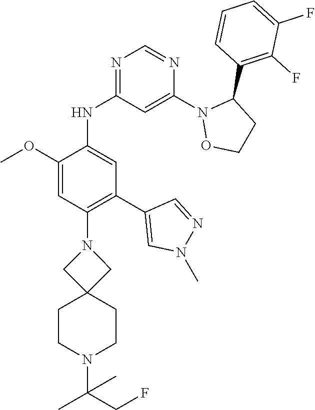 [SMILES: COc1cc(N2CC3(CCN(C(C)(C)CF)CC3)C2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(4-(7-(1-fluoro-2- methylpropan-2-yl)-2,7- diazaspiro[3.5]nonan-2-yl)- 2-methoxy-5-(1-methyl-1H- pyrazol-4- yl)phenyl)pyrimidin-4-amine | ¹H NMR (400 MHz, CDCl₃) δ 8.28 (s, 1H), 7.56 (s, 1H), 7.40 (s, 1H), 7.34 (s, 2H), 7.08- 7.02 (m, 2H), 6.63 (s, 1H), 6.39 (s, 1H), 6.16 (s, 1H), 5.91 (dd, J = 9.0, 4.7 Hz, 1H), 4.09 (td, J = 7.8, 4.2 Hz, 1H), 3.96 (s, 2H), 3.93 (s, 1H), 3.87 (s, 3H), 3.71 (s, 2H), 3.37 (s, 3H), 3.28 (s, 1H), 2.86-2.77 (m, 1H), 2.41 (t, J = 6.0 Hz, 5H), 2.33-2.26 (m, 1H), 1.75-1.71 (m, 4H), 1.37 (s, 3H), 1.32 (s, 3H).; 663.4 [M + H]⁺ |
| 15 | 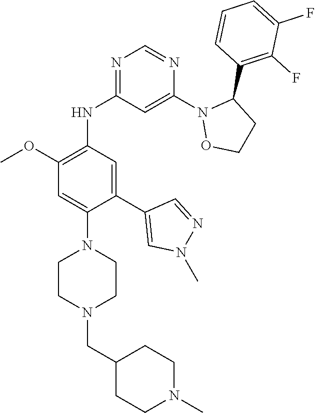 [SMILES: COc1cc(N2CCN(CC3CCN(C)CC3)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- (4-((1-methylpiperidin-4- yl)methyl)piperazin-1- yl)phenyl)pyrimidin-4-amine | 660.3 [M + H]⁺ |
| 16 | 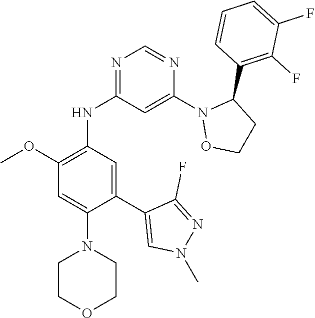 [SMILES: COc1cc(N2CCOCC2)c(-c2cn(C)nc2F)cc1Nc1cc(N2OCC[C@@H]2c2cccc(F)c2F)ncn1] | (R)-6-(3-(2,3- difluorophenyl)isoxazolidin- 2-yl)-N-(5-(3-fluoro-1- methyl-1H-pyrazol-4-yl)-2- methoxy-4- morpholinophenyl)pyrimidin- 4-amine | ¹H NMR (400 MHz, DMSO-d₆) δ 8.65 (s, 1H), 8.16 (s, 1H), 8.07 (d, J = 2.3 Hz, 1H), 7.62 (s, 1H), 7.40-7.25 (m, 2H), 7.25-7.14 (m, 1H), 6.82 (s, 1H), 6.38 (s, 1H), 5.81-5.67 (m, 1H), 4.18 (td, J = 7.9, 3.6 Hz, 1H), 3.85 (s, 3H), 3.84-3.82 (m, 1H), 3.78 (s, 3H), 3.68 (dd, J = 5.9, 3.2 Hz, 4H), 2.83 (q, J = 7.5, 6.0 Hz, 4H), 2.79 (dd, J = 6.2, 2.3 Hz, 1H), 2.27-2.14 (m, 1H).; 568.3 [M + H]⁺ |
| 17 | 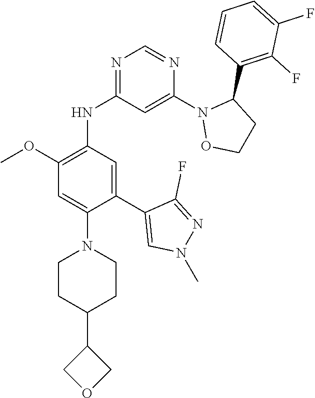 [SMILES: COc1cc(N2CCC(C3COC3)CC2)c(-c2cn(C)nc2F)cc1Nc1cc(N2OCC[C@@H]2c2cccc(F)c2F)ncn1] | (R)-6-(3-(2,3- difluorophenyl)isoxazolidin- 2-yl)-N-(5-(3-fluoro-1- methyl-1H-pyrazol-4-yl)-2- methoxy-4-(4-(oxetan-3- yl)piperidin-1- yl)phenyl)pyrimidin-4-amine | ¹H NMR (400 MHz, DMSO) δ 8.64 (s, 1H), 8.15 (d, J = 1.0 Hz, 1H), 8.00 (d, J = 2.2 Hz, 1H), 7.56 (s, 1H), 7.36 (ddd, J = 10.4, 6.3, 2.0 Hz, 1H), 7.30 (dd, J = 13.6, 6.2 Hz, 1H), 7.24- 7.16 (m, 1H), 6.81 (s, 1H), 6.34 (s, 1H), 5.76- 5.71 (m, 1H), 4.63 (dd, J = 7.9, 5.9 Hz, 2H), 4.36 (t, J = 6.2 Hz, 2H), 4.18 (td, J = 7.9, 3.6 Hz, 1H), 3.87-3.80 (m, 1H), 3.83 (s, 3H), 3.76 (s, 3H), 3.06 (d, J = 11.2 Hz, 2H), 2.85-2.74 (m, 2H), 2.59 (t, J = 11.8 Hz, 2H), 2.26-2.16 (m, 1H), 1.68 (d, J = 10.9 Hz, 1H), 1.60 (d, J = 12.9 Hz, 2H), 1.14 (q, J = 11.5 Hz, 2H).; 622.3 [M + H]⁺ |
| 18 | 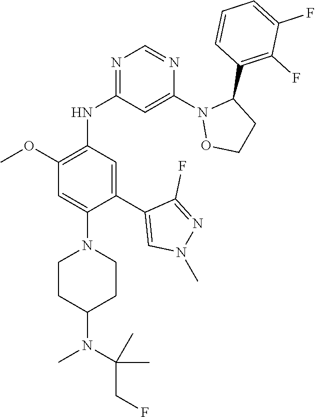 [SMILES: COc1cc(N2CCC(N(C)C(C)(C)CF)CC2)c(-c2cn(C)nc2F)cc1Nc1cc(N2OCC[C@@H]2c2cccc(F)c2F)ncn1] | (R)-6-(3-(2,3- difluorophenyl)isoxazolidin- 2-yl)-N-(5-(3-fluoro-1- methyl-1H-pyrazol-4-yl)-4- (4-((1-fluoro-2- methylpropan-2- yl)(methyl)amino)piperidin- 1-yl)-2- methoxyphenyl)pyrimidin-4- amine | 669.3 [M + H]⁺ |
| 19 | 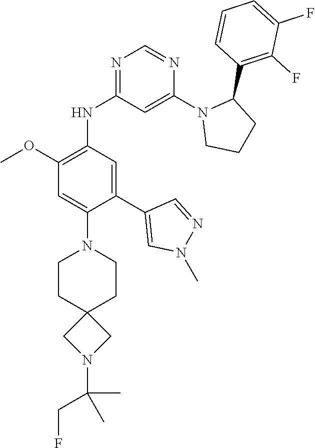 [SMILES: COc1cc(N2CCC3(CC2)CN(C(C)(C)CF)C3)c(-c2cnn(C)c2)cc1Nc1cc(N2CCC[C@@H]2c2cccc(F)c2F)ncn1] | (R)-6-(2-(2,3- difluorophenyl)pyrrolidin-1- yl)-N-(4-(2-(1-fluoro-2- methylpropan-2-yl)-2,7- diazaspiro[3.5]nonan-7-yl)- 2-methoxy-5-(1-methyl-1H- pyrazol-4- yl)phenyl)pyrimidin-4-amine | 661.3 [M + H]⁺ |
| 20 | 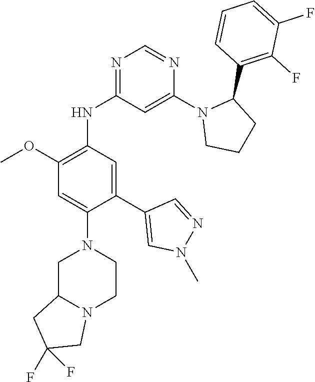 [SMILES: COc1cc(N2CCN3CC(F)(F)CC3C2)c(-c2cnn(C)c2)cc1Nc1cc(N2CCC[C@@H]2c2cccc(F)c2F)ncn1] | N-(4-(7,7- difluorohexahydropyrrolo[1, 2-a]pyrazin-2(1H)-yl)-2- methoxy-5-(1-methyl-1H- pyrazol-4-yl)phenyl)-6-((R)- 2-(2,3- difluorophenyl)pyrrolidin-1- yl)pyrimidin-4-amine | 623.3 [M + H]⁺ |
| 21 | 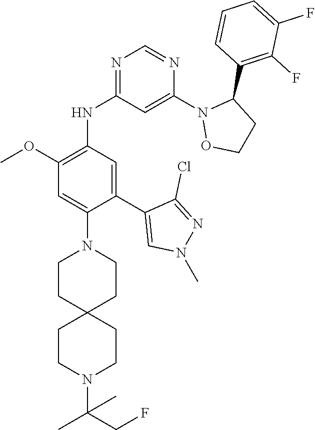 [SMILES: COc1cc(N2CCC3(CC2)CCN(C(C)(C)CF)CC3)c(-c2cn(C)nc2Cl)cc1Nc1cc(N2OCC[C@@H]2c2cccc(F)c2F)ncn1] | (R)-N-(5-(3-chloro-1- methyl-1H-pyrazol-4-yl)-4- (9-(1-fluoro-2- methylpropan-2-yl)-3,9- diazaspiro[5.5]undecan-3- yl)-2-methoxyphenyl)-6-(3- (2,3- difluorophenyl)isoxazolidin- 2-yl)pyrimidin-4-amine | 726.3 [M + H]⁺ |
| 22 | 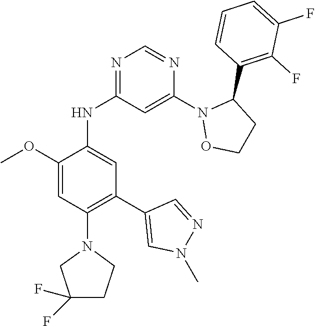 [SMILES: COc1cc(N2CCC(F)(F)C2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(4-(3,3- difluoropyrrolidin-1-yl)-2- methoxy-5-(1-methyl-1H- pyrazol-4- yl)phenyl)pyrimidin-4-amine | 570.3 [M + H]⁺ |
| 23 | 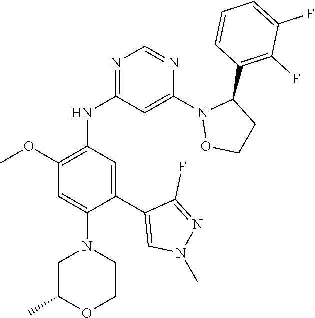 [SMILES: COc1cc(N2CCO[C@H](C)C2)c(-c2cn(C)nc2F)cc1Nc1cc(N2OCC[C@@H]2c2cccc(F)c2F)ncn1] | 6-((R)-3-(2,3- difluorophenyl)isoxazolidin- 2-yl)-N-(5-(3-fluoro-1- methyl-1H-pyrazol-4-yl)-2- methoxy-4-((R)-2- methylmorpholino)phenyl)py- rimidin-4-amine | 582.3 [M + H]⁺ |
| 24 | 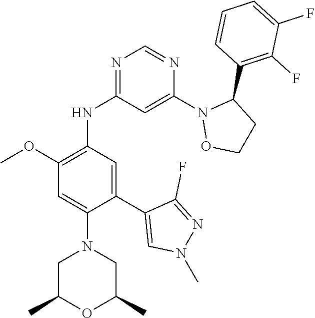 [SMILES: COc1cc(N2C[C@@H](C)O[C@@H](C)C2)c(-c2cn(C)nc2F)cc1Nc1cc(N2OCC[C@@H]2c2cccc(F)c2F)ncn1] | 6-((R)-3-(2,3- difluorophenyl)isoxazolidin- 2-yl)-N-(4-((2S,6R)-2,6- dimethylmorpholino)-5-(3- fluoro-1-methyl-1H-pyrazol- 4-yl)-2- methoxyphenyl)pyrimidin-4- amine | 596.3 [M + H]⁺ |
| 25 | 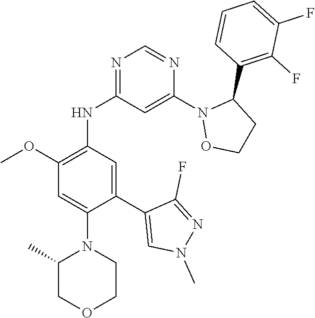 [SMILES: COc1cc(N2CCOC[C@@H]2C)c(-c2cn(C)nc2F)cc1Nc1cc(N2OCC[C@@H]2c2cccc(F)c2F)ncn1] | 6-((R)-3-(2,3- difluorophenyl)isoxazolidin- 2-yl)-N-(5-(3-fluoro-1- methyl-1H-pyrazol-4-yl)-2- methoxy-4-((S)-3- methylmorpholino)phenyl)py- rimidin-4-amine | 582.3 [M + H]⁺ |
| 26 | 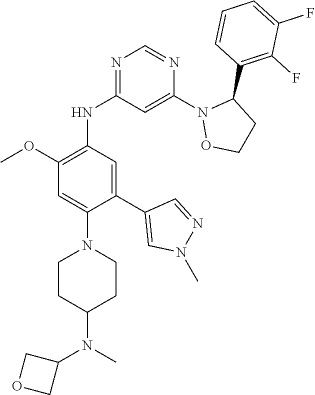 [SMILES: COc1cc(N2CCC(N(C)C3COC3)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-4-(4- (methyl(oxetan-3- yl)amino)piperidin-1-yl)-5- (1-methyl-1H-pyrazol-4- yl)phenyl)pyrimidin-4-amine | 633.3 [M + H]⁺ |
| 27 | 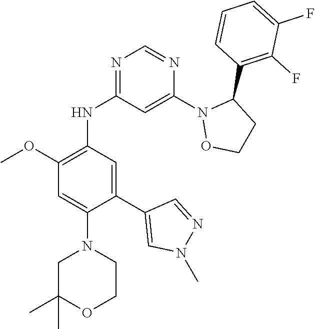 [SMILES: COc1cc(N2CCOC(C)(C)C2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(4-(2,2- dimethylmorpholino)-2- methoxy-5-(1-methyl-1H- pyrazol-4- yl)phenyl)pyrimidin-4-amine | 578.3 [M + H]⁺ |
| 28 | 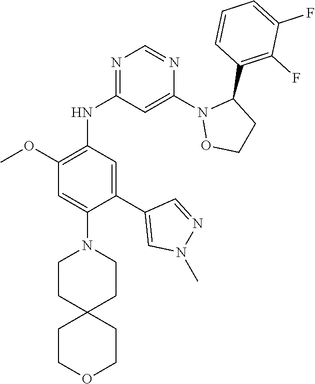 [SMILES: COc1cc(N2CCC3(CCOCC3)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- (3-oxa-9- azaspiro[5.5]undecan-9- yl)phenyl)pyrimidin-4-amine | ¹H NMR (400 MHz, Chloroform-d) δ 8.33 (d, J = 1.0 Hz, 1H), 7.97 (d, J = 0.7 Hz, 1H), 7.80 (s, 1H), 7.75 (s, 1H), 7.36-7.30 (m, 1H), 7.11- 7.02 (m, 2H), 6.94 (s, 1H), 6.72 (s, 1H), 6.51 (d, J = 1.1 Hz, 1H), 5.92 (dd, J = 8.8, 4.8 Hz, 1H), 4.12 (td, J = 7.9, 4.3 Hz, 1H), 3.96 (s, 3H), 3.93-3.86 (m, 4H), 3.69 (t, J = 5.4 Hz, 4H), 2.86 (t, J = 5.1 Hz, 4H), 2.84-2.76 (m, 1H), 2.37-2.26 (m, 1H), 1.65 (t, J = 5.6 Hz, 4H), 1.58 (t, J = 5.4 Hz, 4H).; 618.3 [M + H]⁺ |
| 29 | 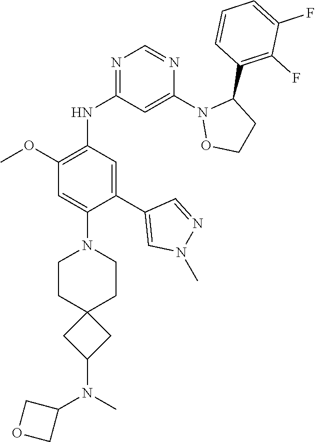 [SMILES: COc1cc(N2CCC3(CC2)CC(N(C)C2COC2)C3)c(-c2cnn(C)c2)cc1Nc1cc(N2OCC[C@@H]2c2cccc(F)c2F)ncn1] | (R)-7-(4-((6-(3-(2,3- difluorophenyl)isoxazolidin- 2-yl)pyrimidin-4-yl)amino)- 5-methoxy-2-(1-methyl-1H- pyrazol-4-yl)phenyl)-N- methyl-N-(oxetan-3-yl)-7- azaspiro[3.5]nonan-2-amine | 673.3 [M + H]⁺ |
| 30 | 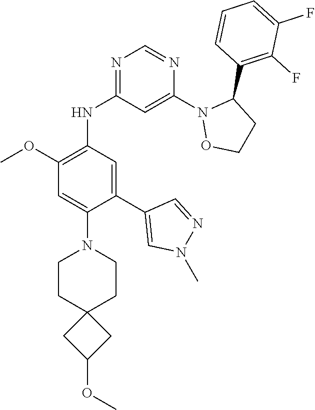 [SMILES: COc1cc(N2CCC3(CC2)CC(OC)C3)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-4-(2- methoxy-7- azaspiro[3.5]nonan-7-yl)-5- (1-methyl-1H-pyrazol-4- yl)phenyl)pyrimidin-4-amine | ¹H NMR (400 MHz, Chloroform-d) δ 8.33 (d, J = 1.0 Hz, 1H), 7.94-7.90 (m, 1H), 7.85 (s, 1H), 7.77 (s, 1H), 7.37-7.30 (m, 1H), 7.11-7.01 (m, 2H), 6.83 (s, 1H), 6.70 (s, 1H), 6.52 (d, J = 1.0 Hz, 1H), 5.92 (dd, J = 8.8, 4.7 Hz, 1H), 4.11 (td, J = 7.9, 4.1 Hz, 1H), 3.95 (s, 3H), 3.93-3.86 (m, 5H), 3.24 (s, 3H), 2.88-2.75 (m, 5H), 2.38- 2.28 (m, 1H), 2.28-2.19 (m, 2H), 1.76-1.71 (m, 2H), 1.71-1.67 (m, 2H), 1.64 (t, J = 5.3 Hz, 2H).; 618.3 [M + H]⁺ |
| 31 | 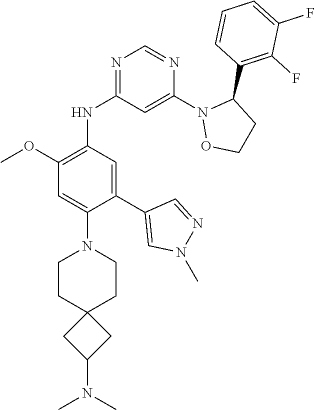 [SMILES: COc1cc(N2CCC3(CC2)CC(N(C)C)C3)c(-c2cnn(C)c2)cc1Nc1cc(N2OCC[C@@H]2c2cccc(F)c2F)ncn1] | (R)-7-(4-((6-(3-(2,3- difluorophenyl)isoxazolidin- 2-yl)pyrimidin-4-yl)amino)- 5-methoxy-2-(1-methyl-1H- pyrazol-4-yl)phenyl)-N,N- dimethyl-7- azaspiro[3.5]nonan-2-amine | ¹H NMR (400 MHz, DMSO) δ 8.63 (s, 1H), 8.16 (d, J = 1.0 Hz, 1H), 8.05 (s, 1H), 7.84 (s, 1H), 7.58 (s, 1H), 7.35 (dtd, J = 9.8, 7.9, 1.7 Hz, 1H), 7.28 (t, ) = 7.0 Hz, 1H), 7.23-7.16 (m, 1H), 6.78 (s, 1H), 6.28 (s, 1H), 5.76-5.71 (m, 1H), 4.17 (td, J = 7.9, 3.7 Hz, 1H), 3.86 (s, 3H), 3.86-3.81 (m, 1H), 3.81 (s, 3H), 2.85-2.79 (m, 1H), 2.74 (d, J = 20.6 Hz, 5H), 2.23-2.17 (m, 1H), 2.10 (s, 5H), 2.02 (d, J = 10.6 Hz, 3H), 1.63 (dt, J = 21.5, 5.5 Hz, 6H).; 631.3 [M + H]⁺ |
| 32 | 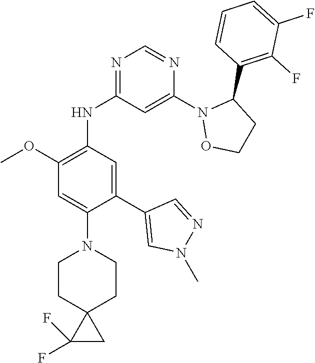 [SMILES: COc1cc(N2CCC3(CC2)CC3(F)F)c(-c2cnn(C)c2)cc1Nc1cc(N2OCC[C@@H]2c2cccc(F)c2F)ncn1] | (R)-N-(4-(1,1-difluoro-6- azaspiro[2.5]octan-6-yl)-2- methoxy-5-(1-methyl-1H- pyrazol-4-yl)phenyl)-6-(3- (2,3- difluorophenyl)isoxazolidin- 2-yl)pyrimidin-4-amine | ¹H NMR (400 MHz, Chloroform-d) δ 8.33 (d, J = 1.0 Hz, 1H), 7.93 (d, J = 0.7 Hz, 1H), 7.85 (s, 1H), 7.80 (s, 1H), 7.36-7.30 (m, 1H), 7.11- 7.02 (m, 2H), 6.98 (s, 1H), 6.72 (s, 1H), 6.52 (d, J = 1.1 Hz, 1H), 5.92 (dd, J = 8.8, 4.7 Hz, 1H), 4.12 (td, J = 7.9, 4.1 Hz, 1H), 3.97 (s, 3H), 3.95-3.87 (m, 4H), 3.02-2.92 (m, 2H), 2.91-2.76 (m, 3H), 2.37-2.27 (m, 1H), 1.80-1.68 (m, 6H).; 610.3 [M + H]⁺ |
| 33 | 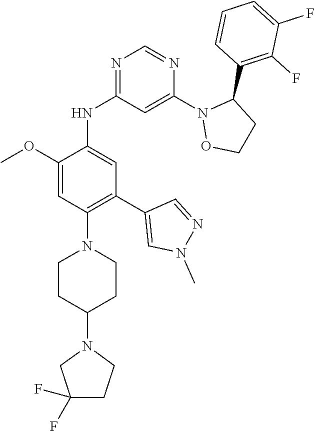 [SMILES: COc1cc(N2CCC(N3CCC(F)(F)C3)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(4-(4-(3,3- difluoropyrrolidin-1- yl)piperidin-1-yl)-2- methoxy-5-(1-methyl-1H- pyrazol-4- yl)phenyl)pyrimidin-4-amine | 653.3 [M + H]⁺ |
| 34 | 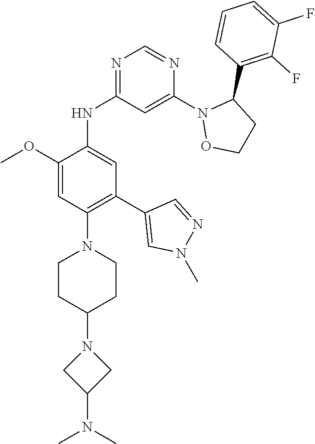 [SMILES: COc1cc(N2CCC(N3CC(N(C)C)C3)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(4-(4-(3- (dimethylamino)azetidin-1- yl)piperidin-1-yl)-2- methoxy-5-(1-methyl-1H- pyrazol-4- yl)phenyl)pyrimidin-4-amine | 646.3 [M + H]⁺ |
| 35 | 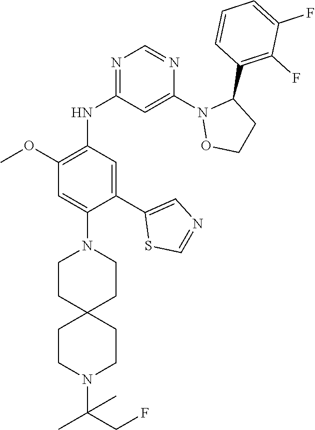 [SMILES: COc1cc(N2CCC3(CC2)CCN(C(C)(C)CF)CC3)c(-c2cncs2)cc1Nc1cc(N2OCC[C@@H]2c2cccc(F)c2F)ncn1] | (R)-6-(3-(2,3- difluorophenyl)isoxazolidin- 2-yl)-N-(4-(9-(1-fluoro-2- methylpropan-2-yl)-3,9- diazaspiro[5.5]undecan-3- yl)-2-methoxy-5-(thiazol-5- yl)phenyl)pyrimidin-4-amine | 694.3 [M + H]⁺ |
| 36 | 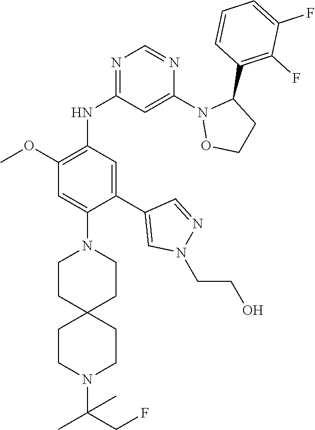 [SMILES: COc1cc(N2CCC3(CC2)CCN(C(C)(C)CF)CC3)c(-c2cnn(CCO)c2)cc1Nc1cc(N2OCC[C@@H]2c2cccc(F)c2F)ncn1] | (R)-2-(4-(5-((6-(3-(2,3- difluorophenyl)isoxazolidin- 2-yl)pyrimidin-4-yl)amino)- 2-(9-(1-fluoro-2- methylpropan-2-yl)-3,9- diazaspiro[5.5]undecan-3- yl)-4-methoxyphenyl)-1H- pyrazol-1-yl)ethan-1-ol | 721.3 [M + H]⁺ |
| 37 | 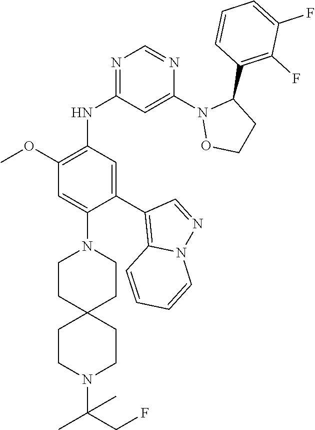 [SMILES: COc1cc(N2CCC3(CC2)CCN(C(C)(C)CF)CC3)c(-c2cnn3ccccc23)cc1Nc1cc(N2OCC[C@@H]2c2cccc(F)c2F)ncn1] | (R)-6-(3-(2,3- difluorophenyl)isoxazolidin- 2-yl)-N-(4-(9-(1-fluoro-2- methylpropan-2-yl)-3,9- diazaspiro[5.5]undecan-3- yl)-2-methoxy-5- (pyrazolo[1,5-a]pyridin-3- yl)phenyl)pyrimidin-4-amine | 727.3 [M + H]⁺ |
| 38 | 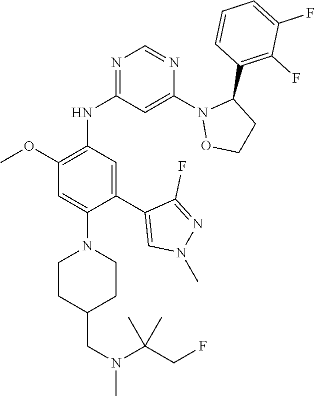 [SMILES: COc1cc(N2CCC(CN(C)C(C)(C)CF)CC2)c(-c2cn(C)nc2F)cc1Nc1cc(N2OCC[C@@H]2c2cccc(F)c2F)ncn1] | (R)-6-(3-(2,3- difluorophenyl)isoxazolidin- 2-yl)-N-(5-(3-fluoro-1- methyl-1H-pyrazol-4-yl)-4- (4-(((1-fluoro-2- methylpropan-2- yl)(methyl)amino)methyl)pi- peridin-1-yl)-2- methoxyphenyl)pyrimidin-4- amine | 683.3 [M + H]⁺ |
| 39 | 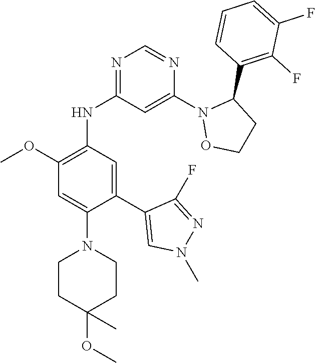 [SMILES: COc1cc(N2CCC(C)(OC)CC2)c(-c2cn(C)nc2F)cc1Nc1cc(N2OCC[C@@H]2c2cccc(F)c2F)ncn1] | (R)-6-(3-(2,3- difluorophenyl)isoxazolidin- 2-yl)-N-(5-(3-fluoro-1- methyl-1H-pyrazol-4-yl)-2- methoxy-4-(4-methoxy-4- methylpiperidin-1- yl)phenyl)pyrimidin-4-amine | 610.3 [M + H]⁺ |
| 40 | 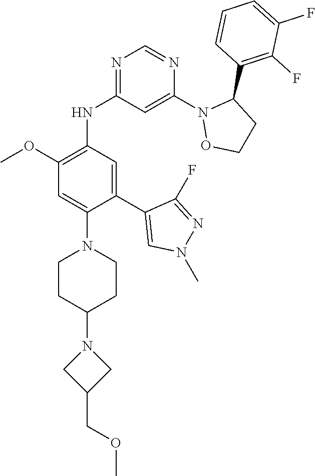 [SMILES: COCC1CN(C2CCN(c3cc(OC)c(Nc4cc(N5OCC[C@@H]5c5cccc(F)c5F)ncn4)cc3-c3cn(C)nc3F)CC2)C1] | (R)-6-(3-(2,3- difluorophenyl)isoxazolidin- 2-yl)-N-(5-(3-fluoro-1- methyl-1H-pyrazol-4-yl)-2- methoxy-4-(4-(3- (methoxymethyl)azetidin-1- yl)piperidin-1- yl)phenyl)pyrimidin-4-amine | 665.3 [M + H]⁺ |
| 41 | 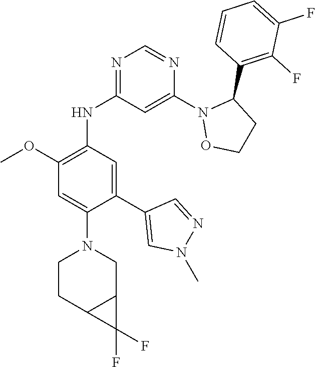 | N-(4-(7,7-difluoro-3- azabicyclo[4.1.0]heptan-3- yl)-2-methoxy-5-(1-methyl- 1H-pyrazol-4-yl)phenyl)-6- ((R)-3-(2,3- difluorophenyl)isoxazolidin- 2-yl)pyrimidin-4-amine | ¹H NMR (400 MHz, Chloroform-d) δ 8.34 (s, 1H), 7.87 (s, 1H), 7.85 (d, J = 2.7 Hz, 1H), 7.81 (d, J = 5.8 Hz, 1H), 7.36-7.30 (m, 1H), 7.12- 7.01 (m, 2H), 6.90 (s, 1H), 6.67 (s, 1H), 6.53 (dd, J = 4.4, 1.1 Hz, 1H), 5.92 (dd, J = 8.8, 4.7 Hz, 1H), 4.16-4.09 (m, 1H), 3.95 (s, 3H), 3.94- 3.89 (m, 1H), 3.89-3.87 (m, 3H), 3.21 (dt, J = 13.5, 7.2 Hz, 1H), 3.06 (d, J = 12.1 Hz, 1H), 2.84 (dtd, J = 12.3, 8.1, 4.2 Hz, 1H), 2.70 (t, J = 5.5 Hz, 2H), 2.37-2.26 (m, 1H), 2.05-1.96 (m, 1H), 1.92-1.85 (m, 1H), 1.83-1.73 (m, 2H).; 569.3 [M + H]⁺ |
| 42 | 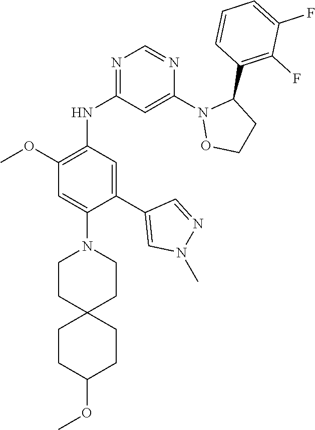 [SMILES: COc1cc(N2CCC3(CCC(OC)CC3)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-4-(9- methoxy-3- azaspiro[5.5]undecan-3-yl)- 5-(1-methyl-1H-pyrazol-4- yl)phenyl)pyrimidin-4-amine | ¹H NMR (400 MHz, Chloroform-d) δ 8.32 (s, 1H), 7.94 (s, 1H), 7.83 (s, 1H), 7.75 (s, 1H), 7.35-7.30 (m, 1H), 7.08-7.02 (m, 2H), 6.80 (s, 1H), 6.74 (s, 1H), 6.52 (s, 1H), 5.94-5.89 (m, 1H), 4.14-4.07 (m, 1H), 3.94 (s, 3H), 3.92-3.86 (m, 4H), 3.35 (s, 3H), 3.23-3.16 (m, 1H), 2.86- 2.79 (m, 5H), 2.35-2.27 (m, 1H), 1.80-1.72 (m, 4H), 1.62-1.59 (m, 4H), 1.51-1.44 (m, 4H).; 646.3 [M + H]⁺ |
| 43 | 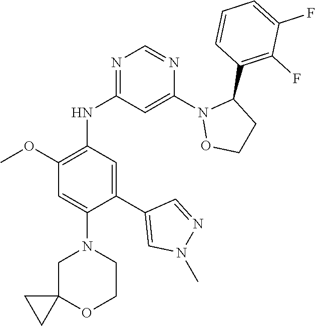 [SMILES: COc1cc(N2CCOC3(CC3)C2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- (4-oxa-7-azaspiro[2.5]octan- 7-yl)phenyl)pyrimidin-4- amine | ¹H NMR (400 MHz, MeOD-d4, TFA form) δ 8.14 (s, 1H), 8.06 (s, 1H), 7.94 (s, 1H), 7.32 (s, 1H), 7.28-7.13 (m, 3H), 6.92 (s, 1H), 6.01 (s, 1H), 5.77 (t, J = 7.3 Hz, 1H), 4.48-4.39 (m, 1H), 4.23-4.13 (m, 1H), 3.95 (s, 3H), 3.91-3.84 (m, 5H), 3.13-3.00 (m, 3H), 2.91 (s, 2H), 2.52- 2.39 (m, 1H), 0.91-0.75 (m, 2H), 0.56-0.48 (m, 2H).); 576.3. [M + H]⁺ |
| 44 | 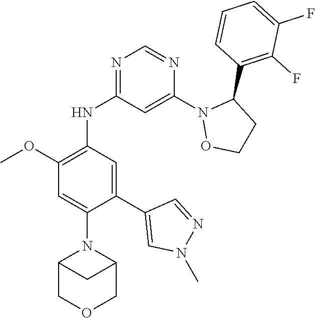 [SMILES: COc1cc(N2C3COCC2C3)c(-c2cnn(C)c2)cc1Nc1cc(N2OCC[C@@H]2c2cccc(F)c2F)ncn1] | N-(4-(3-oxa-6- azabicyclo[3.1.1]heptan-6- yl)-2-methoxy-5-(1-methyl- 1H-pyrazol-4-yl)phenyl)-6- ((R)-3-(2,3- difluorophenyl)isoxazolidin- 2-yl)pyrimidin-4-amine | ¹H NMR (400 MHz, Methanol-d₄) δ 8.07 (s, 1H), 7.61 (s, 1H), 7.49 (s, 1H), 7.30 (t, J = 6.9 Hz, 1H), 7.18 (s, 1H), 7.16-7.09 (m, 2H), 6.38 (s, 1H), 6.28 (s, 1H), 5.75 (dd, J = 8.8, 4.8 Hz, 1H), 4.16 (dd, J = 10.7, 3.3 Hz, 2H), 4.11 (dd, J = 7.9, 4.2 Hz, 1H), 3.98 (d, J = 6.1 Hz, 2H), 3.94-3.89 (m, 2H), 3.88 (s, 3H), 3.83 (s, 3H), 3.54 (d, J = 10.9 Hz, 2H), 2.87-2.81 (m, 1H), 2.64 (q, J = 6.8 Hz, 1H), 2.27-2.21 (m, 1H), 1.82 (d, J = 8.1 Hz, 1H); 562.3 [M + H]⁺ |
| 45 | 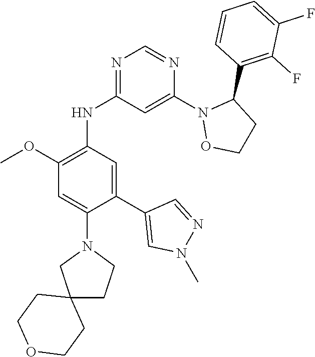 [SMILES: COc1cc(N2CCC3(CCOCC3)C2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- (8-oxa-2- azaspiro[4.5]decan-2- yl)phenyl)pyrimidin-4-amine | ¹H NMR (400 MHz, MeOD-d4, TFA form) δ 8.11 (s, 1H), 7.70 (s, 1H), 7.59 (s, 1H), 7.30- 7.14 (m, 4H), 7.11 (s, 1H), 6.69 (s; 1H), 6.01 (s, 1H), 5.77 (s, 1H), 4.47-4.38 (m, 1H), 4.22-4.12 (m, 1H), 3.94 (s, 3H), 3.87 (s, 3H), 3.78-3.57 (m, 4H), 3.18-3.10 (m, 2H), 3.10-3.03 (m, 1H), 3.00 (s, 2H), 2.51-2.38 (m, 1H), 1.83 (t, J = 7.0 Hz, 2H), 1.63 (t, J = 5.5 Hz, 4H); 604.3 [M + H]⁺ |
| 46 | 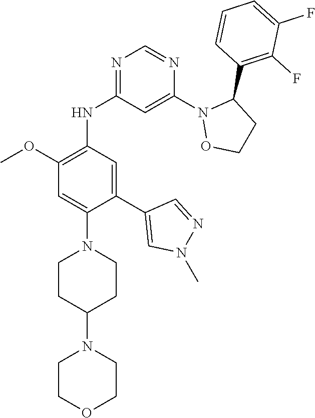 [SMILES: COc1cc(N2CCC(N3CCOCC3)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- (4-morpholinopiperidin-1- yl)phenyl)pyrimidin-4-amine | ¹H NMR (400 MHz, DMSO-d₆) δ 8.61 (s, 1H), 8.16 (s, 1H), 8.04 (s, 1H), 7.84 (s, 1H), 7.36- 7.26 (m, 2H), 7.21-7.18 (m, 1H), 6.79 (s, 1H), 6.30 (s, 1H), 5.77-5.73 (m, J = 8.8, 4.8 Hz, 1H), 4.19-4.14 (m, J = 7.8, 4.0 Hz, 1H), 3.86 (s, 3H), 3.81 (s, 3H), 3.65-3.54 (m, 6H), 3.15-3.09 (m, J = 11.4 Hz, 2H), 2.83-2.77 (m, 1H), 2.66-2.54 (m, J = 11.3 Hz, 3H), 2.27-2.12 (m, J = 13.5, 7.2 Hz, 3H), 1.91-1.79 (m, J = 11.1 Hz, 3H), 1.58-1.47 (m, J = 21.0, 11.8 Hz, 3H).; 633.3 [M + H]⁺ |
| 47 | 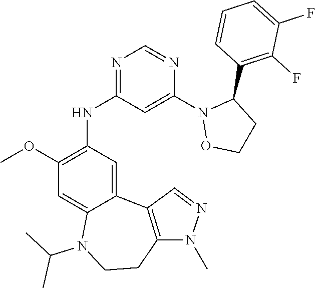 [SMILES: COc1cc2c(cc1Nc1cc(N3OCC[C@@H]3c3cccc(F)c3F)ncn1)-c1cnn(C)c1CCN2C(C)C] | (R)-N-(6-(3-(2,3- difluorophenyl)isoxazolidin- 2-yl)pyrimidin-4-yl)-6- isopropyl-8-methoxy-3- methyl-3,4,5,6- tetrahydrobenzo[b]pyrazolo [4,3-d]azepin-9-amine | ¹H NMR (400 MHz, DMSO-d₆) δ 8.53-8.50 (m, 1H), 8.15 (s, 1H), 7.73 (s, 1H), 7.60 (s, 1H), 7.36-7.26 (m, 2H), 7.22-7.17 (m, J = 7.8, 5.1 Hz, 1H), 6.58 (s, 1H), 5.78-5.74 (m, J = 8.7, 5.0 Hz, 1H), 4.17-4.10 (m, J = 7.8, 3.7 Hz, 2H), 3.96-3.92 (m, 2H), 3.78 (s, 3H), 3.74 (s, 3H), 3.23-3.16 (m, J = 11.0, 5.1 Hz, 2H), 2.94-2.90( m, J = 5.1 Hz, 2H), 2.83-2.77 (m, J = 8.1 Hz, 1H), 2.23-2.16 (m, 1H), 1.27 (d, J = 6.4 Hz, 6H).; 548.3 [M + H]⁺ |
| 48 | 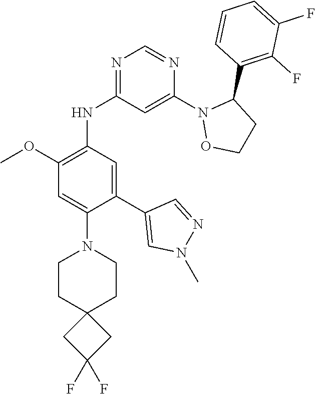 [SMILES: COc1cc(N2CCC3(CC2)CC(F)(F)C3)c(-c2cnn(C)c2)cc1Nc1cc(N2OCC[C@@H]2c2cccc(F)c2F)ncn1] | (R)-N-(4-(2,2-difluoro-7- azaspiro[3.5]nonan-7-yl)-2- methoxy-5-(1-methyl-1H- pyrazol-4-yl)phenyl)-6-(3- (2,3- difluorophenyl)isoxazolidin- 2-yl)pyrimidin-4-amine | ¹H NMR (400 MHz, Chloroform-d) δ 8.33 (s, 1H), 7.92 (s, 1H), 7.81 (s, 1H), 7.79 (s, 1H), 7.35-7.30 (m, 1H), 7.08-7.02 (m, 2H), 6.83 (s, 1H), 6.68 (s, 1H), 6.52 (s, 1H), 5.94-5.89 (m, 1H), 4.14-4.08 (m, 1H), 3.95 (s, 1H), 3.92-3.86 (m, 4H), 2.87-2.77 (m, 5H), 2.39 (t, J = 12.6 Hz, 4H), 2.33-2.26 (m, 1H), 1.78-1.73 (m, 4H).; 624.3 [M + H]⁺ |
| 49 | 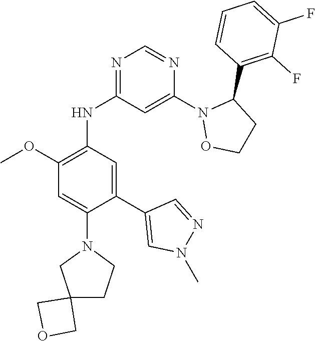 [SMILES: COc1cc(N2CCC3(COC3)C2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- (2-oxa-6-azaspiro[3.4]octan- 6-yl)phenyl)pyrimidin-4- amine | 576.3 [M + H]⁺ |
| 50 | 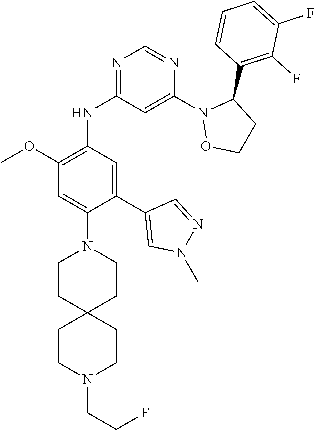 [SMILES: COc1cc(N2CCC3(CCN(CCF)CC3)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(4-(9-(2- fluoroethyl)-3,9- diazaspiro[5.5]undecan-3- yl)-2-methoxy-5-(1-methyl- 1H-pyrazol-4- yl)phenyl)pyrimidin-4-amine | ¹H NMR (400 MHz, Chloroform-d) δ 8.32 (d, J = 1.0 Hz, 1H), 7.95 (s, 1H), 7.81 (s, 1H), 7.73 (s, 1H), 7.36-7.30 (m, 1H), 7.11-7.02 (m, 2H), 6.98 (s, 1H), 6.73 (s, 1H), 6.50 (d, J = 1.0 Hz, 1H), 5.91 (dd, J = 8.8, 4.7 Hz, 1H), 4.66 (t, J = 4.9 Hz, 1H), 4.54 (t, J = 4.9 Hz, 1H), 4.11 (td, J = 7.9, 4.2 Hz, 1H), 3.95 (s, 3H), 3.94-3.86 (m, 4H), 2.89-2.80 (m, 5H), 2.78 (t, J = 5.0 Hz, 1H), 2.71 (t, J = 4.9 Hz, 1H), 2.59-2.50 (m, 4H), 2.36-2.26 (m, 1H), 1.61 (dt, J = 18.8, 5.7 Hz, 8H).; 663.3 [M + H]⁺ |
| 51 | 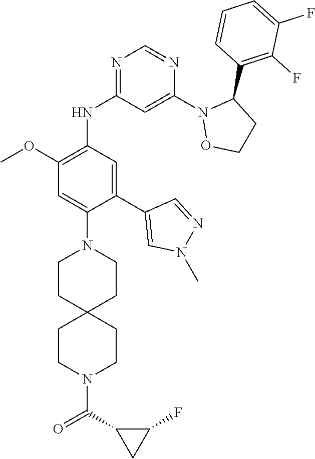 [SMILES: COc1cc(N2CCC3(CCN(C(=O)[C@H]4C[C@H]4F)CC3)CC2)c(-c2cnn(C)c2)cc1Nc1cc(N2OCC[C@@H]2c2cccc(F)c2F)ncn1] | (9-(4-((6-((R)-3-(2,3- difluorophenyl)isoxazolidin- 2-yl)pyrimidin-4-yl)amino)- 5-methoxy-2-(1-methyl-1H- pyrazol-4-yl)phenyl)-3,9- diazaspiro[5.5]undecan-3- yl)((1 R,2R)-2- fluorocyclopropyl)methanone | 703.3 [M + H]⁺ |
| 52 | 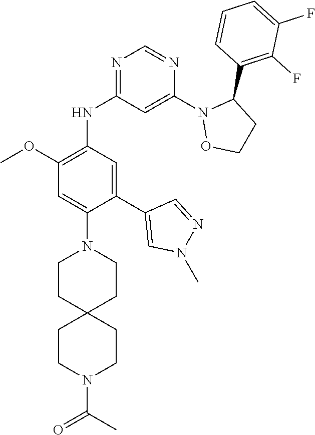 [SMILES: COc1cc(N2CCC3(CCN(C(C)=O)CC3)CC2)c(-c2cnn(C)c2)cc1Nc1cc(N2OCC[C@@H]2c2cccc(F)c2F)ncn1] | (R)-1-(9-(4-((6-(3-(2,3- difluorophenyl)isoxazolidin- 2-yl)pyrimidin-4-yl)amino)- 5-methoxy-2-(1-methyl-1H- pyrazol-4-yl)phenyl)-3,9- diazaspiro[5.5]undecan-3- yl)ethan-1-one | 659.3 [M + H]⁺ |
| 53 | 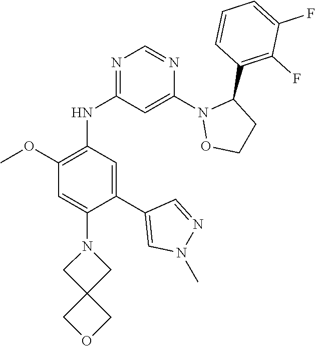 [SMILES: COc1cc(N2CC3(COC3)C2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- (2-oxa-6- azaspiro[3.3]heptan-6- yl)phenyl)pyrimidin-4-amine | 562.3 [M + H]⁺ |
| 54 | 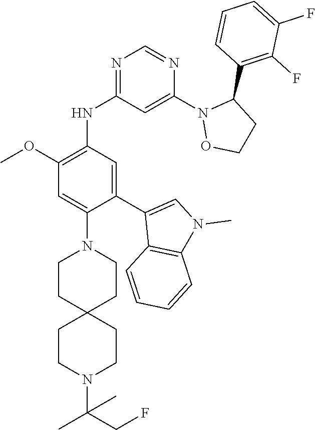 [SMILES: COc1cc(N2CCC3(CC2)CCN(C(C)(C)CF)CC3)c(-c2cn(C)c3ccccc23)cc1Nc1cc(N2OCC[C@@H]2c2cccc(F)c2F)ncn1] | (R)-6-(3-(2,3- difluorophenyl)isoxazolidin- 2-yl)-N-(4-(9-(1-fluoro-2- methylpropan-2-yl)-3,9- diazaspiro[5.5]undecan-3- yl)-2-methoxy-5-(1-methyl- 1H-indol-3- yl)phenyl)pyrimidin-4-amine | 740.3 [M + H]⁺ |
| 55 | 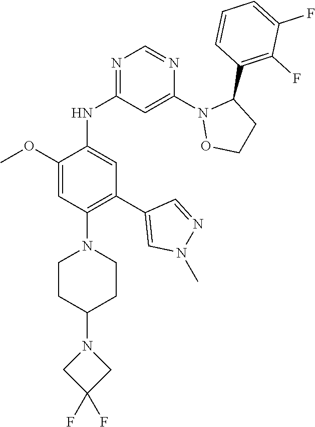 [SMILES: COc1cc(N2CCC(N3CC(F)(F)C3)CC2)c(-c2cnn(C)c2)cc1Nc1cc(N2OCC[C@@H]2c2cccc(F)c2F)ncn1] | (R)-N-(4-(4-(3,3- difluoroazetidin-1- yl)piperidin-1-yl)-2- methoxy-5-(1-methyl-1H- pyrazol-4-yl)phenyl)-6-(3- (2,3- difluorophenyl)isoxazolidin- 2-yl)pyrimidin-4-amine | ¹H NMR (400 MHz, CDCl₃) δ 8.30 (d, J = 0.9 Hz, 1H), 7.88 (s, 1H), 7.84 (d, J = 6.9 Hz, 1H), 7.73 (s, 1H), 7.31 (t, J = 6.8 Hz, 1H), 7.21 (s, 1H), 7.07 (ddd, J = 16.6, 7.9, 4.7 Hz, 2H), 6.70 (s, 1H), 6.48 (d, ) = 0.8 Hz, 1H), 5.91 (dd, J = 8.7, 4.8 Hz, 1H), 4.15-4.09 (m, 1H), 3.94 (s, 3H), 3.93-3.89 (m, 1H), 3.88 (s, 3H), 3.60 (t, J = 11.8 Hz, 4H), 3.17 (d, J = 11.9 Hz, 2H), 2.90-2.78 (m, 1H), 2.61 (td, J = 10.8, 2.4 Hz, 2H), 2.37-2.21 (m, 2H), 1.77 (d, J = 12.3. Hz, 2H), 1.57-1.44 (m, 2H); 639.4 [M + H]⁺ |
| 56 | 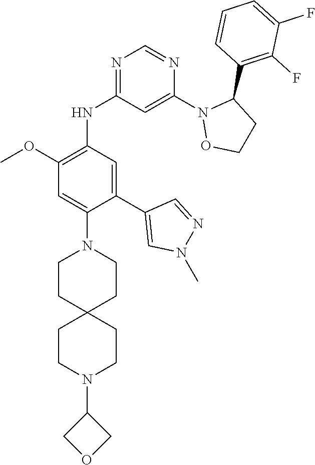 [SMILES: COc1cc(N2CCC3(CC2)CCN(C2COC2)CC3)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- (9-(oxetan-3-yl)-3,9- diazaspiro[5.5]undecan-3- yl)phenyl)pyrimidin-4-amine | ¹H NMR (400 MHz, DMSO) δ 8.60 (s, 1H), 8.15 (s, 1H), 8.05 (s, 1H), 7.85 (s, 1H), 7.57 (s, 1H), 7.40-7.25 (m, 2H), 7.22-7.16 (m, 1H), 6.84 (s, 1H), 6.28 (s, 1H), 5.78-5.72 (m, 1H), 4.51 (t, J = 6.4 Hz, 2H), 4.41 (t, J = 6.0 Hz, 2H), 4.19- 4.13 (m, 1H), 3.88-3.82 (m, 4H), 3.81 (s, 3H), 3.41-3.37 (m, 1H), 2.85-2.76 (m, 5H); 2.25- 2.14 (m, 5H), 1.53 (s, 8H).; 673.5 [M + H]⁺ |
| 57 | 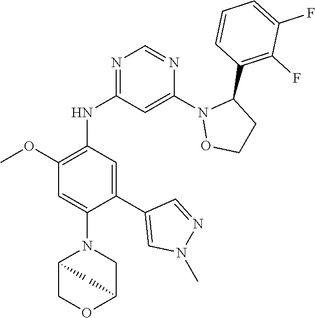 [SMILES: COc1cc(N2C[C@@H]3C[C@H]2CO3)c(-c2cnn(C)c2)cc1Nc1cc(N2OCC[C@@H]2c2cccc(F)c2F)ncn1] | N-(4-((15,4S)-2-oxa-5- azabicyclo[2.2.1]heptan-5- yl)-2-methoxy-5-(1-methyl- 1H-pyrazol-4-yl)phenyl)-6- ((R)-3-(2,3- difluorophenyl)isoxazolidin- 2-yl)pyrimidin-4-amine | 562.3 [M + H]⁺ |
| 58 | 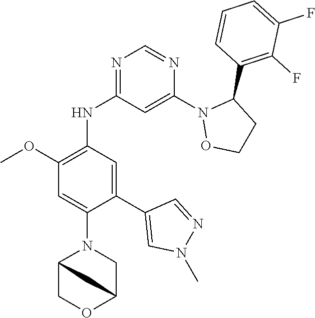 [SMILES: COc1cc(N2C[C@H]3C[C@@H]2CO3)c(-c2cnn(C)c2)cc1Nc1cc(N2OCC[C@@H]2c2cccc(F)c2F)ncn1] | N-(4-((1 R,4R)-2-oxa-5- azabicyclo [2.2.1]heptan-5- yl)-2-methoxy-5-(1-methyl- 1H-pyrazol-4-yl)phenyl)-6- (R)-3-(2,3- difluorophenyl)isoxazolidin- 2-yl)pyrimidin-4-amine | 562.3 [M + H]⁺ |
| 59 | 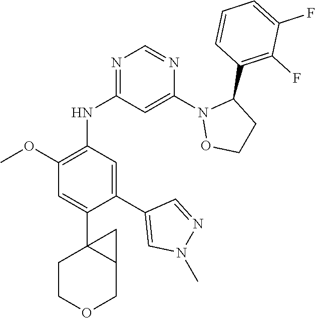 [SMILES: COc1cc(C23CCOCC2C3)c(-c2cnn(C)c2)cc1Nc1cc(N2OCC[C@@H]2c2cccc(F)c2F)ncn1] | N-(4-(3- oxabicyclo[4.1.0]heptan-6- yl)-2-methoxy-5-(1-methyl- 1H-pyrazol-4-yl)phenyl)-6- ((R)-3-(2,3- difluorophenyl)isoxazolidin- 2-yl)pyrimidin-4-amine | ¹H NMR (400 MHz, Chloroform-d) δ 8.34 (s, 1H), 7.85 (s, 1H), 7.60 (s, 1H), 7.56 (d, J = 4.3 Hz, 1H), 7.35-7.29 (m, 1H), 7.09-7.02 (m, 2H), 7.00 (s, 1H), 6.93 (s, 1H), 6.59 (s, 1H), 5.94- 5.88 (m, 1H), 4.16-4.10 (m, 1H), 3.99 (s, 3H), 3.94-3.90 (m, 4H), 3.89-3.86 (m, 2H), 3.51- 3.44 (m, 1H), 3.25-3.16 (m, 1H), 2.88-2.78 (m, 1H), 2.36-2.27 (m, 1H), 2.08-2.00 (m, 1H), 1.90-1.83 (m, 1), 1.19-1.13 (m, 1H), 0.99-0.94 (m, 1H), 0.86-0.82 (m, 1H).; 561.3 [M + H]⁺ |
| 60 | 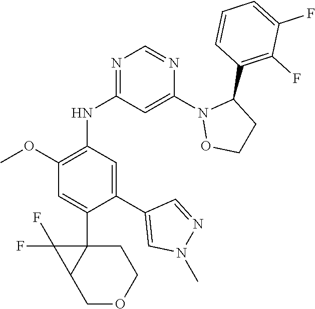 [SMILES: COc1cc(C23CCOCC2C3(F)F)c(-c2cnn(C)c2)cc1Nc1cc(N2OCC[C@@H]2c2cccc(F)c2F)ncn1] | N-(4-(7,7-difluoro-3- oxabicyclo[4.1.0]heptan-6- yl)-2-methoxy-5-(1-methyl- 1H-pyrazol-4-yl)phenyl)-6- ((R)-3-(2,3- difluorophenyl)isoxazolidin- 2-yl)pyrimidin-4-amine | ¹H NMR (400 MHz, DMSO) δ 8.76 (d, J = 3.9 Hz, 1H), 8.21 (s, 1H), 7.86 (d, J = 5.5 Hz, 1H), 7.74 (s, 1H), 7.48 (s, 1H), 7.40-7.25 (m, 2H), 7.24-7.16 (m, 1H), 7.02 (s, 1H), 6.59 (s, 1H), 5.78-5.71 (m, 1H), 4.25-4.17 (m, 1H), 3.91- 3.84 (m, 7H), 3.46-3.33 (m, 5H), 2.89-2.75 (m, 1H), 2.28-2.18 (m, 1H), 1.90 (s, 1H), 1.60 (s, 1H). 597.4 [M + H]⁺ |
| 61 | 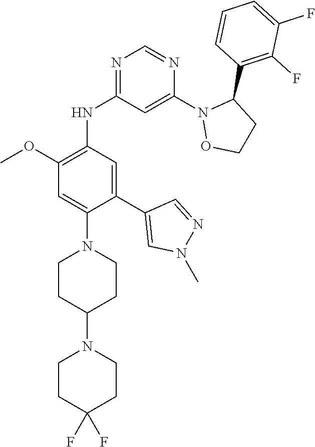 [SMILES: COc1cc(N2CCC(N3CCC(F)(F)CC3)CC2)c(-c2cnn(C)c2)cc1Nc1cc(N2OCC[C@@H]2c2cccc(F)c2F)ncn1] | (R)-N-(4-(4,4-difluoro-[1,4′- bipiperidin]-1′-yl)-2- methoxy-5-(1-methyl-1H- pyrazol-4-yl)phenyl)-6-(3- (2,3- difluorophenyl)isoxazolidin- 2-yl)pyrimidin-4-amine | ¹H NMR (400 MHz, CDCl₃) δ 8.32 (s, 1H), 7.94 (d, J = 8.6 Hz, 1H), 7.79 (t, J = 12.7 Hz, 2H), 7.31 (dd, J = 14.6, 9.0 Hz, 1H), 7.11-6.98 (m, 3H), 6.71 (d, J = 8.5 Hz, 1H), 6.50 (s, 1H), 5.92 (dd, J = 8.5, 4.7 Hz, 1H), 4.11 (td, J = 7.8, 4.1 Hz, 1H), 3.96 (d, J = 8.5 Hz, 3H), 3.92-3.84 (m, 4H), 3.24 (d, J = 11.4 Hz, 2H), 2.88-2.79 (m, 1H), 2.77-2.69 (m, 4H), 2.59 (dd, J = 19.7, 9.8 Hz, 2H), 2.44 (t, J = 11.4 Hz, 1H), 2.32 (dt, J = 8.2, 6.1 Hz, 1H), 2.02 (dd, J = 12.5, 6.7 Hz, 4H), 1.87 (d, J = 10.3 Hz, 2H), 1.70-1.60 (m, 2H); 667.4 [M + H]⁺ |
| 62 | 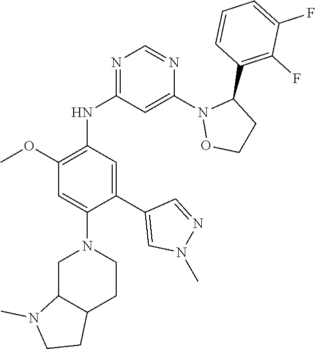 [SMILES: COc1cc(N2CCC3CCN(C)C3C2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N-(2-methoxy-5-(1- methyl-1H-pyrazol-4-yl)-4- (1-methyloctahydro-6H- pyrrolo[2,3-c]pyridin-6- yl)phenyl)pyrimidin-4-amine | ¹H NMR (400 MHz, DMSO) δ 8.62 (d, J = 1.9 Hz, 1H), 8.16 (d, J = 1.0 Hz, 1H), 8.09 (s, 1H), 7.85 (s, 1H), 7.59 (s, 1H), 7.39-7.31 (m, 1H), 7.29 (t, J = 7.1 Hz, 1H), 7.23-7.16 (m, 1H), 6.78 (s, 1H), 6.29 (s, 1H), 5.76-5.72 (m, 1H), 4.17 (td, J = 7.8, 3.6 Hz, 1H), 3.86 (s, 3H), 3.86-3.81 (m, 1H), 3.81 (s, 3H), 3.07 (s, 1H), 2.86-2.69 (m, 5H), 2.25-2.15 (m, 4H), 1.82 (d, J =3 2.0 Hz, 3H), 1.29-1.13 (m, 1H), 1.23 (s, 3H).; 603.3 [M + H]⁺ |
| 63 | 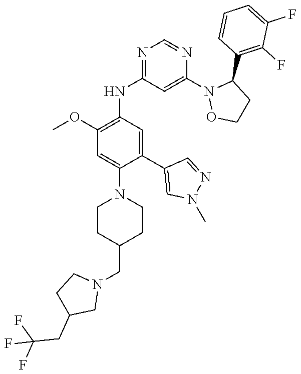 [SMILES: COc1cc(N2CCC(CN3CCC(CC(F)(F)F)C3)CC2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N-(2-methoxy-5-(1- methyl-1H-pyrazol-4-yl)-4- (4-((3-(2,2,2- trifluoroethyl)pyrrolidin-1- yl)methyl)piperidin-1- yl)phenyl)pyrimidin-4-amine | ¹H NMR (400 MHz, Methanol-d₄) δ 8.11 (s, 1H), 7.98 (s, 1H), 7.54 (s, 1H), 7.30 (t, J = 6.9 Hz, 1H), 7.17-7.08 (m, 2H), 6.83 (s, 1H), 6.33 (s, 1H), 5.76 (dd, J = 8.8, 4.9 Hz, 1H), 4.14- 4.10 (m, 1H), 3.90 (s, 3H), 3.85 (s, 3H), 3.16 (d, J = 11.6 Hz, 2H), 2.97 (t, J = 8.7 Hz, 1H), 2.89-2.79 (m, 2H), 2.65-2.57 (m, 3H), 2.51- 2.42 (m, 3H), 2.31-2.23 (m, 4H), 2.14-2.09 (m, 1H), 1.82 (d, J = 12.5 Hz, 2H), 1.68-1.52 (m, 3H), 1.35 (qd, J = 12.3, 11.6, 3.6 Hz, 3H). 713.4 [M + H]⁺ |
| 64 | 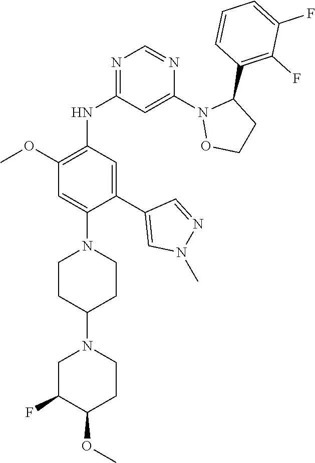 [SMILES: COc1cc(N2CCC(N3CC[C@@H](OC)[C@@H](F)C3)CC2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)- N-(4-((3,4R)-3-fluoro- 4-methoxy-[1,4′- bipiperidin]-1′-yl)-2- methoxy-5-(1-methyl-1H- pyrazol-4- yl)phenyl)pyrimidin-4-amine | ¹H NMR (400 MHz, Methanol-d₄) δ 8.11 (s, 1H), 7.99 (s, 1H), 7.55 (s, 1H), 7.29 (t, J = 6.0 Hz, 1H), 7.15-7.09 (m, 2H), 6.81 (s, 1H), 6.33 (s, 1H), 5.76 (dd, J = 8.5, 5.3 Hz, 1H), 4.14- 4.10 (m, 1H), 3.91 (s, 3H), 3.85 (s, 3H), 3.47- 3.45 (m, 1H), 3.41 (s, 3H), 3.21 (d, J = 11.1 Hz, 2H), 2.87-2.84 (m, 1H), 2.63 (t, J = 11.5 Hz, 4H), 2.46 (s, 4H), 2.26-2.22 (m, 1H), 1.91 (s, 4H), 1.82 (s, 2H), 1.69-1.63 (m, 2H); 679.5 [M + H]⁺ |
| 65 | 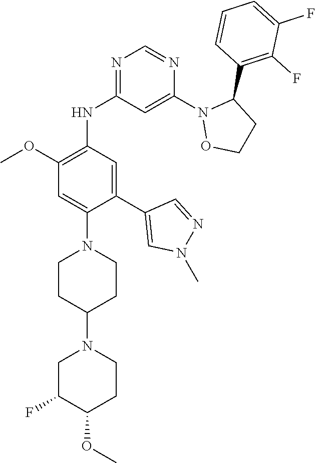 [SMILES: COc1cc(N2CCC(N3CC[C@H](OC)[C@H](F)C3)CC2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N-(4-((3R,45)-3-fluoro- 4-methoxy-[1,4′- bipiperidin]-1′-yl)-2- methoxy-5-(1-methyl-1H- pyrazol-4- yl)phenyl)pyrimidin-4-amine | 679.3 [M + H]⁺ |
| 66 | 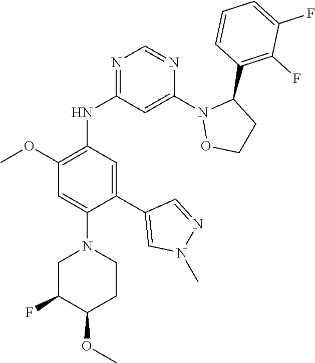 [SMILES: COc1cc(N2CC[C@@H](OC)[C@@H](F)C2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)- N-(4-((3,4R)-3-fluoro- 4-methoxypiperidin-1-yl)-2- methoxy-5-(1-methyl-1H- pyrazol-4- yl)phenyl)pyrimidin-4-amine | ¹H NMR (400 MHz, Methanol-d₄) δ 8.04 (s, 1H), 8.00 (s, 1H), 7.79 (s, 1H), 7.54 (s, 1H), 7.24-7.19 (m, 1H), 7.09-6.99 (m, 2H), 6.77 (s, 1H), 6.27 (s, 1H), 5.71-5.65 (m, 1H), 4.07-4.02 (m, 1H), 3.85-3.77 (m, 8H), 3.35 (s, 3H), 2.97- 2.93 (m, 1H), 2.80-2.74 (m, 1H), 2.65-2.60 (m, 1H), 2.22-2.12 (m, 2H), 1.90-1.80 (m, 2H), 1.77-1.69 (m, 2H); 596.3 [M + H]⁺ |
| 67 | 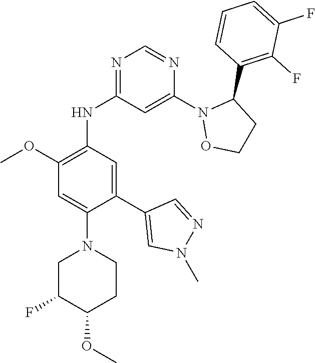 [SMILES: COc1cc(N2CC[C@H](OC)[C@H](F)C2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)- N-(4-((3R,4S)-3-fluoro- 4-methoxypiperidin-1-yl)-2- methoxy-5-(1-methyl-1H- pyrazol-4- yl)phenyl)pyrimidin-4-amine | ¹H NMR (400 MHz, DMSO) δ 8.66 (s, 1H), 8.17 (d, J = 1.0 Hz, 1H), 8.08 (s, 1H), 7.84 (d, J = 0.7 Hz, 1H), 7.65 (s, 1H), 7.40-7.26 (m, 2H), 7.24-7.16 (m, 1H), 6.83 (s, 1H), 6.33 (s, 1H), 5.78-5.71 (m, 1H), 5.04 (s, 1H), 4.92 (d, J = 5.0 Hz, 1H), 4.17 (td, J = 7.9, 3.7 Hz, 1H), 3.85 (s, 8H), 3.50 (t, J = 12.0 Hz, 1H), 3.27 (dt, J = 12.4, 6.9 Hz, 2H), 3.02 (dd, J = 27.0, 12.0 Hz, 1H), 2.90 (d, J = 10.6 Hz, 1H), 2.86-2.76 (m, 1H), 2.65 (t, J = 9.6 Hz, 1H), 2.26-2.15 (m, 1H), 1.88-1.72 (m, 2H).; 596.3 [M + H]⁺ |
| 68 | 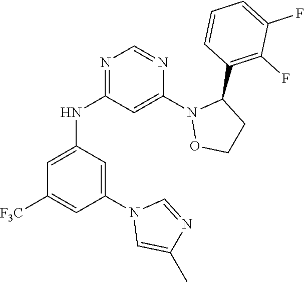 [SMILES: Cc1cn(-c2cc(Nc3cc(N4OCC[C@@H]4c4cccc(F)c4F)ncn3)cc(C(F)(F)F)c2)cn1] | (R)-6-(3-(2,3- difluorophenyl)isoxazolidin- 2-yl)-N-(3-(4-methyl-1H- imidazol-1-yl)-5- (trifluoromethyl)phenyl)pyri- midin-4-amine | 503.3 [M + H]⁺ |
| 69 | 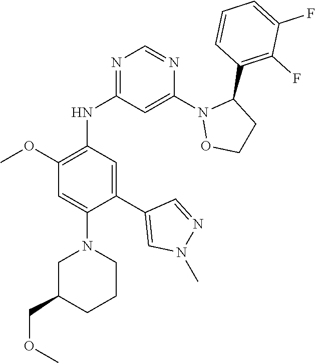 [SMILES: COC[C@@H]1CCCN(c2cc(OC)c(Nc3cc(N4OCC[C@@H]4c4cccc(F)c4F)ncn3)cc2-c2cnn(C)c2)C1] | 6-((R)-3-(2,3- difluorophenyl)isoxazolidin- 2-yl)-N-(2-methoxy-4-((R)- 3- (methoxymethyl)piperidin- 1-yl)-5-(1-methyl-1H- pyrazol-4- yl)phenyl)pyrimidin-4-amine | 592.3 [M + H]⁺ |
| 70 | 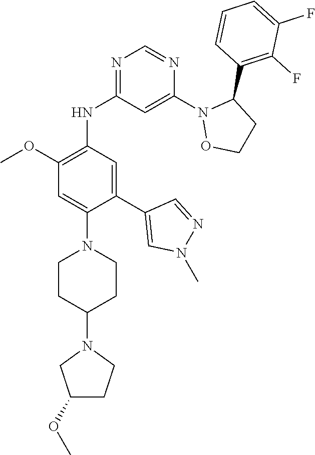 [SMILES: COc1cc(N2CCC(N3CC[C@H](OC)C3)CC2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N-(2-methoxy-4-(4- ((S)-3-methoxypyrrolidin-1- yl)piperidin-1-yl)-5-(1- methyl-1H-pyrazol-4- yl)phenyl)pyrimidin-4-amine | 647.4 [M + H]⁺ |
| 71 | 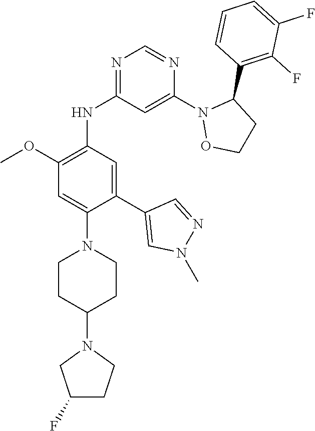 [SMILES: COc1cc(N2CCC(N3CC[C@H](F)C3)CC2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N-(4-(4-((S)-3- fluoropyrrolidin-1- yl)piperidin-1-yl)-2- methoxy-5-(1-methyl-1H- pyrazol-4- yl)phenyl)pyrimidin-4-amine | 635.4 [M + H]⁺ |
| 72 | 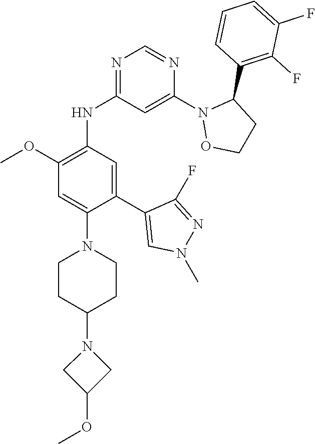 [SMILES: COc1cc(N2CCC(N3CC(OC)C3)CC2)c(-c2cn(C)nc2F)cc1Nc1cc(N2OCC[C@@H]2c2cccc(F)c2F)ncn1] | (R)-6-(3-(2,3- difluorophenyl)isoxazolidin- 2-yl)-N-(5-(3-fluoro-1- methyl-1H-pyrazol-4-yl)-2- methoxy-4-(4-(3- methoxyazetidin-1- yl)piperidin-1- yl)phenyl)pyrimidin-4-amine | 651.3 [M + H]⁺ |
| 73 | 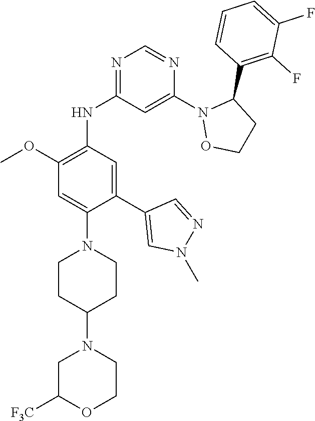 [SMILES: COc1cc(N2CCC(N3CCOC(C(F)(F)F)C3)CC2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N-(2-methoxy-5-(1- methyl-1H-pyrazol-4-yl)-4- (4-(2- (trifluoromethyl)morpholino) piperidin-1- yl)phenyl)pyrimidin-4-amine | ¹H NMR (400 MHz, MeOD-d4, TFA form) δ 8.15 (s, 1H), 7.99 (d, J = 6.8 Hz, 1H), 7.90 (d, J = 4.6 Hz, 1H), 7.35 (s, 1H), 7.28-7.12 (m, 3H), 6.89 (s, 1H), 6.01 (s, 1H), 5.76 (t, J = 7.3 Hz, 1H), 4.54-4.38 (m, 1H), 4.34 (dd, J = 13.0, 3.6 Hz, 1H), 4.22-4.08 (m, 1H), 4.04-3.97 (m, 1H), 3.96 (s, 3H), 3.89 (s, 3H), 3.83 (d, J = 12.0 Hz, 1H), 3.68 (d, J = 13.0 Hz, 1H), 3.44-3.34 (m, 4H), 3.30-3.20 (m, 1H), 3.12-3.00 (m, 1H), 2.76 (t, J = 11.9 Hz, 2H), 2.52-2.39 (m, 1H), 2.26 (d, J = 11.6 Hz, 2H), 1.91 (d, J = 11.9 Hz, 2H); 701.3. [M + H]⁺ |
| 74 | 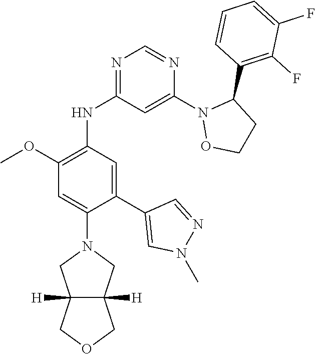 [SMILES: COc1cc(N2C[C@H]3COC[C@H]3C2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N-(2-methoxy-5-(1- methyl-1H-pyrazol-4-yl)-4- ((3aR,6a S)-tetrahydro-1H- furo[3,4-c]pyrrol-5(3H)- yl)phenyl)pyrimidin-4-amine | 576.3 [M + H]⁺ |
| 75 | 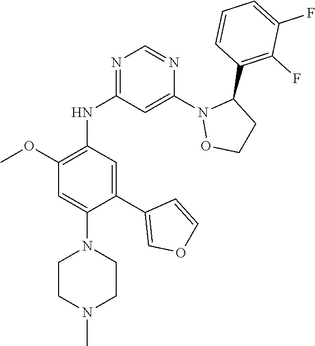 [SMILES: COc1cc(N2CCN(C)CC2)c(-c2ccoc2)cc1Nc1cc(N2OCC[C@@H]2c2cccc(F)c2F)ncn1] | (R)-6-(3-(2,3- difluorophenyl)isoxazolidin- 2-yl)-N-(5-(furan-3-yl)-2- methoxy-4-(4- methylpiperazin-1- yl)phenyl)pyrimidin-4-amine | ¹H NMR (400 MHz, Methanol-d₄) δ 8.11 (d, J = 1.0 Hz, 1H), 7.94 (dd, J = 1.6, 0.8 Hz, 1H), 7.57 (s, 1H), 7.50 (t, J = 1.7 Hz, 1H), 7.30 (t, J = 7.1 Hz, 1H), 7.16-7.09 (m, 2H), 6.86 (dd, J = 1.9, 0.8 Hz, 1H), 6.83 (s, 1H), 6.34 (d, J = 1.0 Hz, 1H), 5.76. (dd, J = 8.8, 4.9 Hz, 1H), 4.12 (td, J = 8.0, 4.1 Hz, 1H), 3.88 (s, 3H), 2.97 (s, 4H), 2.84 (ddt, J = 12.2, 8.0, 4.1 Hz, 2H), 2.59 (s, 4H), 2.35 (s, 3H), 2.29-2.20 (m, 2H); 549.4 [M + H]⁺ |
| 76 | 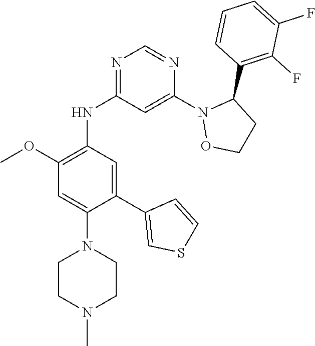 [SMILES: COc1cc(N2CCN(C)CC2)c(-c2ccsc2)cc1Nc1cc(N2OCC[C@@H]2c2cccc(F)c2F)ncn1] | (R)-6-(3-(2,3- difluorophenyl)isoxazolidin- 2-yl)-N-(2-methoxy-4-(4- methylpiperazin-1-yl)-5- (thiophen-3- yl)phenyl)pyrimidin-4-amine | ¹H NMR (400 MHz, Chloroform-d) δ 8.32 (s, 1H), 7.78 (s, 1H), 7.54-7.51 (m, 2H), 7.35-7.30 (m, 2H), 7.09-7.01 (m, 2H), 6.81 (s, 1H), 6.68 (s, 1H), 6:53 (s, 1H), 5.93-5.88 (m, 1H), 4.13- 4.08 (m, 1H), 3.92-3.85 (m, 4H), 2.93-2.87 (m, 4H), 2.84-2.78 (m, 1H), 2.51-2.39 (m, 4H), 2.35-2.26 (m, 4H).; 565.3 [M + H]⁺ |
| 77 | 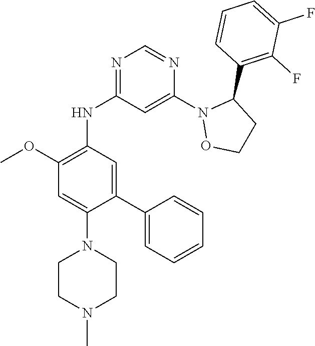 [SMILES: COc1cc(N2CCN(C)CC2)c(-c2ccccc2)cc1Nc1cc(N2OCC[C@@H]2c2cccc(F)c2F)ncn1] | (R)-6-(3-(2,3- difluorophenyl)isoxazolidin- 2-yl)-N-(4-methoxy-6-(4- methylpiperazin-1-yl)-[1,1′- biphenyl]-3-yl)pyrimidin-4- amine | 559.3 [M + H]⁺ |
| 78 | 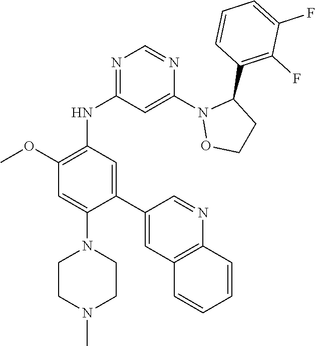 [SMILES: COc1cc(N2CCN(C)CC2)c(-c2cnc3ccccc3c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-4-(4- methylpiperazin-1-yl)-5- (quinolin-3- yl)phenyl)pyrimidin-4-amine | ¹H NMR (400 MHz, DMSO-d₆) δ 9.21 (d, J = 2.2 Hz, 1H), 8.74 (s, 1H), 8.41 (d, J = 2.1 Hz, 1H), 8.16 (s, 1H), 8.08-7.95 (m, 2H), 7.76-7.72 (m, 2H), 7.61 (ddd, J = 8.2, 6.8, 1.2 Hz, 1H), 7.42-7.25 (m, 2H), 7.24-7.14 (m, 1H), 6.88 (s, 1H), 6.43 (s, 1H), 5.75 (dd, J = 8.8, 5.0 Hz, 1H), 4.19 (td, J = 7.9, 3.7 Hz, 1H), 3.91 (s, 3H), 3.87-3.83 (m, 1H), 2.85-2.81 (m, 4H), 2.80-2.77 (m, 1H), 2.26-2.19 (m, 4H), 2.12 (s, 3H).; 610.3 [M + H]⁺ |
| 79 | 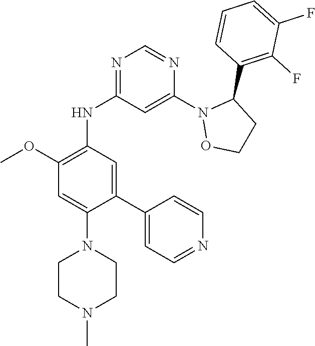 [SMILES: COc1cc(N2CCN(C)CC2)c(-c2ccncc2)cc1Nc1cc(N2OCC[C@@H]2c2cccc(F)c2F)ncn1] | (R)-6-(3-(2,3- difluorophenyl)isoxazolidin- 2-yl)-N-(2-methoxy-4-(4- methylpiperazin-1-yl)-5- (pyridin-4- yl)phenyl)pyrimidin-4-amine | ¹H NMR (400 MHz, DMSO-d₆) δ 8.70 (s, 1H), 8.57 (d, J = 5.9 Hz, 2H), 8.15 (s, 1H), 7.62 (d, J = 5.9 Hz, 3H), 7.36-7.27 (m, 2H), 7.22-7.17 (m, 1H), 6.80 (s, 1H), 6.44-6.35 (m, 1H), 3.88 (s, 3H), 3.84-3.81 (m, 1H), 2.83-2.79 (m, J = 4.4 Hz, 4H), 2.71-2.61 (m, 2H), 2.32-2.25 (m, 4H), 2.24-2.18 (m, 2H), 2.16 (s, 3H).; 560.3 [M + H]⁺ |
| 80 | 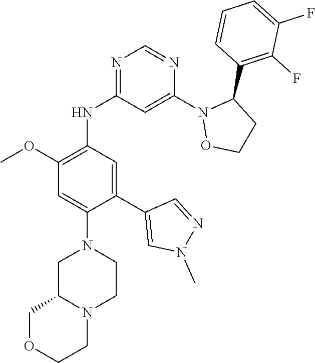 [SMILES: COc1cc(N2CCN3CCOC[C@@H]3C2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N-(4-((S)- hexahydropyrazino[2,1- c][1,4]oxazin-8(1H)-yl)-2- methoxy-5-(1-methyl-1H- pyrazol-4- yl)phenyl)pyrimidin-4-amine | 605.3 [M + H]⁺ |
| 81 | 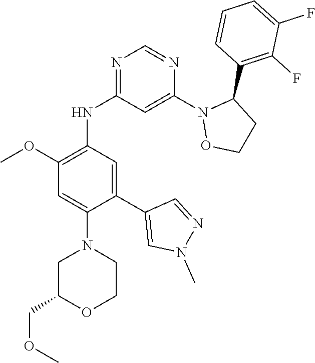 [SMILES: COC[C@@H]1CN(c2cc(OC)c(Nc3cc(N4OCC[C@@H]4c4cccc(F)c4F)ncn3)cc2-c2cnn(C)c2)CCO1] | 6-((R)-3-(2,3- difluorophenyl)isoxazolidin- 2-yl)-N-(2-methoxy-4-((S)-2- (methoxymethyl)morpholino)- 5-(1-methyl-1H-pyrazol-4- yl)phenyl)pyrimidin-4-amine | 594.3 [M + H]⁺ |
| 82 | 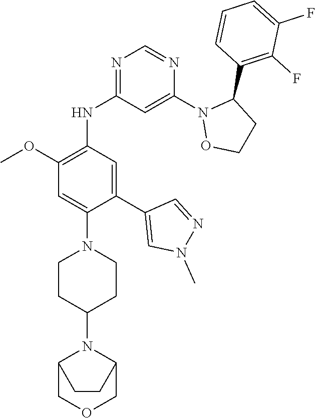 [SMILES: COc1cc(N2CCC(N3C4CCC3COC4)CC2)c(-c2cnn(C)c2)cc1Nc1cc(N2OCC[C@@H]2c2cccc(F)c2F)ncn1] | N-(4-(4-((1 R,5S)-3-oxa-8- azabicyclo[3.2.1]octan-8- yl)piperidin-1-yl)-2- methoxy-5-(1-methyl-1H- pyrazol-4-yl)phenyl)-6-((R)- 3-(2,3- difluorophenyl)isoxazolidin- 2-yl)pyrimidin-4-amine | 659.3 [M + H]⁺ |
| 83 | 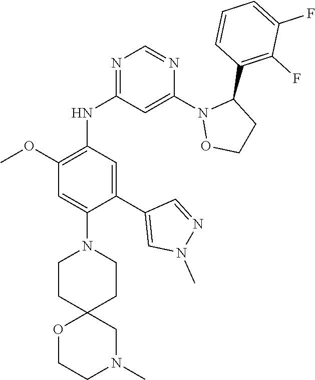 [SMILES: COc1cc(N2CCC3(CC2)CN(C)CCO3)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)- N-(2-methoxy-5-(1- methyl-1H-pyrazol-4-yl)-4- (4-methyl-1-oxa-4,9- diazaspiro[5.5]undecan-9- yl)phenyl)pyrimidin-4-amine | ¹H NMR (400 MHz, CDCl₃) δ 8.31 (s, 1H), 7.93 (s, 1H), 7.82 (s, 1H), 7.73 (s, 1H), 7.33 (d, J = 8.3 Hz, 1H), 7.05 (dd, J = 7.5, 4.7 Hz, 3H), 6.76 (s, 1H), 6.49 (s, 1H), 5.91 (dd, J = 8.4, 4.6 Hz, 1H), 4.11 (dd, J = 11.9, 7.7 Hz, 1H), 3.95 (s, 3H), 3.93-3.90 (m, 1H), 3.88 (s, 3H), 3.79- 3.75 (m, 2H), 2.94-2.83 (m, 4H), 2.39 (s, 2H), 2.29 (s, 2H), 2.26 (s, 3H), 2.09-1.98 (m, 4H), 1.66 (dd, ) = 16.6, 7.1 Hz, 2H); 633.4 [M + H]⁺ |
| 84 | 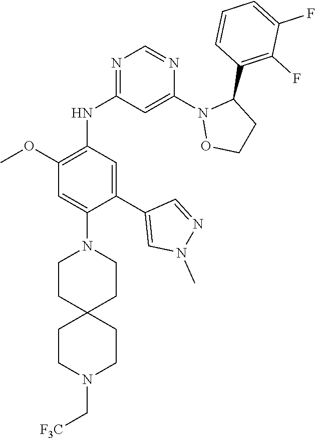 [SMILES: COc1cc(N2CCC3(CCN(CC(F)(F)F)CC3)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- (9-(2,2,2-trifluoroethyl)-3,9- diazaspiro[5.5]undecan-3- yl)phenyl)pyrimidin-4-amine | ¹H NMR (400 MHz, DMSO-d₆) δ 8.15 (s, 1H), 8.05 (s, 1H), 7.99 (s, 1H), 7.85 (s, 1H), 7.78 (s, 1H), 7.20 (s, 2H), 7.15-7.13 (m, 1H), 6.72 (s, 1H), 5.76-5.73 (m, 1H), 5.33 (s, 2H), 3.83-3.82 (m, 4H), 3.82-3.81 (m, 4H), 2.80-2.79 (m, 3H), 2.60-2.59 (m, 4H), 2.23-2.21 (m, J = 4.4 Hz, 4H), 1.53-1.52 (m, 8H).; 699.3 [M + H]⁺ |
| 85 | 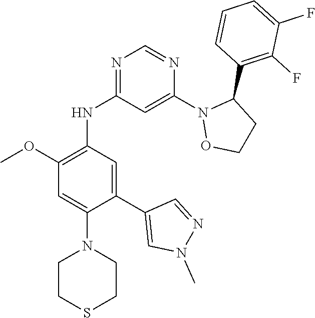 [SMILES: COc1cc(N2CCSCC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- thiomorpholinophenyl)pyri- midin-4-amine | ¹H NMR (400 MHz, Methanol-d₄) δ 8.03 (s, 1H), 7.90 (s, 1H), 7.79 (s, 1H), 7.50 (s, 1H), 7.25-7.19 (m, 1H), 7.11-6.99 (m, 2H), 6.75 (s, 1H), 6.27 (s, 1H), 5.71-5.66 (m, 1H), 4.07-4.02 (m, 1H), 3.87-3.80 (m, 4H), 3.79 (s, 3H), 3.07- 3.02 (m, 4H), 2.81-2.72 (m, 1H), 2.67-2.61 (m, 4H), 2.22-2.13 (m, 1H); 566.3 [M + H]⁺ |
| 86 | 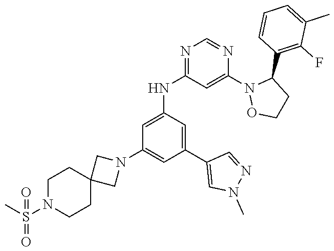 | (R)-6-(3-(2,3- difluorophenyl)isoxazolidin- 2-yl)-N-(3-(1-methyl-1H- pyrazol-4-yl)-5-(7- (methylsulfonyl)-2,7- diazaspiro[3.5]nonan-2- yl)phenyl)pyrimidin-4-amine | 637.3 [M + H]⁺ |
| 87 | 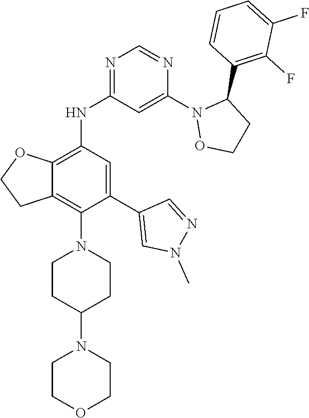 [SMILES: Cn1cc(-c2cc(Nc3cc(N4OCC[C@@H]4c4cccc(F)c4F)ncn3)c3c(c2N2CCC(N4CCOCC4)CC2)CCO3)cn1] | (R)-6-(3-(2,3- difluorophenyl)isoxazolidin- 2-yl)-N-(5-(1-methyl-1H- pyrazol-4-yl)-4-(4- morpholinopiperidin-1-yl)- 2,3-dihydrobenzofuran-7- yl)pyrimidin-4-amine | 645.3 [M + H]⁺ |
| 88 | 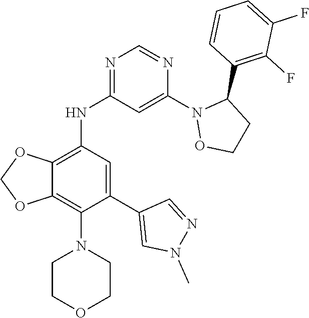 [SMILES: Cn1cc(-c2cc(Nc3cc(N4OCC[C@@H]4c4cccc(F)c4F)ncn3)c3c(c2N2CCOCC2)OCO3)cn1] | (R)-6-(3-(2,3- difluorophenyl)isoxazolidin- 2-yl)-N-(6-(1-methyl-1H- pyrazol-4-yl)-7- morpholinobenzo[d][1,3]dio- xol-4-yl)pyrimidin-4-amine | 564.3 [M + H]⁺ |
| 89 | 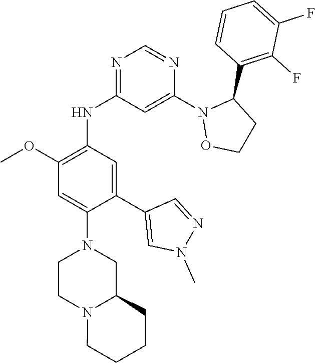 [SMILES: COc1cc(N2CCN3CCCC[C@@H]3C2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N-(2-methoxy-5-(1- methyl-1H-pyrazol-4-yl)-4- ((R)-octahydro-2H- pyrido[1,2-a]pyrazin-2- yl)phenyl)pyrimidin-4-amine | 603.3 [M + H]⁺ |
| 90 | 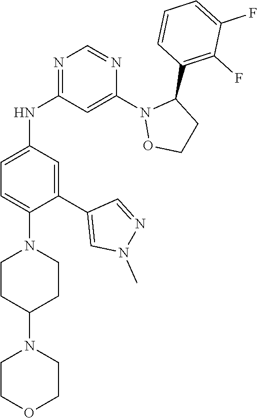 [SMILES: Cn1cc(-c2cc(Nc3cc(N4OCC[C@@H]4c4cccc(F)c4F)ncn3)ccc2N2CCC(N3CCOCC3)CC2)cn1] | (R)-6-(3-(2,3- difluorophenyl)isoxazolidin- 2-yl)-N-(3-(1-methyl-1H- pyrazol-4-yl)-4-(4- morpholinopiperidin-1- yl)phenyl)pyrimidin-4-amine | 603.3 [M + H]⁺ |
| 91 | 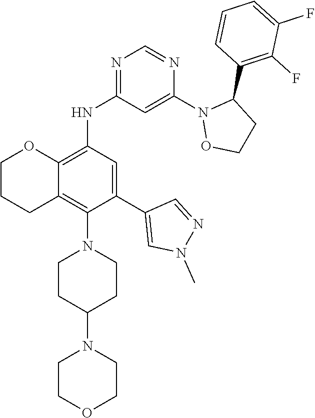 [SMILES: Cn1cc(-c2cc(Nc3cc(N4OCC[C@@H]4c4cccc(F)c4F)ncn3)c3c(c2N2CCC(N4CCOCC4)CC2)CCCO3)cn1] | (R)-6-(3-(2,3- difluorophenyl)isoxazolidin- 2-yl)-N-(6-(1-methyl-1H- pyrazol-4-yl)-5-(4- morpholinopiperidin-1- yl)chroman-8-yl)pyrimidin- 4-amine | 659.3 [M + H]⁺ |
| 92 | 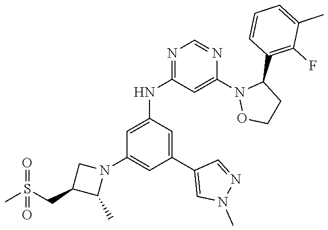 | 6-((R)-3-(2,3- difluorophenyl)isoxazolidin- 2-yl)- N-(3-(1-methyl-1H- pyrazol-4-yl)-5-((2R,35)-2- methyl-3- ((methylsulfonyl)methyl)azeti- din-1-yl)phenyl)pyrimidin- 4-amine | 596.3 [M + H]⁺ |
| 93 | 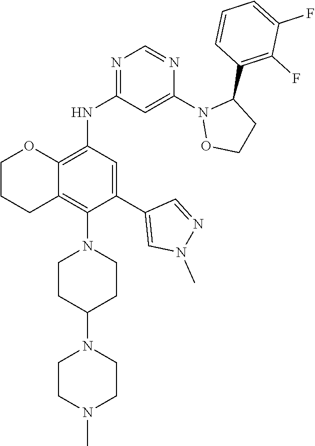 [SMILES: CN1CCN(C2CCN(c3c(-c4cnn(C)c4)cc(Nc4cc(N5OCC[C@@H]5c5cccc(F)c5F)ncn4)c4c3CCCO4)CC2)CC1] | (R)-6-(3-(2,3- difluorophenyl)isoxazolidin- 2-yl)- N-(6-(1-methyl-1H- pyrazol-4-yl)-5-(4-(4- methylpiperazin-1- yl)piperidin-1-yl)chroman-8- yl)pyrimidin-4-amine | 672.3 [M + H]⁺ |
| 94 | 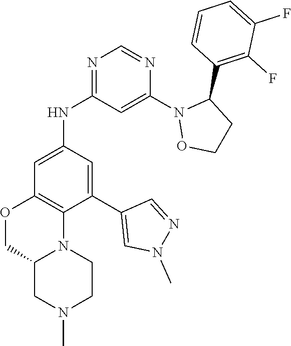 [SMILES: CN1CCN2c3c(cc(Nc4cc(N5OCC[C@@H]5c5cccc(F)c5F)ncn4)cc3-c3cnn(C)c3)OC[C@H]2C1] | (R)-N-(6-((R)-3-(2,3- difluorophenyl)isoxazolidin- 2-yl)pyrimidin-4-yl)-3- methyl-10-(1-methyl-1H- pyrazol-4-yl)-1,2,3,4,4a,5- hexahydrobenzo[b]pyrazino [1,2-d][1,4]oxazin-8-amine | 561.3 [M + H]⁺ |
| 95 | 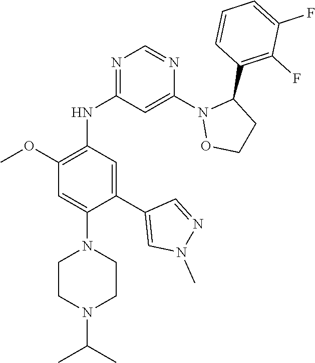 [SMILES: COc1cc(N2CCN(C(C)C)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(4-(4- isopropylpiperazin-1-yl)-2- methoxy-5-(1-methyl-1H- pyrazol-4- yl)phenyl)pyrimidin-4-amine | 591.3 [M + H]⁺ |
| 96 | 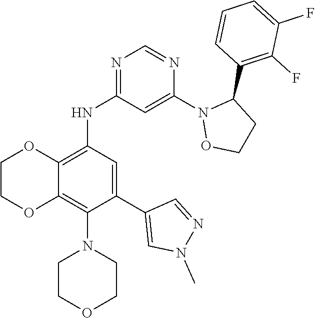 [SMILES: Cn1cc(-c2cc(Nc3cc(N4OCC[C@@H]4c4cccc(F)c4F)ncn3)c3c(c2N2CCOCC2)OCCO3)cn1] | (R)-6-(3-(2,3- difluorophenyl)isoxazolidin- 2-yl)-N-(7-(1-methyl-1H- pyrazol-4-yl)-8-morpholino- 2,3- dihydrobenzo[b][1,4]dioxin- 5-yl)pyrimidin-4-amine | ¹H NMR (400 MHz, Methanol-d₄) δ 8.06 (s, 1H), 7.88 (s, 1H), 7.73 (s, 1H), 7.25-7.18 (m, 2H), 7.11-7.00 (m, 2H), 6.35 (s, 1H), 5.72-5.66 (m, 1H), 4.48-4.46 (m, 4H), 4.26-4.20 (m, 4H), 4.10-4.03 (m, 1H), 3.89-3.80 (m, 4H), 3.69- 3.60 (m, 4H), 2.81-2.74 (m, 1H), 2.21-2.13 (m, 1H); 578.3 [M + H]⁺ |
| 97 | 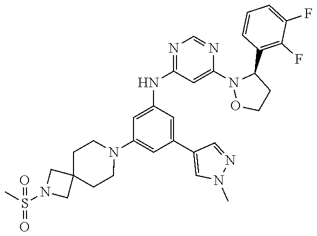 [SMILES: Cn1cc(-c2cc(Nc3cc(N4OCC[C@@H]4c4cccc(F)c4F)ncn3)cc(N3CCC4(CC3)CN(S(C)(=O)=O)C4)c2)cn1] | (R)-6-(3-(2,3- difluorophenyl)isoxazolidin- 2-yl)-N-(3-(1-methyl-1H- pyrazol-4-yl)-5-(2- (methylsulfonyl)-2,7- diazaspiro[3.5]nonan-7- yl)phenyl)pyrimidin-4-amine | 637.3 [M + H]⁺ |
| 98 | 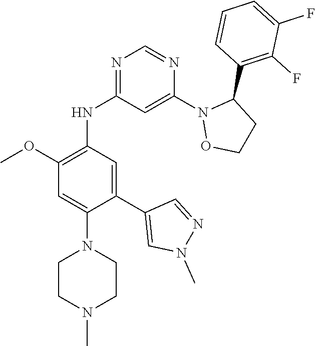 [SMILES: COc1cc(N2CCN(C)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-N-pyrazol-4-yl)-4- (4-methylpiperazin-1- yl)phenyl)pyrimidin-4-amine | ¹H NMR (400 MHz, Methanol-d₄) δ 8.03 (s, 1H), 7.88 (s, 1H), 7.81 (s, 1H), 7.49 (s, 1H), 7.26-7.19 (m, 1H), 7.11-6.99 (m, 2H), 6.75 (s, 1H), 6.26 (s, 1H), 5.72-5.65 (m, 1H), 4.08-4.00 (m, 1H), 3.88-3.76 (m, 7H), 2.92-2.83 (m, 4H), 2.80-2.70 (m, 1H), 2.58-2.42 (m, 4H), 2.27 (s, 3H), 2.22-2.12 (m, 1H); 563.3 [M + H]⁺ |
| 99 | 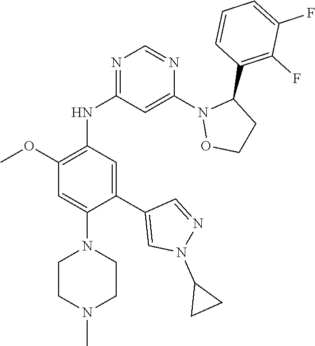 [SMILES: COc1cc(N2CCN(C)CC2)c(-c2cnn(C3CC3)c2)cc1Nc1cc(N2OCC[C@@H]2c2cccc(F)c2F)ncn1] | (R)-N-(5-(1-cyclopropyl-1H- pyrazol-4-yl)-2-methoxy-4- (4-methylpiperazin-1- yl)phenyl)-6-(3-(2,3- difluorophenyl)isoxazolidin- 2-yl)pyrimidin-4-amine | ¹H NMR (400 MHz, DMSO) δ 8.64 (s, 1H), 8.16 (d, J = 1.0 Hz, 1H), 8.12 (s, 1H), 7.84 (s, 1H), 7.60 (s, 1H), 7.40-7.25 (m, 2H), 7.23-7.16 (m, 1H), 6.80 (s, 1H), 6.28 (s, 1H), 5.75 (dd, J = 8.8, 5.1 Hz, 1H), 4.17 (td, J = 7.9, 3.7 Hz, 1H), 3.82 (s, 4H), 3.75 (tt, J = 7.4, 3.9 Hz, 1H), 2.89-2.75 (m, 5H), 2.44 (s, 4H), 2.27-2.14 (m, 4H), 1.05 (ddd, J = 7.3, 6.1, 3.8 Hz, 2H), 1.02- 0.94 (m, 2H) .; 589.3 [M + H]⁺ |
| 100 | 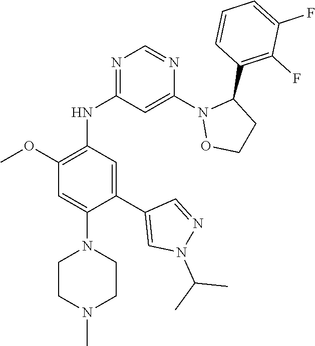 [SMILES: COc1cc(N2CCN(C)CC2)c(-c2cnn(C(C)C)c2)cc1Nc1cc(N2OCC[C@@H]2c2cccc(F)c2F)ncn1] | (R)-6-(3-(2,3- difluorophenyl)isoxazolidin- 2-yl)-N-(5-(1-isopropyl-1H- pyrazol-4-yl)-2-methoxy-4- (4-methylpiperazin-1- yl)phenyl)pyrimidin-4-amine | 591.3 [M + H]⁺ |
| 101 | 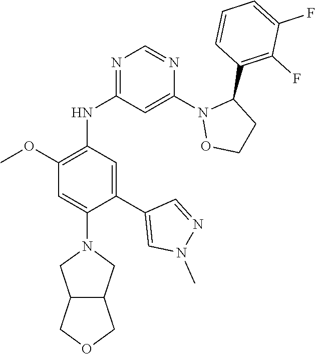 [SMILES: COc1cc(N2CC3COCC3C2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N-(2-methoxy-5-(1- methyl-1H-pyrazol-4-yl)-4- (tetrahydro-1H-furo[3,4- c]pyrrol-5(3H)- yl)phenyl)pyrimidin-4-amine | 576.3 [M + H]⁺ |
| 102 | 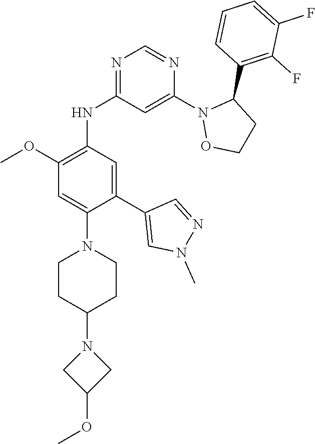 [SMILES: COc1cc(N2CCC(N3CC(OC)C3)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-4-(4-(3- methoxyazetidin-1- yl)piperidin-1-yl)-5-(1- methyl-1H-pyrazol-4- yl)phenyl)pyrimidin-4-amine | ¹H NMR (400 MHz, CDCl₃) δ 8.28 (s, 1H), 7.98 (s, 1H), 7.78 (s, 1H), 7.68 (s, 1H), 7.44 (s, 1H), 7.31 (d, J = 8.5 Hz, 1H), 7.09-7.02 (m, 2H), 6.69 (s, 1H), 6.46 (s, 1H), 5.90 (dd, J = 8.7, 4.7 Hz, 1H), 4.21-4.07 (m, 2H), 3.95 (s, 3H), 3.91- 3.85 (m, 6H), 3.29 (s, 3H), 3.19 (d, J = 11.5 Hz, 2H), 3.13-3.07 (m, 2H), 2.88-2.77 (m, 1H), 2.57 (dd, J = 21.1, 9.6 Hz, 2H), 2.33 (ddd, J = 13.6, 12.3, 7.8 Hz, 2H), 1.81 (d, J = 12.0 Hz, 2H), 1.58 (dd, J = 23.5, 11.7 Hz, 2H); 633.3 [M + H]⁺ |
| 103 | 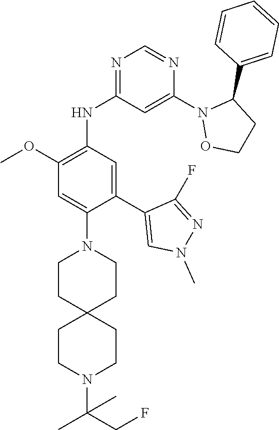 [SMILES: COc1cc(N2CCC3(CC2)CCN(C(C)(C)CF)CC3)c(-c2cn(C)nc2F)cc1Nc1cc(N2OCC[C@@H]2c2ccccc2)ncn1] | (R)-N-(5-(3-fluoro-1-methyl- 1H-pyrazol-4-yl)-4-(9-(1- fluoro-2-methylpropan-2- yl)-3,9- diazaspiro[5.5]undecan-3- yl)-2-methoxyphenyl)-6-(3- phenylisoxazolidin-2- yl)pyrimidin-4-amine | ¹H NMR (400 MHz, CDCl₃) δ 8.32 (d, J = 3.9 Hz, 1H), 7.82 (s, 1H), 7.73 (s, 1H), 7.45 (d, J = 7.5 Hz, 2H), 7.34 (t, J = 7.6 Hz, 2H), 7.24 (d, J = 7.3 Hz, 1H), 6.90 (s, 1H), 6.72 (s, 1H), 6.60 (s, 1H), 5.69 (dd, J = 8.5, 4.6 Hz, 1H), 4.20- 4.13 (m, 1H), 3.95 (q, J = 7.9 Hz, 1H), 3.88 (d, J = 3.7 Hz, 3H), 3.82 (s, 3H), 2.85 (ddd, J = 17.0, 11.3, 5.4 Hz, 4H), 2.77-2.71 (m, 1H), 2.43-2.36 (m, 1H), 1.65-1.37 (m, 14H), 1.25 (s, 6H); 673.5 [M + H]⁺ |
| 104 | 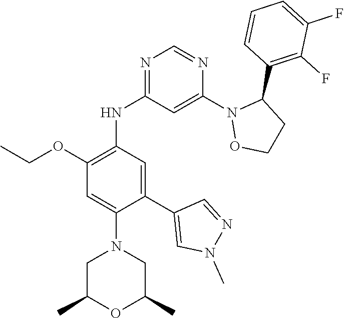 [SMILES: CCOc1cc(N2C[C@@H](C)O[C@@H](C)C2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N-(4-((25,6R)-2,6- dimethylmorpholino)-2- ethoxy-5-(1-methyl-1H- pyrazol-4- yl)phenyl)pyrimidin-4-amine | ¹H NMR (400 MHz, DMSO) δ 8.54 (s, 1H), 8.17 (d, J = 1.0 Hz, 1H), 8.09 (s, 1H), 7.82 (s, 1H), 7.65 (s, 1H), 7.41-7.25 (m, 2H), 7.25-7.15 (m, 1H), 6.78 (s, 1H), 6.33 (s, 1H), 5.79-5.73 (m, 1H), 4.22-4.14 (m, 1H), 4.10 (q, J = 7.0 Hz, 2H), 3.86 (s, 3H), 3.85-3.74 (m, 3H), 2.91 (d, J = 11.4 Hz, 2H), 2.86-2.77 (m, 1H), 2.37-2.30 (m, 2H), 2.26-2.17 (m, 1H), 1.30 (t, J = 7.0 Hz, 3H), 1.12-1.02 (m, 6H). 592.4 [M + H]⁺ |
| 105 | 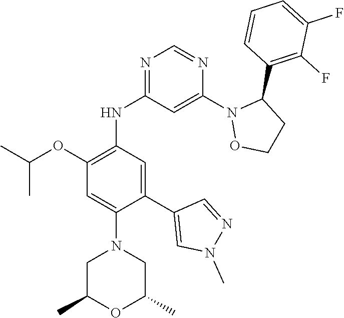 [SMILES: CC(C)Oc1cc(N2C[C@H](C)O[C@@H](C)C2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)-N--(4-((2S,6S)-2,6- dimethylmorpholino)-2- isopropoxy-5-(1-methyl-1H- pyrazol-4- yl)phenyl)pyrimidin-4-amine | ¹H NMR (400 MHz, DMSO-d₆) δ 8.47 (s, 1H), 8.17 (s, 1H), 8.10 (s, 1H), 7.82 (s, 1H), 7.67 (s, 1H), 7.41-7.24 (m, 2H), 7.24-7.15 (m, 1H), 6.78 (s, 1H), 6.35 (s, 1H), 5.76 (dd, J = 8.8, 5.1 Hz, 1H), 4.61 (p, J = 6.1 Hz, 1H), 4.17 (td, J = 7.8, 3.7 Hz, 1H), 3.86 (s, 3H), 3.84 (d, J = 8.1 Hz, 1H), 3.82-3.74 (m, 2H), 2.94-2.86 (m, 2H), 2.85-2.77 (m, 1H), 2.37-2.27 (m, 2H), 2.25- 2.16 (m, 1H), 1.25 (dd, J = 6.0, 3.5 Hz, 6H), 1.07 (dd, J = 6.3, 2.7 Hz, 6H).; 606:4 |
| 106 | 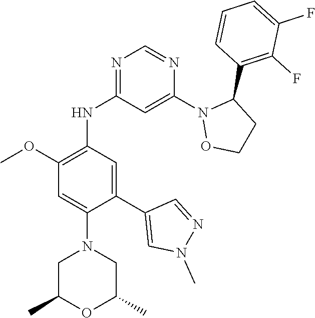 [SMILES: COc1cc(N2C[C@H](C)O[C@@H](C)C2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)- N-(4-((2S,6S)-2,6- dimethylmorpholino)-2- methoxy-5-(1-methyl-1H- pyrazol-4- yl)phenyl)pyrimidin-4-amine | ¹H NMR (400 MHz, DMSO-d₆) δ 8.64 (s, 1H), 8.16 (s, 1H), 7.95 (s, 1H), 7.73 (s, 1H), 7.60 (s, 1H), 7.40-7.25 (m, 2H), 7.23-7.18 (m, 1H), 6.80 (s, 1H), 6.33 (s, 1H), 5.77-5.73 (m, 1H), 4.17 (td, J = 8.0, 3.8 Hz, 1H), 4.03-3.99 (m, 2H), 3.88-3.87 (m, 1H), 3.86 (s, 3H), 3.83 (s, 3H), 2.93-2.89 (m, 2H), 2.83-2.79 (m, 1H), 2.49- 2.48 (m, 2H), 2.26-2.15 (m, 1H), 1.15 (s, 3H), 1.14 (s, 3H).; 578.3 [M + H]⁺ |
| 107 | 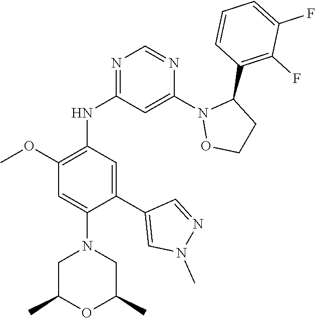 [SMILES: COc1cc(N2C[C@@H](C)O[C@@H](C)C2)c(-c2cnn(C)c2)cc1Nc1cc(N2OCC[C@@H]2c2cccc(F)c2F)ncn1] | 6-((R)-3-(2,3- difluorophenyl)isoxazolidin- 2-yl)- N-(4-((2S,6R)-2,6- dimethylmorpholino)-2- methoxy-5-(1-methyl-1H- pyrazol-4- yl)phenyl)pyrimidin-4-amine | ¹H NMR (400 MHZ, DMSO) δ 8.65 (s, 1H), 8.17 (d, J = 1.0 Hz, 1H), 8.10 (s, 1H), 7.86-7.81 (m, 1H), 7.66 (s, 1H), 7.40-7.26 (m, 2H), 7.24- 7.16 (m, 1H), 6.80 (s, 1H), 6.32 (s, 1H), 5.75 (dd, J = 8.9, 5.1 Hz, 1H), 4.17 (td, J = 8.0, 3.7 Hz, 1H), 3.84 (d, J = 13.9 Hz, 9H), 2.92 (d, J = 11.0 Hz, 2H), 2.86-2.76 (m, 1H), 2.40-2.30 (m, 2H), 2.26-2.16 (m, 1H), 1.07 (d, J = 5.9 Hz, 6H).; 578.3 [M + H]⁺ |
| 108 | 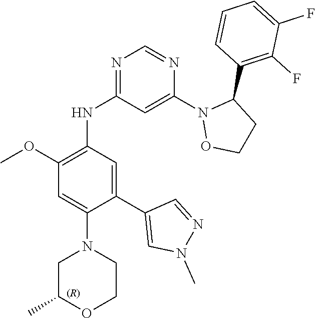 | 6-((R)-3-(2,3- difluorophenyl)isoxazolidin- 2-yl)- N-(2-methoxy-5-(1- methyl-1H-pyrazol-4-yl)-4- ((R)-2- methylmorpholino)phenyl)py- rimidin-4-amine | ¹H NMR (400 MHz, Methanol-d₄) δ 8.03 (s, 1H), 7.90 (s, 1H), 7.81 (s, 1H), 7.50 (s, 1H), 7.25-7.18 (m, 1H), 7.11-6.98 (m, 2H), 6.72 (s, 1H), 6.26 (s, 1H), 5.71-5.66 (m, 1H), 4.07-4.02 (m, 1H), 3.86-3.75 (m, 8H), 3.73-3.64 (m, 2H), 2.96-2.83 (m, 2H), 2.82-2.71 (m, 1H), 2.70- 2.64 (m, 1H), 2.40-2.34 (m, 1H), 2.22-2.13 (m, 1H), 1.04 (d, J = 6.3 Hz, 3H); 564.3 [M + H]⁺ |
| 109 | 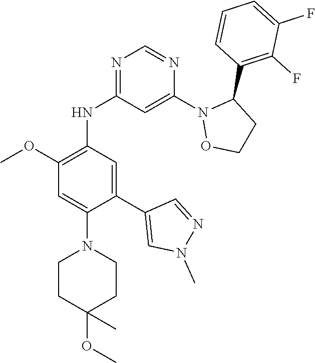 [SMILES: COc1cc(N2CCC(C)(OC)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-4-(4- methoxy-4-methylpiperidin- 1-yl)-5-(1-methyl-1H- pyrazol-4- yl)phenyl)pyrimidin-4-amine | ¹H NMR (400 MHz, DMSO) δ 8.63 (s, 1H), 8.16 (d, J = 1.0 Hz, 1H), 8.06 (s, 1H), 7.85 (d, J = 0.7 Hz, 1H), 7.57 (s, 1H), 7.40-7.26 (m, 2H), 7.20 (dt, J = 8.8, 6.4 Hz, 1H), 6.80 (s, 1H), 6.28 (s, 1H), 5.75 (dd, J = 8.9, 5.0 Hz, 1H), 4.17 (td, J = 7.9, 3.7 Hz, 1H), 3.84 (d, J = 17.7 Hz, 7H), 3.13 (s, 3H), 2.81 (d, J = 10.6 Hz, 5H), 2.26-2.15 (m, 1H), 1.75 (d, J = 13.0 Hz, 2H), 1.68-1.59 (m, 2H), 1.17 (s, 3H).; 592.4 [M + H]⁺ |
| 110 | 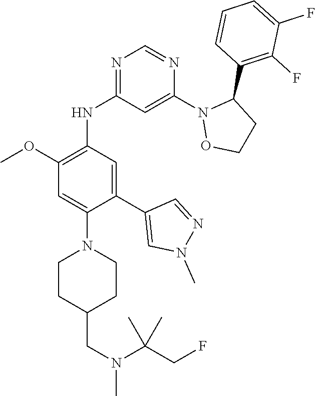 [SMILES: COc1cc(N2CCC(CN(C)C(C)(C)CF)CC2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)- N-(4-(4-(((1-fluoro-2- methylpropan-2- yl)(methyl)amino)methyl)pi- peridin-1-yl)-2-methoxy-5-(1- methyl-1H-pyrazol-4- yl)phenyl)pyrimidin-4-amine | ¹H NMR (400 MHz, DMSO) 8.73 (s, 1H), 8.17 (s, 1H), 8.04 (s, 1H), 7.88 (s, 1H), 7.59 (s, 1H), 7.40-7.31 (m, 1H), 7.31-7.25 (m, 1H), 7.25-7.15 (m, 1H), 6.78 (s, 1H), 6.29 (s, 1H), 5.80-5.71 (m, 1H), 4.23-4.13 (m, 1H), 3.91- 3.83 (m, 4H), 3.81 (s, 3H), 3.27-3.20 (m, 2H), 3.11 (d, J = 11.5 Hz, 2H), 2.92 (d, J = 4.5 Hz, 2H), 2.85-2.78 (m, 1H), 2.65-2.57 (m, 2H), 2.26-2.18 (m, 1H), 2.04-1.90 (m, 2H), 1.84- 1.76 (m, 2H), 1.51 (s, 3H), 1.46 (s, 3H), 1.43- 1.40 (m, 1H), 1.24 (s, 3H).; 665.5 [M + H]⁺ |
| 111 | 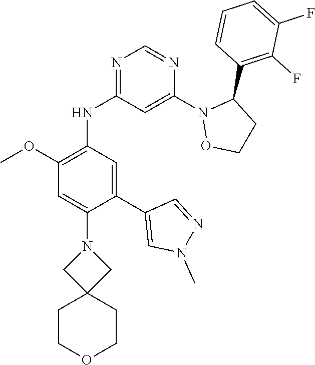 [SMILES: COc1cc(N2CC3(CCOCC3)C2)c(-c2cnn(C)c2)cc1Nc1cc(N2OCC[C@@H]2c2cccc(F)c2F)ncn1] | (R)-6-(3-(2,3- difluorophenyl)isoxazolidin- 2-yl)-N-(2-methoxy-5-(1- methyl-1H-pyrazol-4-yl)-4- (7-oxa-2- azaspiro[3.5]nonan-2- yl)phenyl)pyrimidin-4-amine | ¹H NMR (400 MHz, DMSO) δ 8.50 (s, 1H), 8.10 (d, J = 1.0 Hz, 1H), 7.70 (s, 1H), 7.45 (s, 1H), 7.41-7.24 (m, 2H), 7.22-7.16 (m, 1H), 7.13 (s, 1H), 6.26 (s, 1H), 6.14 (s, 1H), 5.78-5.69 (m, 1H), 4.19-4.11 (m, 1H), 3.85 (s, 3H), 3.83-3.76 (m, 4H), 3.50 (t, J = 5.2 Hz, 4H), 3.39 (s, 4H), 2.87- 2.74 (m, 1H), 2.26-2.14 (m, 1H), 1.67 (t, J = 5.2 Hz, 4H).; 590.4 [M + H]⁺ |
| 112 | 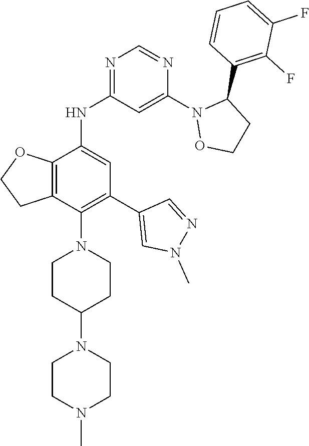 [SMILES: CN1CCN(C2CCN(c3c(-c4cnn(C)c4)cc(Nc4cc(N5OCC[C@@H]5c5cccc(F)c5F)ncn4)c4c3CCO4)CC2)CC1] | (R)-6-(3-(2,3- difluorophenyl)isoxazolidin- 2-yl)- N-(5-(1-methyl-1H- pyrazol-4-yl)-4-(4-(4- methylpiperazin-1- yl)piperidin-1-yl)-2,3- dihydrobenzofuran-7- yl)pyrimidin-4-amine | 658.4 [M + H]⁺ |
| 113 | 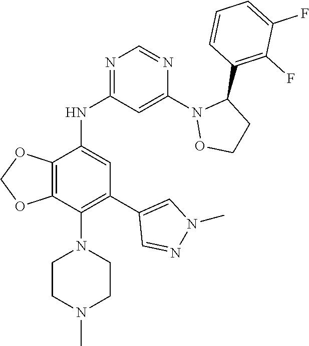 [SMILES: CN1CCN(c2c(-c3cnn(C)c3)cc(Nc3cc(N4OCC[C@@H]4c4cccc(F)c4F)ncn3)c3c2OCO3)CC1] | (R)-6-(3-(2,3- difluorophenyl)isoxazolidin- 2-yl)-N-(6-(1-methyl-1H- pyrazol-4-yl)-7-(4- methylpiperazin-1- yl)benzo[d][1,3]dioxol-4- yl)pyrimidin-4-amine | ¹H NMR (400 MHz, DMSO) δ 9.10 (s, 1H), 8.19 (s, 1H), 8.04 (s, 1H), 7.80 (s, 1H), 7.36 (dtd, J = 10.0, 7.9, 1.8 Hz, 1H), 7.29 (t, J = 7.0 Hz, 1H), 7.20 (td, J = 8.1, 4.7 Hz, 1H), 7.09 (s, 1H), 6.31 (s, 1H), 6.01 (d, J = 8.4 Hz, 2H), 5.78-5.72 (m, 1H), 4.20 (td, J = 7.9, 3.6 Hz, 1H), 3.89-3.81 (m, 1H), 3.86 (s, 3H), 2.98 (t, J = 4.8 Hz, 4H), 2.82 (ddt, J = 12.0, 8.1, 4.1 Hz, 1H), 2.39 (s, 4H), 2.26-2.17 (m, 1H), 2.22 (s, 3H).; 577.3 [M + H]⁺ |

### <Experimental Example 1> Evaluation of Inhibitory Activity on Proliferation of Ba/F3 Cell Overexpressing EGFR Mutations

The following experiment was performed to evaluate the inhibitory activity of the compounds according to the present invention on the proliferation of Ba/F3 cells expressing EGFR Del19/C797S (EGFR DC) and EGFR L858R/C797S (EGFR LC) mutations.

Ba/F3 cells were cultured using medium (RPMI-1640) containing 10% fetal bovine serum (FBS) and 5 ng/ml IL-3 (R&D Systems). The transduced Ba/F3 cells were cultured by adding 1 μg/ml puromycin (Invitrogen) to the same medium.

24 hours prior to treatment with compounds, 3,000 to 5,000 cells were seeded on a well plate (white clear bottom 96 well plate, Corning). The compounds were diluted in dimethyl sulfoxide (3-fold dilution, total of 12 concentrations) and injected at 1 μl each so that the final concentration was 0.2 nM to 5 μM. After 72 hours post treatment with compounds, living cells were stored at room temperature for 10 minutes using CellTiter-Glo luminescent cell-viability reagent (Promega), and the luminescence intensity thereof was measured using a reader (Synergy Neo, Biotek). Each test was repeated three times. The result values were calculated as a cell growth rate (%) compared to the control group. A graph was created using the GraphPad Prism version 8.3.0 program, and GI₅₀ values were calculated.

Table 2 below shows the evaluation results of the inhibitory activity on proliferation of Ba/F3 cells expressing EGFR Del19/C797S (EGFR DC) and EGFR L858R/C797S (EGFR LC) mutations.

TABLE 2

| Example | Ba/F3 DC | Ba/F3 LC |
|---|---|---|
| 1 | A | A |
| 10 | A | A |
| 14 | A | A |
| 16 | A | — |
| 17 | B | — |
| 28 | A | A |
| 30 | A | A |
| 31 | A | — |
| 32 | B | B |
| 41 | B | B |
| 42 | B | — |
| 43 | B | — |
| 44 | B | — |
| 45 | B | — |
| 46 | A | — |
| 47 | B | — |
| 50 | A | A |
| 55 | A | |
| 56 | A | |
| 59 | B | — |
| 60 | B | — |
| 61 | B | — |
| 62 | A | — |
| 63 | A | — |
| 64 | B | — |
| 66 | B | — |
| 67 | A | — |
| 70 | B | — |
| 71 | B | — |
| 73 | B | — |
| 75 | B | — |
| 76 | B | — |
| 78 | B | — |
| 79 | B | — |
| 83 | B | — |
| 85 | B | — |
| 96 | B | — |
| 98 | A | — |
| 99 | A | — |
| 102 | A | — |
| 103 | A | — |
| 104 | B | — |
| 105 | B | — |
| 106 | B | — |
| 107 | B | — |
| 108 | B | — |
| 109 | A | — |
| 110 | B | — |
| 111 | B | — |
| 112 | B | — |
| 113 | A | — |

(A: GI₅₀ < 100 nM; B: 100 nM ≤ GI₅₀ < 1000 nM; C: 1000 nM ≤ GI₅₀ < 10,000 nM; D: 10,000 nM ≤ GI₅₀)

As shown in Table 2 above, it could be appreciated that the compounds according to Examples of the present invention exhibited high inhibitory ability against overexpressing cell lines including EGFR Del19/C797S (EGFR DC) and EGFR L858R/C797S (EGFR LC) mutations, and the like.

As described above, the present invention has been described in detail through preferred Preparation Example, Examples, and Experimental Examples, but the scope of the present invention is not limited to the specific compounds according to Examples of the present invention and should be interpreted by the claims. Further, those skilled in the art should understand that many modifications and variations can be made without departing from the scope of the present invention.

1. A compound represented by the following Chemical Formula 1, a stereoisomer thereof, or a pharmaceutically acceptable salt thereof: 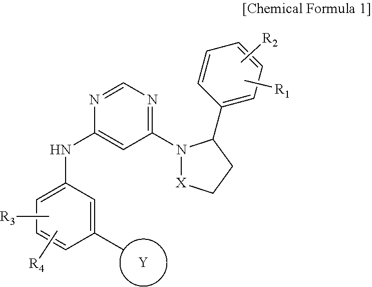
in Chemical Formula 1 above,
X is CH₂ or O;
R₁ and R₂ are each independently —H, —C₁₋₆alkyl, —C₁₋₆haloalkyl, or -halo;
R₃ is —H, —C₁₋₆alkyl, —C₁₋₆haloalkyl, —O—C₁₋₆alkyl, or —O—C₁₋₃alkyl-OH {wherein R₃ may be linked to a carbon in the phenyl ring to form a fused ring or linked to R₄ to form a fused ring};
R₄ is —H, —NH(C₁₋₆alkyl), —N(C₁₋₆alkyl)(C₁₋₆alkyl), -(4-6 membered heterocycloalkyl), or (7-11 membered heterobicycloalkyl) {wherein the —NH(C₁₋₆alkyl) or —N(C₁₋₆alkyl)(C₁₋₆alkyl) may be linked to the ring Y to form a fused ring; at least one H of the -(4-6 membered heterocycloalkyl) may be substituted with —C₁₋₆alkyl, —C₁₋₆haloalkyl, —(CH₂)n-O—C₁₋₆alkyl, (CH₂)n-NRₐR_{b}, —(CH₂)n-S(═O)₂—C₁₋₃alkyl, -halo, —(CH₂)n-(4-6 membered heterocycloalkyl), or —(CH₂)n-(7-11 membered heterobicycloalkyl), [here, at least one H of the —(CH₂)n-(4-6 membered heterocycloalkyl) or —(CH₂)n-(7-11 membered heterobicycloalkyl) ring may be substituted with —C₁₋₆alkyl, —C₁₋₆haloalkyl, —(CH₂)n-O—C₁₋₆alkyl, —(CH₂)n-C(═O)—C₁₋₃alkyl, —(CH₂)n-S(═O)₂—C₁₋₃alkyl, —(CH₂)nNRₐR_{b}, or -halo]; at least one H of the -(7-11 membered heterobicycloalkyl) may be substituted with —C₁₋₆alkyl, —C₁₋₆haloalkyl, —(CH₂)n-C(═O)—C₁₋₃alkyl, —(CH₂)n-C(═O)-(3-6 membered cycloalkyl), —(CH₂)n-S(═O)₂—C₁₋₃alkyl, —(CH₂)n-NRₐR_{b}, —O—C₁₋₆alkyl, -halo, or —(CH₂)n-(4-6 membered heterocycloalkyl) [here, at least one H of the —(CH₂)n-(4-6 membered heterocycloalkyl) or —(CH₂)n-C(═O)-(3-6 membered cycloalkyl) ring may be substituted with —C₁₋₆alkyl, —C₁₋₆haloalkyl, or -halo]};
n is 0, 1, 2, 3, or 4;
Rₐ and R_{b} are each independently —H, —C₁₋₆alkyl, —C₁₋₆haloalkyl, or -(4-6 membered heterocycloalkyl);
ring Y is a phenyl or 5-10 membered heteroaryl {wherein at least one H of the phenyl or 5-10 membered heteroaryl ring may be substituted with —C₁₋₆alkyl, —C₁₋₆hydroxyalkyl, —C₁₋₆haloalkyl, -(3-6 membered cycloalkyl), or -halo}.

2. The compound represented by Chemical Formula 1, the stereoisomer thereof, or the pharmaceutically acceptable salt thereof according to claim 1, wherein
X is CH₂ or O;
R₁ and R₂ are each independently —H or -halo;
R₃ is —H, —C₁₋₃haloalkyl, —O—C₁₋₃alkyl, or —O—C₁₋₃alkyl-OH {wherein R₃ may be linked to a carbon in the phenyl ring to form a fused ring, or linked to R₄ to form a fused ring};
R₄ is —H, —N(C₁₋₃alkyl)(C₁₋₃alkyl), -(4-6 membered heterocycloalkyl), or -(7-11 membered heterobicycloalkyl) {wherein the —N(C₁₋₃alkyl)(C₁₋₃alkyl) may be linked to the ring Y to form a fused ring; at least one H of the -(4-6 membered heterocycloalkyl) may be substituted with —C₁₋₆alkyl, —C₁₋₆haloalkyl, —(CH₂)n-O—C₁₋₃alkyl, —(CH₂)n-NRₐR_{b}, —(CH₂)n-S(═O)₂—C₁₋₃alkyl, -halo, —(CH₂)n-(4-6 membered heterocycloalkyl), or —(CH₂)n-(7-11 membered heterobicycloalkyl) [here, at least one H of the —(CH₂)n-(4-6 membered heterocycloalkyl) or —(CH₂)n-(7-11 membered heterobicycloalkyl) ring may be substituted with —C₁₋₃alkyl, —C₁₋₆haloalkyl, —(CH₂)n-O—C₁₋₃alkyl, —(CH₂)n-S(═O)₂—C₁₋₃alkyl, —(CH₂)nNRₐR_{b}, or -halo]; at least one H of the -(7-11 membered heterobicycloalkyl) may be substituted with —C₁₋₃alkyl, —C₁₋₆haloalkyl, —(CH₂)n-C(═O)—C₁₋₃alkyl, —(CH₂)n-C(═O)-(3-6 membered cycloalkyl), —(CH₂)n-S(═O)₂—C₁₋₃alkyl, —(CH₂)n-NRₐR_{b}, —O—C₁₋₃alkyl, -halo, or —(CH₂)n-(4-6 membered heterocycloalkyl) [here, at least one H of the —(CH₂)n-(4-6 membered heterocycloalkyl) or —(CH₂)n-C(═O)-(3-6 membered cycloalkyl) ring may be substituted with -halo]};
n is 0, 1, 2, or 3;
Rₐ and R_{b} are each independently —H, —C₁₋₃alkyl, —C₁₋₆haloalkyl, or -(4-6 membered heterocycloalkyl);
ring Y is a phenyl, 5-6 membered heteroaryl, or 9-10 membered heteroaryl {wherein at least one H of the phenyl, 5-6 membered heteroaryl, or 9-10 membered heteroaryl ring may be substituted with —C₁₋₃alkyl, —C₁₋₃hydroxyalkyl, -(3-6 membered cycloalkyl), or -halo}.

3. The compound represented by Chemical Formula 1, the stereoisomer thereof, or the pharmaceutically acceptable salt thereof according to claim 1, wherein

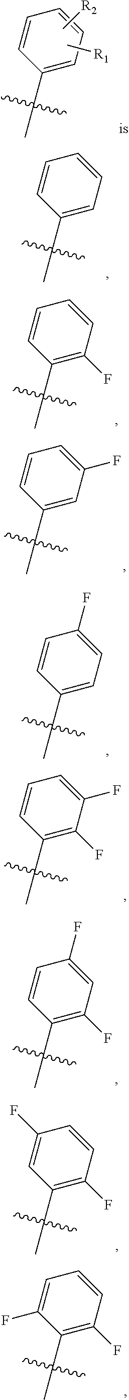

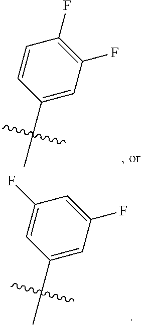 [SMILES: *c1ccc(F)c(F)c1 ; *c1cc(F)cc(F)c1]

4. The compound represented by Chemical Formula 1, the stereoisomer thereof, or the pharmaceutically acceptable salt thereof according to claim 1, wherein
ring Y is 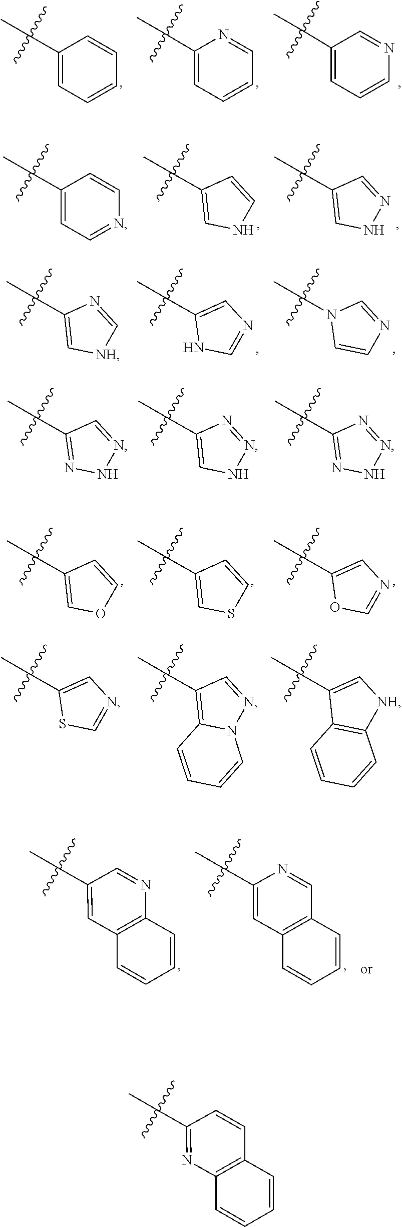
{wherein at least one H of the ring Y may be substituted with —C₁₋₃alkyl, —C₁₋₃hydroxyalkyl, —C₁₋₃haloalkyl, -(3-6 membered cycloalkyl), or -halo}.

5. A compound represented by the following Chemical Formula 2, a stereoisomer thereof, or a pharmaceutically acceptable salt thereof: 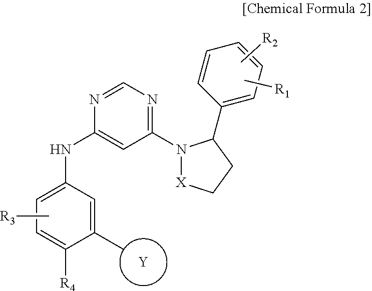
in Chemical Formula 2 above,
X is CH₂ or O;
R₁ and R₂ are each independently —H or -halo;
R₃ is —H, —C₁₋₃haloalkyl, —O—C₁₋₃alkyl, or —O—C₁₋₃alkyl-OH {wherein R₃ may be linked to a carbon in the phenyl ring to form a fused ring, or linked to R₄ to form a fused ring};
R₄ is —H, —N(C₁₋₃alkyl)(C₁₋₃alkyl), -(4-6 membered heterocycloalkyl), or -(7-11 membered heterobicycloalkyl) {wherein the —N(C₁₋₃alkyl)(C₁₋₃alkyl) may be linked to the ring Y to form a fused ring; at least one H of the -(4-6 membered heterocycloalkyl) may be substituted with —C₁₋₆alkyl, —C₁₋₆haloalkyl, —(CH₂)n-O—C₁₋₃alkyl, —(CH₂)n-NRₐR_{b}, —(CH₂)n-S(═O)₂—C₁₋₃alkyl, -halo, —(CH₂)n-(4-6 membered heterocycloalkyl), or —(CH₂)n-(7-11 membered heterobicycloalkyl) [here, at least one H of the —(CH₂)n-(4-6 membered heterocycloalkyl) or —(CH₂)n-(7-11 membered heterobicycloalkyl) ring may be substituted with —C₁₋₃alkyl, —C₁₋₆haloalkyl, —(CH₂)n-O—C₁₋₃alkyl, —(CH₂)n-S(═O)₂—C₁₋₃alkyl, —(CH₂)nNRₐR_{b}, or -halo]; at least one H of the -(7-11 membered heterobicycloalkyl) may be substituted with —C₁₋₃alkyl, —C₁₋₆haloalkyl, —(CH₂)n-C(═O)—C₁₋₃alkyl, —(CH₂)n-C(═O)-(3-6 membered cycloalkyl), —(CH₂)n-S(═O)₂—C₁₋₃alkyl, —(CH₂)n-NRₐR_{b}, —O—C₁₋₃alkyl, -halo, or —(CH₂)n-(4-6 membered heterocycloalkyl) [here, at least one H of the —(CH₂)n-(4-6 membered heterocycloalkyl) or —(CH₂)n-C(═O)-(3-6 membered cycloalkyl) ring may be substituted with -halo]};
n is 0, 1, 2, or 3;
Rₐ and R_{b} are each independently —H, —C₁₋₃alkyl, —C₁₋₆haloalkyl, or -(4-6 membered heterocycloalkyl);
ring Y is a phenyl, 5-6 membered heteroaryl, or 9-10 membered heteroaryl {wherein at least one H of the phenyl, 5-6 membered heteroaryl, or 9-10 membered heteroaryl ring may be substituted with —C₁₋₃alkyl, —C₁₋₃hydroxyalkyl, -(3-6 membered cycloalkyl), or -halo}.

6. A compound represented by the following Chemical Formula 3, a stereoisomer thereof, or a pharmaceutically acceptable salt thereof: 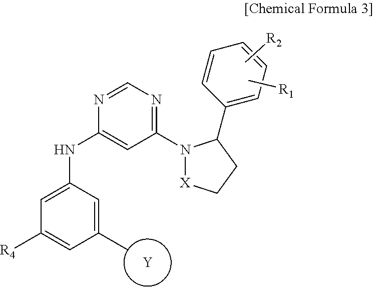
in Chemical Formula 3 above,
X is CH₂ or O;
R₁ and R₂ are each independently —H or -halo;
R₄ is -(4-6 membered heterocycloalkyl) or -(7-11 membered heterobicycloalkyl) {wherein at least one H of the -(4-6 membered heterocycloalkyl) or -(7-11 membered heterobicycloalkyl) ring may be substituted with —C₁₋₃alkyl, —C₁₋₃haloalkyl, —(CH₂)n-S(═O)₂—C₁₋₃alkyl, or -halo};
n is 0, 1, or 2;
ring Y is a 5 membered heteroaryl {wherein, at least one H of the 5 membered heteroaryl ring may be substituted with —C₁₋₃alkyl, or -halo}.

7. A compound selected from the group consisting of the following compounds, a stereoisomer thereof, or a pharmaceutically acceptable salt thereof:

| | |
|---|---|
| 1 | (R)-6-(3-(2,3-difluorophenyl)isoxazolidin-2-yl)-N-(5-(3-fluoro-1-methyl-1H- |
| | pyrazol-4-yl)-4-(9-(1-fluoro-2-methylpropan-2-yl)-3,9-diazaspiro[5.5]undecan-3- |
| | yl)-2-methoxyphenyl)pyrimidin-4-amine |
| 2 | (R)-6-(3-(2,3-difluorophenyl)isoxazolidin-2-yl)-N-(2-methoxy-5-(1-methyl-1H- |
| | pyrazol-4-yl)-4-(4-(4-(2-(methylsulfonyl)ethyl)piperazin-1-yl)piperidin-1- |
| | yl)phenyl)pyrimidin-4-amine |
| 3 | (R)-6-(3-(2,3-difluorophenyl)isoxazolidin-2-yl)-N-(4-(9-(1-fluoro-2- |
| | methylpropan-2-yl)-3,9-diazaspiro[5.5]undecan-3-yl)-2-methoxy-5-(2-methyl- |
| | 2H-tetrazol-5-yl)phenyl)pyrimidin-4-amine |
| 4 | (R)-6-(3-(2,4-difluorophenyl)isoxazolidin-2-yl)-N-(4-(9-(1-fluoro-2- |
| | methylpropan-2-yl)-3,9-diazaspiro[5.5]undecan-3-yl)-2-methoxy-5-(1-methyl- |
| | 1H-pyrazol-4-yl)phenyl)pyrimidin-4-amine |
| 5 | (R)-6-(3-(3,5-difluorophenyl)isoxazolidin-2-yl)-N-(4-(9-(1-fluoro-2- |
| | methylpropan-2-yl)-3,9-diazaspiro[5.5]undecan-3-yl)-2-methoxy-5-(1-methyl- |
| | 1H-pyrazol-4-yl)phenyl)pyrimidin-4-amine |
| 6 | (R)-6-(3-(3,4-difluorophenyl)isoxazolidin-2-yl)-N-(4-(9-(1-fluoro-2- |
| | methylpropan-2-yl)-3,9-diazaspiro[5.5]undecan-3-yl)-2-methoxy-5-(1-methyl- |
| | 1H-pyrazol-4-yl)phenyl)pyrimidin-4-amine |
| 7 | (R)-6-(3-(2,5-difluorophenyl)isoxazolidin-2-yl)-N-(4-(9-(1-fluoro-2- |
| | methylpropan-2-yl)-3,9-diazaspiro[5.5]undecan-3-yl)-2-methoxy-5-(1-methyl- |
| | 1H-pyrazol-4-yl)phenyl)pyrimidin-4-amine |
| 8 | (R)-N-(4-(9-(1-fluoro-2-methylpropan-2-yl)-3,9-diazaspiro[5.5]undecan-3-yl)-2- |
| | methoxy-5-(1-methyl-1H-pyrazol-4-yl)phenyl)-6-(3-(3- |
| | fluorophenyl)isoxazolidin-2-yl)pyrimidin-4-amine |
| 9 | (R)-N-(4-(9-(1-fluoro-2-methylpropan-2-yl)-3,9-diazaspiro[5.5]undecan-3-yl)-2- |
| | methoxy-5-(1-methyl-1H-pyrazol-4-yl)phenyl)-6-(3-(4- |
| | fluorophenyl)isoxazolidin-2-yl)pyrimidin-4-amine |
| 10 | (R)-N-(4-(9-(1-fluoro-2-methylpropan-2-yl)-3,9-diazaspiro[5.5]undecan-3-yl)-2- |
| | methoxy-5-(1-methyl-1H-pyrazol-4-yl)phenyl)-6-(3-phenylisoxazolidin-2- |
| | yl)pyrimidin-4-amine |
| 11 | (R)-6-(3-(2,3-difluorophenyl)isoxazolidin-2-yl)-N-(4-(4-(1-(1-fluoro-2- |
| | methylpropan-2-yl)azetidin-3-yl)piperidin-1-yl)-2-methoxy-5-(1-methyl-1H- |
| | pyrazol-4-yl)phenyl)pyrimidin-4-amine |
| 12 | (R)-6-(3-(2,3-difluorophenyl)isoxazolidin-2-yl)-N-(4-(3-(1-(1-fluoro-2- |
| | methylpropan-2-yl)piperidin-4-yl)azetidin-1-yl)-2-methoxy-5-(1-methyl-1H- |
| | pyrazol-4-yl)phenyl)pyrimidin-4-amine |
| 13 | (R)-6-(3-(2,3-difluorophenyl)isoxazolidin-2-yl)-N-(4-(1′-(1-fluoro-2- |
| | methylpropan-2-yl)-[3,3′-biazetidin]-1-yl)-2-methoxy-5-(1-methyl-1H-pyrazol-4- |
| | yl)phenyl)pyrimidin-4-amine |
| 14 | (R)-6-(3-(2,3-difluorophenyl)isoxazolidin-2-yl)-N-(4-(7-(1-fluoro-2- |
| | methylpropan-2-yl)-2,7-diazaspiro[3.5]nonan-2-yl)-2-methoxy-5-(1-methyl-1H- |
| | pyrazol-4-yl)phenyl)pyrimidin-4-amine |
| 15 | (R)-6-(3-(2,3-difluorophenyl)isoxazolidin-2-yl)-N-(2-methoxy-5-(1-methyl-1H- |
| | pyrazol-4-yl)-4-(4-((1-methylpiperidin-4-yl)methyl)piperazin-1- |
| | yl)phenyl)pyrimidin-4-amine |
| 16 | (R)-6-(3-(2,3-difluorophenyl)isoxazolidin-2-yl)-N-(5-(3-fluoro-1-methyl-1H- |
| | pyrazol-4-yl)-2-methoxy-4-morpholinophenyl)pyrimidin-4-amine |
| 17 | (R)-6-(3-(2,3-difluorophenyl)isoxazolidin-2-yl)-N-(5-(3-fluoro-1-methyl-1H- |
| | pyrazol-4-yl)-2-methoxy-4-(4-(oxetan-3-yl)piperidin-1-yl)phenyl)pyrimidin-4- |
| | amine |
| 18 | (R)-6-(3-(2,3-difluorophenyl)isoxazolidin-2-yl)-N-(5-(3-fluoro-1-methyl-1H- |
| | pyrazol-4-yl)-4-(4-((1-fluoro-2-methylpropan-2-yl)(methyl)amino)piperidin-1- |
| | yl)-2-methoxyphenyl)pyrimidin-4-amine |
| 19 | (R)-6-(2-(2,3-difluorophenyl)pyrrolidin-1-yl)-N-(4-(2-(1-fluoro-2-methylpropan- |
| | 2-yl)-2,7-diazaspiro[3.5]nonan-7-yl)-2-methoxy-5-(1-methyl-1H-pyrazol-4- |
| | yl)phenyl)pyrimidin-4-amine |
| 20 | N-(4-(7,7-difluorohexahydropyrrolo[1,2-a]pyrazin-2(1H)-yl)-2-methoxy-5-(1- |
| | methyl-1H-pyrazol-4-yl)phenyl)-6-((R)-2-(2,3-difluorophenyl)pyrrolidin-1- |
| | yl)pyrimidin-4-amine |
| 21 | (R)-N-(5-(3-chloro-1-methyl-1H-pyrazol-4-yl)-4-(9-(1-fluoro-2-methylpropan-2- |
| | yl)-3,9-diazaspiro[5.5]undecan-3-yl)-2-methoxyphenyl)-6-(3-(2,3- |
| | difluorophenyl)isoxazolidin-2-yl)pyrimidin-4-amine |
| 22 | (R)-6-(3-(2,3-difluorophenyl)isoxazolidin-2-yl)-N-(4-(3,3-difluoropyrrolidin-1- |
| | yl)-2-methoxy-5-(1-methyl-1H-pyrazol-4-yl)phenyl)pyrimidin-4-amine |
| 23 | 6-((R)-3-(2,3-difluorophenyl)isoxazolidin-2-yl)-N-(5-(3-fluoro-1-methyl-1H- |
| | pyrazol-4-yl)-2-methoxy-4-((R)-2-methylmorpholino)phenyl)pyrimidin-4-amine |
| 24 | 6-((R)-3-(2,3-difluorophenyl)isoxazolidin-2-yl)-N-(4-((2S,6R)-2,6- |
| | dimethylmorpholino)-5-(3-fluoro-1-methyl-1H-pyrazol-4-yl)-2- |
| | methoxyphenyl)pyrimidin-4-amine |
| 25 | 6-((R)-3-(2,3-difluorophenyl)isoxazolidin-2-yl)-N-(5-(3-fluoro-1-methyl-1H- |
| | pyrazol-4-yl)-2-methoxy-4-((S)-3-methylmorpholino)phenyl)pyrimidin-4-amine |
| 26 | (R)-6-(3-(2,3-difluorophenyl)isoxazolidin-2-yl)-N-(2-methoxy-4-(4- |
| | (methyl(oxetan-3-yl)amino)piperidin-1-yl)-5-(1-methyl-1H-pyrazol-4- |
| | yl)phenyl)pyrimidin-4-amine |
| 27 | (R)-6-(3-(2,3-difluorophenyl)isoxazolidin-2-yl)-N-(4-(2,2-dimethylmorpholino)- |
| | 2-methoxy-5-(1-methyl-1H-pyrazol-4-yl)phenyl)pyrimidin-4-amine |
| 28 | (R)-6-(3-(2,3-difluorophenyl)isoxazolidin-2-yl)-N-(2-methoxy-5-(1-methyl-1H- |
| | pyrazol-4-yl)-4-(3-oxa-9-azaspiro[5.5]undecan-9-yl)phenyl)pyrimidin-4-amine |
| 29 | (R)-7-(4-((6-(3-(2,3-difluorophenyl)isoxazolidin-2-yl)pyrimidin-4-yl)amino)-5- |
| | methoxy-2-(1-methyl-1H-pyrazol-4-yl)phenyl)-N-methyl-N-(oxetan-3-yl)-7- |
| | azaspiro[3.5]nonan-2-amine |
| 30 | (R)-6-(3-(2,3-difluorophenyl)isoxazolidin-2-yl)-N-(2-methoxy-4-(2-methoxy-7- |
| | azaspiro[3.5]nonan-7-yl)-5-(1-methyl-1H-pyrazol-4-yl)phenyl)pyrimidin-4- |
| | amine |
| 31 | (R)-7-(4-((6-(3-(2,3-difluorophenyl)isoxazolidin-2-yl)pyrimidin-4-yl)amino)-5- |
| | methoxy-2-(1-methyl-1H-pyrazol-4-yl)phenyl)-N,N-dimethyl-7- |
| | azaspiro[3.5]nonan-2-amine |
| 32 | (R)-N-(4-(1,1-difluoro-6-azaspiro[2.5]octan-6-yl)-2-methoxy-5-(1-methyl-1H- |
| | pyrazol-4-yl)phenyl)-6-(3-(2,3-difluorophenyl)isoxazolidin-2-yl)pyrimidin-4- |
| | amine |
| 33 | (R)-6-(3-(2,3-difluorophenyl)isoxazolidin-2-yl)-N-(4-(4-(3,3-difluoropyrrolidin- |
| | 1-yl)piperidin-1-yl)-2-methoxy-5-(1-methyl-1H-pyrazol-4-yl)phenyl)pyrimidin- |
| | 4-amine |
| 34 | (R)-6-(3-(2,3-difluorophenyl)isoxazolidin-2-yl)-N-(4-(4-(3- |
| | (dimethylamino)azetidin-1-yl)piperidin-1-yl)-2-methoxy-5-(1-methyl-1H- |
| | pyrazol-4-yl)phenyl)pyrimidin-4-amine |
| 35 | (R)-6-(3-(2,3-difluorophenyl)isoxazolidin-2-yl)-N-(4-(9-(1-fluoro-2- |
| | methylpropan-2-yl)-3,9-diazaspiro[5.5]undecan-3-yl)-2-methoxy-5-(thiazol-5- |
| | yl)phenyl)pyrimidin-4-amine |
| 36 | (R)-2-(4-(5-((6-(3-(2,3-difluorophenyl)isoxazolidin-2-yl)pyrimidin-4-yl)amino)- |
| | 2-(9-(1-fluoro-2-methylpropan-2-yl)-3,9-diazaspiro[5.5]undecan-3-yl)-4- |
| | methoxyphenyl)-1H-pyrazol-1-yl)ethan-1-ol |
| 37 | (R)-6-(3-(2,3-difluorophenyl)isoxazolidin-2-yl)-N-(4-(9-(1-fluoro-2- |
| | methylpropan-2-yl)-3,9-diazaspiro[5.5]undecan-3-yl)-2-methoxy-5- |
| | (pyrazolo[1,5-a]pyridin-3-yl)phenyl)pyrimidin-4-amine |
| 38 | (R)-6-(3-(2,3-difluorophenyl)isoxazolidin-2-yl)-N-(5-(3-fluoro-1-methyl-1H- |
| | pyrazol-4-yl)-4-(4-(((1-fluoro-2-methylpropan-2- |
| | yl)(methyl)amino)methyl)piperidin-1-yl)-2-methoxyphenyl)pyrimidin-4-amine |
| 39 | (R)-6-(3-(2,3-difluorophenyl)isoxazolidin-2-yl)-N-(5-(3-fluoro-1-methyl-1H- |
| | pyrazol-4-yl)-2-methoxy-4-(4-methoxy-4-methylpiperidin-1- |
| | yl)phenyl)pyrimidin-4-amine |
| 40 | (R)-6-(3-(2,3-difluorophenyl)isoxazolidin-2-yl)-N-(5-(3-fluoro-1-methyl-1H- |
| | pyrazol-4-yl)-2-methoxy-4-(4-(3-(methoxymethyl)azetidin-1-yl)piperidin-1- |
| | yl)phenyl)pyrimidin-4-amine |
| 41 | N-(4-(7,7-difluoro-3-azabicyclo[4.1.0]heptan-3-yl)-2-methoxy-5-(1-methyl-1H- |
| | pyrazol-4-yl)phenyl)-6-((R)-3-(2,3-difluorophenyl)isoxazolidin-2-yl)pyrimidin-4- |
| | amine |
| 42 | (R)-6-(3-(2,3-difluorophenyl)isoxazolidin-2-yl)-N-(2-methoxy-4-(9-methoxy-3- |
| | azaspiro[5.5]undecan-3-yl)-5-(1-methyl-1H-pyrazol-4-yl)phenyl)pyrimidin-4- |
| | amine |
| 43 | (R)-6-(3-(2,3-difluorophenyl)isoxazolidin-2-yl)-N-(2-methoxy-5-(1-methyl-1H- |
| | pyrazol-4-yl)-4-(4-oxa-7-azaspiro[2.5]octan-7-yl)phenyl)pyrimidin-4-amine |
| 44 | N-(4-(3-oxa-6-azabicyclo[3.1.1]heptan-6-yl)-2-methoxy-5-(1-methyl-1H- |
| | pyrazol-4-yl)phenyl)-6-((R)-3-(2,3-difluorophenyl)isoxazolidin-2-yl)pyrimidin-4- |
| | amine |
| 45 | (R)-6-(3-(2,3-difluorophenyl)isoxazolidin-2-yl)-N-(2-methoxy-5-(1-methyl-1H- |
| | pyrazol-4-yl)-4-(8-oxa-2-azaspiro[4.5]decan-2-yl)phenyl)pyrimidin-4-amine |
| 46 | (R)-6-(3-(2,3-difluorophenyl)isoxazolidin-2-yl)-N-(2-methoxy-5-(1-methyl-1H- |
| | pyrazol-4-yl)-4-(4-morpholinopiperidin-1-yl)phenyl)pyrimidin-4-amine |
| 47 | (R)-N-(6-(3-(2,3-difluorophenyl)isoxazolidin-2-yl)pyrimidin-4-yl)-6-isopropyl-8- |
| | methoxy-3-methyl-3,4,5,6-tetrahydrobenzo[b]pyrazolo[4,3-d]azepin-9-amine |
| 48 | (R)-N-(4-(2,2-difluoro-7-azaspiro[3.5]nonan-7-yl)-2-methoxy-5-(1-methyl-1H- |
| | pyrazol-4-yl)phenyl)-6-(3-(2,3-difluorophenyl)isoxazolidin-2-yl)pyrimidin-4- |
| | amine |
| 49 | (R)-6-(3-(2,3-difluorophenyl)isoxazolidin-2-yl)-N-(2-methoxy-5-(1-methyl-1H- |
| | pyrazol-4-yl)-4-(2-oxa-6-azaspiro[3.4]octan-6-yl)phenyl)pyrimidin-4-amine |
| 50 | (R)-6-(3-(2,3-difluorophenyl)isoxazolidin-2-yl)-N-(4-(9-(2-fluoroethyl)-3,9- |
| | diazaspiro[5.5]undecan-3-yl)-2-methoxy-5-(1-methyl-1H-pyrazol-4- |
| | yl)phenyl)pyrimidin-4-amine |
| 51 | (9-(4-((6-((R)-3-(2,3-difluorophenyl)isoxazolidin-2-yl)pyrimidin-4-yl)amino)-5- |
| | methoxy-2-(1-methyl-1H-pyrazol-4-yl)phenyl)-3,9-diazaspiro[5.5]undecan-3- |
| | yl)((1R,2R)-2-fluorocyclopropyl)methanone |
| 52 | (R)-1-(9-(4-((6-(3-(2,3-difluorophenyl)isoxazolidin-2-yl)pyrimidin-4-yl)amino)- |
| | 5-methoxy-2-(1-methyl-1H-pyrazol-4-yl)phenyl)-3,9-diazaspiro[5.5]undecan-3- |
| | yl)ethan-1-one |
| 53 | (R)-6-(3-(2,3-difluorophenyl)isoxazolidin-2-yl)-N-(2-methoxy-5-(1-methyl-1H- |
| | pyrazol-4-yl)-4-(2-oxa-6-azaspiro[3.3]heptan-6-yl)phenyl)pyrimidin-4-amine |
| 54 | (R)-6-(3-(2,3-difluorophenyl)isoxazolidin-2-yl)-N-(4-(9-(1-fluoro-2- |
| | methylpropan-2-yl)-3,9-diazaspiro[5.5]undecan-3-yl)-2-methoxy-5-(1-methyl- |
| | 1H-indol-3-yl)phenyl)pyrimidin-4-amine |
| 55 | (R)-N-(4-(4-(3,3-difluoroazetidin-1-yl)piperidin-1-yl)-2-methoxy-5-(1-methyl- |
| | 1H-pyrazol-4-yl)phenyl)-6-(3-(2,3-difluorophenyl)isoxazolidin-2-yl)pyrimidin-4- |
| | amine |
| 56 | (R)-6-(3-(2,3-difluorophenyl)isoxazolidin-2-yl)-N-(2-methoxy-5-(1-methyl-1H- |
| | pyrazol-4-yl)-4-(9-(oxetan-3-yl)-3,9-diazaspiro[5.5]undecan-3- |
| | yl)phenyl)pyrimidin-4-amine |
| 57 | N-(4-((1S,4S)-2-oxa-5-azabicyclo[2.2.1]heptan-5-yl)-2-methoxy-5-(1-methyl-1H- |
| | pyrazol-4-yl)phenyl)-6-((R)-3-(2,3-difluorophenyl)isoxazolidin-2-yl)pyrimidin-4- |
| | amine |
| 58 | N-(4-((1R,4R)-2-oxa-5-azabicyclo[2.2.1]heptan-5-yl)-2-methoxy-5-(1-methyl- |
| | 1H-pyrazol-4-yl)phenyl)-6-((R)-3-(2,3-difluorophenyl)isoxazolidin-2- |
| | yl)pyrimidin-4-amine |
| 59 | N-(4-(3-oxabicyclo[4.1.0]heptan-6-yl)-2-methoxy-5-(1-methyl-1H-pyrazol-4- |
| | yl)phenyl)-6-((R)-3-(2,3-difluorophenyl)isoxazolidin-2-yl)pyrimidin-4-amine |
| 60 | N-(4-(7,7-difluoro-3-oxabicyclo[4.1.0]heptan-6-yl)-2-methoxy-5-(1-methyl-1H- |
| | pyrazol-4-yl)phenyl)-6-((R)-3-(2,3-difluorophenyl)isoxazolidin-2-yl)pyrimidin-4- |
| | amine |
| 61 | (R)-N-(4-(4,4-difluoro-[1,4′-bipiperidin]-1′-yl)-2-methoxy-5-(1-methyl-1H- |
| | pyrazol-4-yl)phenyl)-6-(3-(2,3-difluorophenyl)isoxazolidin-2-yl)pyrimidin-4- |
| | amine |
| 62 | 6-((R)-3-(2,3-difluorophenyl)isoxazolidin-2-yl)-N-(2-methoxy-5-(1-methyl-1H- |
| | pyrazol-4-yl)-4-(1-methyloctahydro-6H-pyrrolo[2,3-c]pyridin-6- |
| | yl)phenyl)pyrimidin-4-amine |
| 63 | 6-((R)-3-(2,3-difluorophenyl)isoxazolidin-2-yl)-N-(2-methoxy-5-(1-methyl-1H- |
| | pyrazol-4-yl)-4-(4-((3-(2,2,2-trifluoroethyl)pyrrolidin-1-yl)methyl)piperidin-1- |
| | yl)phenyl)pyrimidin-4-amine |
| 64 | 6-((R)-3-(2,3-difluorophenyl)isoxazolidin-2-yl)-N-(4-((3S,4R)-3-fluoro-4- |
| | methoxy-[1,4′-bipiperidin]-1′-yl)-2-methoxy-5-(1-methyl-1H-pyrazol-4- |
| | yl)phenyl)pyrimidin-4-amine |
| 65 | 6-((R)-3-(2,3-difluorophenyl)isoxazolidin-2-yl)-N-(4-((3R,4S)-3-fluoro-4- |
| | methoxy-[1,4′-bipiperidin]-1′-yl)-2-methoxy-5-(1-methyl-1H-pyrazol-4- |
| | yl)phenyl)pyrimidin-4-amine |
| 66 | 6-((R)-3-(2,3-difluorophenyl)isoxazolidin-2-yl)-N-(4-((3S,4R)-3-fluoro-4- |
| | methoxypiperidin-1-yl)-2-methoxy-5-(1-methyl-1H-pyrazol-4- |
| | yl)phenyl)pyrimidin-4-amine |
| 67 | 6-((R)-3-(2,3-difluorophenyl)isoxazolidin-2-yl)-N-(4-((3R,4S)-3-fluoro-4- |
| | methoxypiperidin-1-yl)-2-methoxy-5-(1-methyl-1H-pyrazol-4- |
| | yl)phenyl)pyrimidin-4-amine |
| 68 | (R)-6-(3-(2,3-difluorophenyl)isoxazolidin-2-yl)-N-(3-(4-methyl-1H-imidazol-1- |
| | yl)-5-(trifluoromethyl)phenyl)pyrimidin-4-amine |
| 69 | 6-((R)-3-(2,3-difluorophenyl)isoxazolidin-2-yl)-N-(2-methoxy-4-((R)-3- |
| | (methoxymethyl)piperidin-1-yl)-5-(1-methyl-1H-pyrazol-4-yl)phenyl)pyrimidin- |
| | 4-amine |
| 70 | 6-((R)-3-(2,3-difluorophenyl)isoxazolidin-2-yl)-N-(2-methoxy-4-(4-((S)-3- |
| | methoxypyrrolidin-1-yl)piperidin-1-yl)-5-(1-methyl-1H-pyrazol-4- |
| | yl)phenyl)pyrimidin-4-amine |
| 71 | 6-((R)-3-(2,3-difluorophenyl)isoxazolidin-2-yl)-N-(4-(4-((S)-3-fluoropyrrolidin- |
| | 1-yl)piperidin-1-yl)-2-methoxy-5-(1-methyl-1H-pyrazol-4-yl)phenyl)pyrimidin- |
| | 4-amine |
| 72 | (R)-6-(3-(2,3-difluorophenyl)isoxazolidin-2-yl)-N-(5-(3-fluoro-1-methyl-1H- |
| | pyrazol-4-yl)-2-methoxy-4-(4-(3-methoxyazetidin-1-yl)piperidin-1- |
| | yl)phenyl)pyrimidin-4-amine |
| 73 | 6-((R)-3-(2,3-difluorophenyl)isoxazolidin-2-yl)-N-(2-methoxy-5-(1-methyl-1H- |
| | pyrazol-4-yl)-4-(4-(2-(trifluoromethyl)morpholino)piperidin-1- |
| | yl)phenyl)pyrimidin-4-amine |
| 74 | 6-((R)-3-(2,3-difluorophenyl)isoxazolidin-2-yl)-N-(2-methoxy-5-(1-methyl-1H- |
| | pyrazol-4-yl)-4-((3aR,6aS)-tetrahydro-1H-furo[3,4-c]pyrrol-5(3H)- |
| | yl)phenyl)pyrimidin-4-amine |
| 75 | (R)-6-(3-(2,3-difluorophenyl)isoxazolidin-2-yl)-N-(5-(furan-3-yl)-2-methoxy-4- |
| | (4-methylpiperazin-1-yl)phenyl)pyrimidin-4-amine |
| 76 | (R)-6-(3-(2,3-difluorophenyl)isoxazolidin-2-yl)-N-(2-methoxy-4-(4- |
| | methylpiperazin-1-yl)-5-(thiophen-3-yl)phenyl)pyrimidin-4-amine |
| 77 | (R)-6-(3-(2,3-difluorophenyl)isoxazolidin-2-yl)-N-(4-methoxy-6-(4- |
| | methylpiperazin-1-yl)-[1,1′-biphenyl]-3-yl)pyrimidin-4-amine |
| 78 | (R)-6-(3-(2,3-difluorophenyl)isoxazolidin-2-yl)-N-(2-methoxy-4-(4- |
| | methylpiperazin-1-yl)-5-(quinolin-3-yl)phenyl)pyrimidin-4-amine |
| 79 | (R)-6-(3-(2,3-difluorophenyl)isoxazolidin-2-yl)-N-(2-methoxy-4-(4- |
| | methylpiperazin-1-yl)-5-(pyridin-4-yl)phenyl)pyrimidin-4-amine |
| 80 | 6-((R)-3-(2,3-difluorophenyl)isoxazolidin-2-yl)-N-(4-((S)- |
| | hexahydropyrazino[2,1-c][1,4]oxazin-8(1H)-yl)-2-methoxy-5-(1-methyl-1H- |
| | pyrazol-4-yl)phenyl)pyrimidin-4-amine |
| 81 | 6-((R)-3-(2,3-difluorophenyl)isoxazolidin-2-yl)-N-(2-methoxy-4-((S)-2- |
| | (methoxymethyl)morpholino)-5-(1-methyl-1H-pyrazol-4-yl)phenyl)pyrimidin-4- |
| | amine |
| 82 | N-(4-(4-((1R,5S)-3-oxa-8-azabicyclo[3.2.1]octan-8-yl)piperidin-1-yl)-2-methoxy- |
| | 5-(1-methyl-1H-pyrazol-4-yl)phenyl)-6-((R)-3-(2,3-difluorophenyl)isoxazolidin- |
| | 2-yl)pyrimidin-4-amine |
| 83 | (R)-6-(3-(2,3-difluorophenyl)isoxazolidin-2-yl)-N-(2-methoxy-5-(1-methyl-1H- |
| | pyrazol-4-yl)-4-(4-methyl-1-oxa-4,9-diazaspiro[5.5]undecan-9- |
| | yl)phenyl)pyrimidin-4-amine |
| 84 | (R)-6-(3-(2,3-difluorophenyl)isoxazolidin-2-yl)-N-(2-methoxy-5-(1-methyl-1H- |
| | pyrazol-4-yl)-4-(9-(2,2,2-trifluoroethyl)-3,9-diazaspiro[5.5]undecan-3- |
| | yl)phenyl)pyrimidin-4-amine |
| 85 | (R)-6-(3-(2,3-difluorophenyl)isoxazolidin-2-yl)-N-(2-methoxy-5-(1-methyl-1H- |
| | pyrazol-4-yl)-4-thiomorpholinophenyl)pyrimidin-4-amine |
| 86 | (R)-6-(3-(2,3-difluorophenyl)isoxazolidin-2-yl)-N-(3-(1-methyl-1H-pyrazol-4- |
| | yl)-5-(7-(methylsulfonyl)-2,7-diazaspiro[3.5]nonan-2-yl)phenyl)pyrimidin-4- |
| | amine |
| 87 | (R)-6-(3-(2,3-difluorophenyl)isoxazolidin-2-yl)-N-(5-(1-methyl-1H-pyrazol-4- |
| | yl)-4-(4-morpholinopiperidin-1-yl)-2,3-dihydrobenzofuran-7-yl)pyrimidin-4- |
| | amine |
| 88 | (R)-6-(3-(2,3-difluorophenyl)isoxazolidin-2-yl)-N-(6-(1-methyl-1H-pyrazol-4- |
| | yl)-7-morpholinobenzo[d][1,3]dioxol-4-yl)pyrimidin-4-amine |
| 89 | 6-((R)-3-(2,3-difluorophenyl)isoxazolidin-2-yl)-N-(2-methoxy-5-(1-methyl-1H- |
| | pyrazol-4-yl)-4-((R)-octahydro-2H-pyrido[1,2-a]pyrazin-2-yl)phenyl)pyrimidin- |
| | 4-amine |
| 90 | (R)-6-(3-(2,3-difluorophenyl)isoxazolidin-2-yl)-N-(3-(1-methyl-1H-pyrazol-4- |
| | yl)-4-(4-morpholinopiperidin-1-yl)phenyl)pyrimidin-4-amine |
| 91 | (R)-6-(3-(2,3-difluorophenyl)isoxazolidin-2-yl)-N-(6-(1-methyl-1H-pyrazol-4- |
| | yl)-5-(4-morpholinopiperidin-1-yl)chroman-8-yl)pyrimidin-4-amine |
| 92 | 6-((R)-3-(2,3-difluorophenyl)isoxazolidin-2-yl)-N-(3-(1-methyl-1H-pyrazol-4- |
| | yl)-5-((2R,3S)-2-methyl-3-((methylsulfonyl)methyl)azetidin-1- |
| | yl)phenyl)pyrimidin-4-amine |
| 93 | (R)-6-(3-(2,3-difluorophenyl)isoxazolidin-2-yl)-N-(6-(1-methyl-1H-pyrazol-4- |
| | yl)-5-(4-(4-methylpiperazin-1-yl)piperidin-1-yl)chroman-8-yl)pyrimidin-4-amine |
| 94 | (R)-N-(6-((R)-3-(2,3-difluorophenyl)isoxazolidin-2-yl)pyrimidin-4-yl)-3-methyl- |
| | 10-(1-methyl-1H-pyrazol-4-yl)-1,2,3,4,4a,5-hexahydrobenzo[b]pyrazino[1,2- |
| | d][1,4]oxazin-8-amine |
| 95 | (R)-6-(3-(2,3-difluorophenyl)isoxazolidin-2-yl)-N-(4-(4-isopropylpiperazin-1-yl)- |
| | 2-methoxy-5-(1-methyl-1H-pyrazol-4-yl)phenyl)pyrimidin-4-amine |
| 96 | (R)-6-(3-(2,3-difluorophenyl)isoxazolidin-2-yl)-N-(7-(1-methyl-1H-pyrazol-4- |
| | yl)-8-morpholino-2,3-dihydrobenzo[b][1,4]dioxin-5-yl)pyrimidin-4-amine |
| 97 | (R)-6-(3-(2,3-difluorophenyl)isoxazolidin-2-yl)-N-(3-(1-methyl-1H-pyrazol-4- |
| | yl)-5-(2-(methylsulfonyl)-2,7-diazaspiro[3.5]nonan-7-yl)phenyl)pyrimidin-4- |
| | amine |
| 98 | (R)-6-(3-(2,3-difluorophenyl)isoxazolidin-2-yl)-N-(2-methoxy-5-(1-methyl-1H- |
| | pyrazol-4-yl)-4-(4-methylpiperazin-1-yl)phenyl)pyrimidin-4-amine |
| 99 | (R)-N-(5-(1-cyclopropyl-1H-pyrazol-4-yl)-2-methoxy-4-(4-methylpiperazin-1- |
| | yl)phenyl)-6-(3-(2,3-difluorophenyl)isoxazolidin-2-yl)pyrimidin-4-amine |
| 100 | (R)-6-(3-(2,3-difluorophenyl)isoxazolidin-2-yl)-N-(5-(1-isopropyl-1H-pyrazol-4- |
| | yl)-2-methoxy-4-(4-methylpiperazin-1-yl)phenyl)pyrimidin-4-amine |
| 101 | 6-((R)-3-(2,3-difluorophenyl)isoxazolidin-2-yl)-N-(2-methoxy-5-(1-methyl-1H- |
| | pyrazol-4-yl)-4-(tetrahydro-1H-furo[3,4-c]pyrrol-5(3H)-yl)phenyl)pyrimidin-4- |
| | amine |
| 102 | (R)-6-(3-(2,3-difluorophenyl)isoxazolidin-2-yl)-N-(2-methoxy-4-(4-(3- |
| | methoxyazetidin-1-yl)piperidin-1-yl)-5-(1-methyl-1H-pyrazol-4- |
| | yl)phenyl)pyrimidin-4-amine |
| 103 | (R)-N-(5-(3-fluoro-1-methyl-1H-pyrazol-4-yl)-4-(9-(1-fluoro-2-methylpropan-2- |
| | yl)-3,9-diazaspiro[5.5]undecan-3-yl)-2-methoxyphenyl)-6-(3-phenylisoxazolidin- |
| | 2-yl)pyrimidin-4-amine |
| 104 | 6-((R)-3-(2,3-difluorophenyl)isoxazolidin-2-yl)-N-(4-((2S,6R)-2,6- |
| | dimethylmorpholino)-2-ethoxy-5-(1-methyl-1H-pyrazol-4-yl)phenyl)pyrimidin- |
| | 4-amine |
| 105 | 6-((R)-3-(2,3-difluorophenyl)isoxazolidin-2-yl)-N-(4-((2S,6S)-2,6- |
| | dimethylmorpholino)-2-isopropoxy-5-(1-methyl-1H-pyrazol-4- |
| | yl)phenyl)pyrimidin-4-amine |
| 106 | 6-((R)-3-(2,3-difluorophenyl)isoxazolidin-2-yl)-N-(4-((2S,6S)-2,6- |
| | dimethylmorpholino)-2-methoxy-5-(1-methyl-1H-pyrazol-4- |
| | yl)phenyl)pyrimidin-4-amine |
| 107 | 6-((R)-3-(2,3-difluorophenyl)isoxazolidin-2-yl)-N-(4-((2S,6R)-2,6- |
| | dimethylmorpholino)-2-methoxy-5-(1-methyl-1H-pyrazol-4- |
| | yl)phenyl)pyrimidin-4-amine |
| 108 | 6-((R)-3-(2,3-difluorophenyl)isoxazolidin-2-yl)-N-(2-methoxy-5-(1-methyl-1H- |
| | pyrazol-4-yl)-4-((R)-2-methylmorpholino)phenyl)pyrimidin-4-amine |
| 109 | (R)-6-(3-(2,3-difluorophenyl)isoxazolidin-2-yl)-N-(2-methoxy-4-(4-methoxy-4- |
| | methylpiperidin-1-yl)-5-(1-methyl-1H-pyrazol-4-yl)phenyl)pyrimidin-4-amine |
| 110 | (R)-6-(3-(2,3-difluorophenyl)isoxazolidin-2-yl)-N-(4-(4-(((1-fluoro-2- |
| | methylpropan-2-yl)(methyl)amino)methyl)piperidin-1-yl)-2-methoxy-5-(1- |
| | methyl-1H-pyrazol-4-yl)phenyl)pyrimidin-4-amine |
| 111 | (R)-6-(3-(2,3-difluorophenyl)isoxazolidin-2-yl)-N-(2-methoxy-5-(1-methyl-1H- |
| | pyrazol-4-yl)-4-(7-oxa-2-azaspiro[3.5]nonan-2-yl)phenyl)pyrimidin-4-amine |
| 112 | (R)-6-(3-(2,3-difluorophenyl)isoxazolidin-2-yl)-N-(5-(1-methyl-1H-pyrazol-4- |
| | yl)-4-(4-(4-methylpiperazin-1-yl)piperidin-1-yl)-2,3-dihydrobenzofuran-7- |
| | yl)pyrimidin-4-amine |
| 113 | (R)-6-(3-(2,3-difluorophenyl)isoxazolidin-2-yl)-N-(6-(1-methyl-1H-pyrazol-4- |
| | yl)-7-(4-methylpiperazin-1-yl)benzo[d][1,3]dioxol-4-yl)pyrimidin-4-amine |

8. A pharmaceutical composition comprising the compound according to claim 1, the stereoisomer thereof, or the pharmaceutically acceptable salt thereof and a pharmaceutically acceptable additive.

9. A pharmaceutical composition for preventing or treating cancer, comprising the compound according to claim 1, the stereoisomer thereof, or the pharmaceutically acceptable salt thereof as an active ingredient.

10. The pharmaceutical composition according to claim 9, wherein the pharmaceutical composition inhibits EGFR and/or HER2.

11. The pharmaceutical composition according to claim 10, wherein the pharmaceutical composition inhibits at least one selected from the group consisting of EGFR Del19/C797S, EGFR L858R/C797S, EGFR d746-750/T790M/C797S, EGFR L858R/T790M/C797S, EGFR L861Q, EGFR G719A, EGFR S768I, EGFR L718Q, EGFR G724S, EGFR d746-750, EGFR d746-750/C797A, EGFR d746-750/C797S, EGFR D761Y, EGFR G719C, EGFR G719D, EGFR G719S, EGFR L747S, EGFR L792F, EGFR L858R, and EGFR L792F/L858R.

12. The pharmaceutical composition according to claim 9, wherein the cancer is one or more selected from the group consisting of pseudomyxoma, intrahepatic cholangiocarcinoma, hepatoblastoma, liver cancer, thyroid cancer, colon cancer, testicular cancer, myelodysplastic syndrome, glioblastoma, oral cancer, lip cancer, mycosis fungoides, acute myeloid leukemia, acute lymphoblastic leukemia, basal cell carcinoma, ovarian epithelial cancer, ovarian germ cell tumor, male breast cancer, brain cancer, pituitary adenoma, multiple myeloma, gallbladder cancer, biliary tract cancer, colorectal cancer, chronic myelogenous leukemia, chronic lymphocytic leukemia, retinoblastoma, choroidal melanoma, ampulla of Vater cancer, bladder cancer, peritoneal cancer, parathyroid cancer, adrenal cancer, sinonasal cancer, non-small cell lung cancer, tongue cancer, astrocytoma, small cell lung cancer, childhood brain cancer, childhood lymphoma, childhood leukemia, small bowel cancer, meningioma, esophageal cancer, glioma, renal pelvis cancer, renal cancer, renal cell carcinoma, heart cancer, duodenal cancer, malignant soft tissue tumor, malignant bone tumor, malignant lymphoma, malignant mesothelioma, malignant melanoma, eye cancer, vulvar cancer, ureteral cancer, urethral cancer, cancer of unknown primary site, gastric lymphoma, gastric cancer, gastric carcinoid tumor, Gastrointestinal stromal tumor, Wilms' tumor, breast cancer, sarcoma, penile cancer, pharyngeal cancer, gestational choriocarcinoma, cervical cancer, endometrial cancer, uterine sarcoma, prostate cancer, metastatic bone cancer, metastatic brain cancer, mediastinal cancer, rectal cancer, rectal carcinoid tumor, vaginal cancer, spinal cord cancer, vestibular schwannoma, pancreatic cancer, salivary gland cancer, Kaposi's sarcoma, Paget's disease, tonsil cancer, squamous cell carcinoma, lung adenocarcinoma, lung cancer, squamous cell carcinoma of lung, skin cancer, anal cancer, rhabdomyosarcoma, laryngeal cancer, pleura cancer, hematological cancer, and thymic carcinoma.

13. A pharmaceutical composition for preventing or treating an EGFR- and/or a HER2-related disease, comprising the compound according to claim 1, the stereoisomer thereof, or the pharmaceutically acceptable salt thereof as an active ingredient.

14. A method for treating or preventing EGFR- and/or HER2-related diseases comprising administering to a subject in need thereof a therapeutically effective amount of the compound according to claim 1, the stereoisomer thereof, or the pharmaceutically acceptable salt thereof.

15. A method for treating or preventing at least one disease comprising administering to a subject in need thereof a therapeutically effective amount of the compound according to claim 1, the stereoisomer thereof, or the pharmaceutically acceptable salt thereof,
wherein the at least one disease is selected from the group consisting of pseudomyxoma, intrahepatic cholangiocarcinoma, hepatoblastoma, liver cancer, thyroid cancer, colon cancer, testicular cancer, myelodysplastic syndrome, glioblastoma, oral cancer, lip cancer, mycosis fungoides, acute myeloid leukemia, acute lymphoblastic leukemia, basal cell carcinoma, ovarian epithelial cancer, ovarian germ cell tumor, male breast cancer, brain cancer, pituitary adenoma, multiple myeloma, gallbladder cancer, biliary tract cancer, colorectal cancer, chronic myelogenous leukemia, chronic lymphocytic leukemia, retinoblastoma, choroidal melanoma, ampulla of Vater cancer, bladder cancer, peritoneal cancer, parathyroid cancer, adrenal cancer, sinonasal cancer, non-small cell lung cancer, tongue cancer, astrocytoma, small cell lung cancer, childhood brain cancer, childhood lymphoma, childhood leukemia, small bowel cancer, meningioma, esophageal cancer, glioma, renal pelvis cancer, renal cancer, renal cell carcinoma, heart cancer, duodenal cancer, malignant soft tissue tumor, malignant bone tumor, malignant lymphoma, malignant mesothelioma, malignant melanoma, eye cancer, vulvar cancer, ureteral cancer, urethral cancer, cancer of unknown primary site, gastric lymphoma, gastric cancer, gastric carcinoid tumor, Gastrointestinal stromal tumor, Wilms' tumor, breast cancer, sarcoma, penile cancer, pharyngeal cancer, gestational choriocarcinoma, cervical cancer, endometrial cancer, uterine sarcoma, prostate cancer, metastatic bone cancer, metastatic brain cancer, mediastinal cancer, rectal cancer, rectal carcinoid tumor, vaginal cancer, spinal cord cancer, vestibular schwannoma, pancreatic cancer, salivary gland cancer, Kaposi's sarcoma, Paget's disease, tonsil cancer, squamous cell carcinoma, lung adenocarcinoma, lung cancer, squamous cell carcinoma of lung, skin cancer, anal cancer, rhabdomyosarcoma, laryngeal cancer, pleura cancer, hematological cancer, and thymic carcinoma.
